SEQUENCE LISTING (13 sequences)

SEQ ID NO: 1 - DNA, 4824 bp, Homo sapiens
gcggaaaagaggccgtggggggcctcccagcgctggcagacaccgtgaggctggcagccgccggcacgcacacctagtccgcagtcccgaggaacatgtccgcagccagggcgcggagcagagtcccgggcaggagaaccaagggagggcgtgtgctgtggcggcggcggcagcggcagcggagccgctagtcccctccctcctgggggagcagctgccgccgctgccgccgccgccaccaccatcagcgcgcggggcccggccagagcgagccgggcgagcggcgcgctagggggagggcgggggcggggaggggggtgggcgaagggggcgggagggcgtggggggagggtctcgctctcccgactaccagagcccgagagggagaccctggcggcggcggcggcgcctgacactcggcgcctcctgccgtgctccggggcggcatgtccgaggctggcggggccgggccgggcggctgcggggcaggagccggggcaggggccgggcccggggcgctgcccccgcagcctgcggcgcttccgcccgcgcccccgcagggctccccctgcgccgctgccgccgggggctcgggcgcctgcggtccggcgacggcagtggctgcagcgggcacggccgaaggaccgggaggcggtggctcggcccgaatcgccgtgaagaaagcgcaactacgctccgctccgcgggccaagaaactggagaaactcggagtgtactccgcctgcaaggccgaggagtcttgtaaatgtaatggctggaaaaaccctaacccctcacccactccccccagagccgacctgcagcaaataattgtcagtctaacagaatcctgtcggagttgtagccatgccctagctgctcatgtttcccacctggagaatgtgtcagaggaagaaatgaacagactcctgggaatagtattggatgtggaatatctctttacctgtgtccacaaggaagaagatgcagataccaaacaagtttatttctatctatttaagctcttgagaaagtctattttacaaagaggaaaacctgtggttgaaggctctttggaaaagaaacccccatttgaaaaacctagcattgaacagggtgtgaataactttgtgcagtacaaatttagtcacctgccagcaaaagaaaggcaaacaatagttgagttggcaaaaatgttcctaaaccgcatcaactattggcatctggaggcaccatctcaacgaagactgcgatctcccaatgatgatatttctggatacaaagagaactacacaaggtggctgtgttactgcaacgtgccacagttctgcgacagtctacctcggtacgaaaccacacaggtgtttgggagaacattgcttcgctcggtcttcactgttatgaggcgacaactcctggaacaagcaagacaggaaaaagataaactgcctcttgaaaaacgaactctaatcctcactcatttcccaaaatttctgtccatgctagaagaagaagtatatagtcaaaactctcccatctgggatcaggattttctctcagcctcttccagaaccagccagctaggcatccaaacagttatcaatccacctcctgtggctgggacaatttcatacaattcaacctcatcttcccttgagcagccaaacgcagggagcagcagtcctgcctgcaaagcctcttctggacttgaggcaaacccaggagaaaagaggaaaatgactgattctcatgttctggaggaggccaagaaaccccgagttatgggggatattccgatggaattaatcaacgaggttatgtctaccatcacggaccctgcagcaatgcttggaccagagaccaattttctgtcagcacactcggccagggatgaggcggcaaggttggaagagcgcaggggtgtaattgaatttcacgtggttggcaattccctcaaccagaaaccaaacaagaagatcctgatgtggctggttggcctacagaacgttttctcccaccagctgccccgaatgccaaaagaatacatcacacggctcgtctttgacccgaaacacaaaacccttgctttaattaaagatggccgtgttattggtggtatctgtttccgtatgttcccatctcaaggattcacagagattgtcttctgtgctgtaacctcaaatgagcaagtcaagggctatggaacacacctgatgaatcatttgaaagaatatcacataaagcatgacatcctgaacttcctcacatatgcagatgaatatgcaattggatactttaagaaacagggtttctccaaagaaattaaaatacctaaaaccaaatatgttggctatatcaaggattatgaaggagccactttaatgggatgtgagctaaatccacggatcccgtacacagaattttctgtcatcattaaaaagcagaaggagataattaaaaaactgattgaaagaaaacaggcacaaattcgaaaagtttaccctggactttcatgttttaaagatggagttcgacagattcctatagaaagcattcctggaattagagagacaggctggaaaccgagtggaaaagagaaaagtaaagagcccagagaccctgaccagctttacagcacgctcaagagcatcctccagcaggtgaagagccatcaaagcgcttggcccttcatggaacctgtgaagagaacagaagctccaggatattatgaagttataaggttccccatggatctgaaaaccatgagtgaacgcctcaagaataggtactacgtgtctaagaaattattcatggcagacttacagcgagtctttaccaattgcaaagagtacaacccccctgagagtgaatactacaaatgtgccaatatcctggagaaattcttcttcagtaaaattaaggaagctggattaattgacaagtgattttttttcccctctgcttcttagaaactcaccaagcagtgtgcctaaagcaaggtggtttagttttttacaaagaattggacatgatgtattgaagagacttgtaaatgtaataattagcacttttgaaaaaacaaaaaacctccttttagcttttcagatatgtatttaaattgaagtcataggacatttttattttatggaatagattttaatctatttactactattaaggtaaattttctatggcatgtccattagctatttcatgatagatgattaggggtttcctcaaaacctgtgtgtgaggaaattgcacacagtagcaaaatttggggaaatccataacattttcagaccatgaatgaatgtttccatttttttctaatggaatgtgagagtttacttttattttattctgaaggactttaaggaagggatacatgattttaaaaaagcctgtaagaggtgaaatatgtgatgtttgaagtctctttatagactttttatatatattttttaaaacactcatctagatgaggtgctttgagcagttctgaaaaatgcagttccaggaaagcaactgctttggttcctaaggaagaaattctaaataatgcaaacttttaaaataagcatctaggtttttgataattctgtctacttacaacaaacttgttagtacataaccactattttaataattattttctctacacaaatgtgtaatatcatatttgactttgcttatgcaggccataagttccaaaagataatttccctgcccacaaaggcataaacttgaaaacacatgagattgaatcaacatgctttaataggaaaagatgtatggtctatatatgtatcaatctggtgaatcctcgttctaataaaggttctttttcttttctatgatacacacagccacgctgataatatgcaaatgaacattttcctttatgtctctccagataatgtttattgtctgaggtaaattaaattcccaccagggtttgctgtcagtattttaacacccacattagtatatgcgtccagggtcataaccccctaaaatccatcatgcaaccttattaatctgtcttgggattccagtttagtgcttggatttatttcctgattacactacatagaaaagtgagacatctgccattcccaactctgggaaaaccaactaatatacaaccatataaatgaaggccatcttgatggtctcaacactaatttttatgatgcaaatttatacactgatttttgtaaaggacaaagttttaaaagcgtatttaacttgatgttttctatcagcataaataaaatggtcatgaatagtcattaaaaacagttgccagtgataatctgcatgaaggaaaaagaaccctgcaaatggctattgagttggaagtattgtttttgatatgtaagagatattcagaatgctcacactgaaaatgcctcaactttttaaagtgtaagaaaccaccatgagtggtgtctagatttctaatgaagaatcatgatacagtttggattaagtatcttggactggttttaaacagtgctttgtaccggatctgctgaagcatctgtccagctggtatcctgtgaaagtttgttattttctgagtagacattcttatagagtattgtctttaaaatcagattgtctcttctatattgaaagcatttttatgttttctaatttaaaaattaatattttcttatagatattgtgcaataaagctgaagtagaatgtgtggtttttgcaaatgctttaacagctgataaaaattttacatttgtaaaattaatatattgtactggtacaaaatagttttaaattatattttaaaaagcttccaa

SEQ ID NO: 2 - DNA, 8298 bp, Homo sapiens
ccgttttcgaagccctccacgctgcggccgctatcccctccggaccatggccgacgacgacgtgctgttcgaggatgtgtacgagctgtgcgaggtgatcggaaagggtcccttcagtgttgtacgacgatgtatcaacagagaaactgggcaacaatttgctgtaaaaattgttgatgtagccaagttcacatcaagtccagggttaagtacagaagatctaaagcgggaagccagtatctgtcatatgctgaaacatccacacattgtagagttattggagacatatagctcagatggaatgctttacatggttttcgaatttatggatggagcagatctgtgttttgaaatcgtaaagcgagctgacgctggttttgtgtacagtgaagctgtagccagccattatatgagacagatactggaagctctacgctactgccatgataataacataattcacagggatgtgaagccccactgtgttctccttgcctcaaaagaaaactcggcacctgttaaacttggaggctttggggtagctattcaattaggggagtctggacttgtagctggaggacgtgttggaacacctcattttatggcaccagaagtggtcaaaagagagccttacggaaagcctgtagacgtctgggggtgcggtgtgatcctttttatcctgctcagtggttgtttgcctttttacggaaccaaggaaagattgtttgaaggcattattaaaggaaaatataagatgaatccaaggcagtggagccatatctctgaaagtgccaaagacctagtacgtcgcatgctgatgctggatccagctgaaaggatcactgtttatgaagcactgaatcacccatggcttaaggagcgggatcgttacgcctacaagattcatcttccagaaacagtagagcagctgaggaaattcaatgcaaggaggaaactaaagggtgcagtactagccgctgtgtcaagtcacaaattcaactcattctatggggatccccctgaagagttaccagatttctccgaagaccctacctcctcaggacttctagcagcagaaagagcagtctcacaggtgctggacagcctggaagagattcatgcgcttacagactgcagtgaaaaggacctagattttctacacagtgttttccaggatcagcatcttcacacactactagatctgtatgacaaaattaacacaaagtcttcaccacaaatcaggaatcctccaagcgatgcagtacagagagccaaagaggtattggaagaaatttcatgttaccctgagaataacgacgcaaaggaactaaagcgtattttaacacaacctcatttcatggccttacttcagactcacgacgtagtggcacatgaagtttacagtgatgaagcattgagggtcacacctcctcccacctctccctatttaaacggcgattctccagaaagtgctaacggagacatggatatggagaatgtgaccagagttcggctggtacagtttcaaaagaacacagatgaaccaatgggaatcactttaaaaatgaatgaactaaatcattgtattgttgcaagaattatgcatgggggcatgattcacaggcaaggtacacttcatgttggtgatgaaattcgagaaatcaatggcatcagtgtggctaaccaaacagtggaacaactgcaaaaaatgcttagggaaatgcgggggagtattaccttcaagattgtgccaagttaccgcactcagtcttcgtcctgtgagagagattccccttccacttccagacagtccccagctaatggtcatagcagcactaacaattctgtttcggacttgccatcaactacccaaccaaaaggacgacagatctatgtaagagcacaatttgaatatgatccagccaaggatgacctcatcccctgtaaagaagctggcattcgattcagagttggtgacatcatccagattattagtaaggatgatcataattggtggcagggtaaactggaaaactccaaaaatggaactgcaggtctcattccttctcctgaacttcaggaatggcgagtagcttgcattgccatggagaagaccaaacaggagcagcaggccagctgtacttggtttggcaagaaaaagaagcagtacaaagataaatatttggcaaagcacaatgcagatcttgtcacatatgaagaagtagtaaaactgccagcattcaagaggaaaacactagtcttattaggcgcacatggtgttgggagaagacacataaaaaacactctcatcacaaagcacccagaccggtttgcgtaccctattccacatacaaccagacctccaaagaaagacgaagaaaatggaaagaattattactttgtatctcatgaccaaatgatgcaagacatctctaataacgagtacttggagtacggcagccacgaggatgcgatgtatgggacaaaactggagaccatccggaagatccacgagcaggggctgattgcaatactggacgtggagcctcaggcactgaaggtcctgagaactgcagagtttgctccttttgttgttttcattgctgcaccaactattactccaggtttaaatgaggatgaatctcttcagcgtctgcagaaggagtctgacatcttacagagaacatatgcacactacttcgatctcacaattatcaacaatgaaattgatgagacaatcagacatctggaggaagctgttgagctcgtgtgcacagccccacagtgggtccctgtctcctgggtctattaggcctctccccagatatctgagcataactgggagcacctcatttgtggaaaagcctctttgttatcggccttgtgtcagcaggtcatggtccctagagactacctagttgtagtgtgacctacatttataattattgtcatgtccgaatagataggaggagaaaaacaattacacactaatttaaagagacagtatcttttttaatcagttctcctaaactttaataaaatgtatctttaaatgtatgtattattcaatcctttggaatgttatatttttggaaatcatagctttttatttccaaggcccctaaaaactgcacaaaatagatgctgctttctataatctattttaataataataaacaatgattctgttaccttgactgggggtggaacactacattctttttagagtctgattttatggattggaatattgggatatctttctttcctttatttatttgaaataattagtgtagtgattacaaaacagtcataaatttttaaaggccttttttctctctttttttttttttagaatagtatttttttttaagtcctttatgtaacatccagataatgtgatactgtctctttgaagcaccctgtaaaccttttagagatttgaagttgggtcttgactcttaatgcatgtggacagtcgcgagcgtttatgctgtcggtgtgtctgtgttggacaaaacaatctgtaatctacagcaaagtacattctacattccgttcatggggcacacccaggggaataagaataaaatgctattatgactaagttgtaaacctatgcacatcccttgcattttgggcaactttataaaaaaaaagaaactgatttttattaataataatcatgtagtgaaatgtgtttgtaattttgtctcaatttaatttgttgtaaggtggggtggggggaattgctggtttcaccatttcagatctgtgttgtctaagagtattaacgttttaattaagcaaagaaatgatttttaatctgtatgtaattgttttaaagcacccattttaagagaaaatactgtgcaatgaagaaaccagtttaggcatttgctataaactgaaatattccaaaagaatcatctataacagccctgtaaattcctttaaaatgataactaacaggacagtttgaccaattttttttaaatatacttccttttatgtgttcaataattaaatgcctttgggtccttcatttcattatagatttgttcaggcttccaagctgataatctttacaattgtataatttgttagatgctcattgaacctttgaaggcccaggagtgagtatcagttggcagggtgacaaagtagcctgccagagcacagctggaccacgtctgactcgcacagagccatgcgcccaagggccacgggtgtactggaggccacctgtggtctctccttcctcttgctgtatcccttggcacaggggaatcatctgtccttgttgctttagagacattaaccatctcataactcttccatgaagtcacattcttccaatacaggagattcgggctggggttctcagactctttgccagaggtcagtagaatgtttttcattagcatgtgagggtgtgaagtgcttatattaacctgtgatccttgaaaacataccatcattttttgcttcttgtaggttagatggccttgtcactttgtgccttggcaggatgtgcagggtgtgttctgagctgaacagctcctcttaaaggaccaaacagagaaggcattagtatacttcttttacattgtcaacaccatacccgcctccgccccccaccgcctattattaaatggtggcgatttttttttttctgatgttgttctttgccagtgaccagatgagtctgcaacttactaacaaggtattgagtgaactacttctgtatgggccccacagtggggtgaccaagccaccaaaccagagataagctcaacagtggtctcacactagatagagagagaaaccttcaaagagttgggtagggctgtgttagaactgattcgaatctgatagatgccaaggccagacagtcgttcctggtatggtactgaccacaatttcgggctcttagtgtgaaggtgcgcactctacctcttttctggtgaagcacaaagacaactacaactaacacatgggaaacagaaacttccctccctctagtggaaacccccacgcccccacttgccctttgagcccacatctccagagatgctgatgatgcctgtcttaagccatctgccatatttataaaggtggattaagcagaggaggagaaaagcaatgccaacttaaatgtctgtggccccacaattcacaagcactgtagcactcatcctcagtggctttatacgtcctctgagaagcacacaggcaggcatcattcttgccatcattccgatggtgaaatccctgcatgcagaactcattgacttctcaaagatgttacctgttccattccactaatacatgtcttctctaaagcagtgtctttatgacctagctgcatatacttacatttctgaattcagagtaatttcagtacattgctgctgctgctgctgctgctgctgctgctgctgctgctgctgctgctgctactgctacattacaattgtccactaagcaatcagagaaaattgctaaaagcttctttactgcttccattccatcagcagaacctttttttttctttctttcttttaaagaccagaatgaatgtttagagtactaaatcttattagtggtcaattgaagtattggagagccacattttgaaggttctctcccccacccaactccattccccacagcaagaaagggagtgtttttatatgttgaattctttgttaaaaagatgaaatgtcctgattttccagtcacaagacagtaattactttgcacatacagcatatcccattttaatgctaaaaagggaaatgtcactcaatttaattcatctggaagtttcccagtgtcaagataacattgtagcctttttaaatgcaattaaatgtatctcttgtcaggtgagatgatttaaaatctttgctcagagaggaaaacaaatggttgacctacttcttgctatttgcattcagaaggcatcgtcccagcccatcctattggtggcagaatatctaaagatgtcgcaaagtcccccactgcagtagaaatgcaggaggtgttcctgttccccgtccaattccttcttaggctgctagagacagccagtgggccaagccttcccctgtgctccaggcaggcctgggctgcagctactgttacctttttacagcctttgatctgatcgctgcagagttcttttgtttttaaatcacttgtatttgtctcttcattaaggcttcaagtttatttcttgaaaaagtttagtcaattgaaccaccctccaggaagattcccagcgtggaaactgcccctgaaaaacaaggcagcccaaagccagctctcttttgtcccccactgatgtcatgtcctttttttttttcatttcaggccagtttcttaacttggggcaaagagaatgtgttgctaaagctaccaagaaaactgcttgattaacaaattcttaatagaaatgcctcccacactgtctctttgaaaagagaaggcaccttggaatcggcattccaaaaccaagctccttgtcatcctcatccctcctccttcttgcttcctattatcaaactcgtgtgtgtttcttgttttctcagaactgaacacagcctcctaattccatcctcttgactagggcttacaatactgagagcgattacaggctgtaataagttaaaggtccctgagcgtggctttcttgttctgcgcctctaattaattcttctattaaaagatgctttccttctcactgcagcttacagaaaggctacaagctttaatcacagatcatttcatttgccagagcctttccttttctaactcctttagaaataagatttccctctctgtttggaatctctggatacaggactctcctccaagctcatggcttgattggctttgcctgtgcacacacacatcagtttgccttaaacttggagctatggggagtcccagattttccagcttagtgggtgaagtttagctgtcacctacatccaggaagtgggggaaaagggaaaaaaagaggatattcaccaccatgttcccatccggtctttcccctgccttgctttgtgcagcaggtgggcagacagagcccacagaaacagaagtagaaagagggccgcaccatctctggggccagcactcctaagtcttagctctttgtgggaatagaaaatcctgagctcccaccacctctcgtctgaccctccatcaactccctcgaacctgtgcaaatggactgagcaaaagaaaatggaaagagatgtttgtcaccacacaggagacctcctttctggccccaagcacaactccatctctgcgtatgctcagaaactcagaatttagctgctacttatgtgttagctccattctctctggtgtgtgactgagagaacacgccagtgttcttactgacagaggcttaggggtgaccctctctggacccagaggaagtacaacctctgtagcgagaattgtccttgggaacagaaacaggcattcctccacagtgagtggctccccctattttttttttatttggaagagaccaagaattgggggaattgatttccagtcttagctgtttcccggaatgtcatctgtgcaggcaggtaggcggctgccgggtgcagcaggcagcgtgactgatgtgaaggccgcccaatcctcacgtgctaagacccgagtatcgtgtttaattgcagtgtaggcacgtcgccattcaaactcagttctggggtggttttgcataaagttttgccaacgtgttcttttttctgtatgtattattgcctttttataaaaggtgttgaagtattgtctcagtgataaaaagagagagatgttaatggttgttgtatatttcaccgtatccaattgtaagtatttgcagggtacagcagagccttagcttgcaagagcgtttgtgcaggaggtgtccctcaacacccaggagctgaagggattggaaaggtttggattctttgtaaatgtcctgcttttctttctctctgtgtgtatctatttacatgccttagcacacgccctcccatcctgatccctttgccctgtcgccggaacaaccagaatgctggtgcgctgccggcacagtacaagttgatgaaaccctaatcagttatcttgtgtgacaggggaagaagagaaactccatagtattccttgtagaatatacatacctgtaggatgctgtgtaatgggaaaccatagaattgggcttttgtcatttcaaagttggagaaatgtatgaataaaatgtataaaacatgaaaa

SEQ ID NO: 3 - DNA, 5341 bp, Homo sapiens
agctagagccggttttgttcgccagcagatggcctgattcgacctctccaaaaatagacattttaactctctgaactcctgtttaaggaaggatgtaattcgctttgctctctcctcattttcaaggttcaaagggaagctgcgaggattccaaggtcccaccgccctcacagccaatgaggggcctgggagggaggagcttggtgcagcttgagcttctgagagaatcattcccggtagatgcagcggagtctgagctctgctgcatctgtcacagcagaacaaaattaaaaacacaacagtggaagagaaacgctgcagactatgggacgctgtaggactttctaaaacatttgctggggatttctgtgaagcatgatcttttaaaacgaattcttttggaagccggtttgggtaactgggaaaatgacacatatgctaaatgcagcagctgatcgagtgaaatggaccagatcgagtgctgctaagagggctgcctgcctggtggctgcggcatatgctctgaaaaccctctatcccatcattggcaagcgtttaaagcaatctggccacgggaagaaaaaagcagcagcttaccctgctgcagagaacacagaaatactgcattgcaccgagaccatttgtgaaaaaccttcgcctggagtgaatgcagatttcttcaaacagctactagaacttcggaaaattttgtttccaaaacttgtgaccactgaaacagggtggctctgcctgcactcagtggctctaatctcaagaacctttctttctatctatgtggctggtctggatggaaaaatcgtgaaaagcattgtggaaaagaagcctcggactttcatcatcaaattaatcaagtggcttatgattgccatccctgctaccttcgtcaacagtgcaataaggtacctggaatgcaaattggctttggccttcagaactcgcctagtagaccacgcctatgaaacctattttacaaatcagacttattataaagtgatcaatatggatgggaggctggcaaaccctgaccaatctcttacggaggatattatgatgttctcccaatctgtggctcacttgtattccaatctgaccaaacctattttagatgtaatgctgacctcctatacactcattcaaactgctacatccagaggagcaagcccaattgggcccaccctactagcaggacttgtggtgtatgccactgctaaagtgttaaaagcctgttctcccaaatttggcaaactggtggcagaggaagcacatagaaaaggctatttgcggtatgtgcactcgagaattatagccaatgtagaagaaattgccttttacagaggacataaggtagaaatgaaacaacttcagaaaagttacaaagctttagcagatcagatgaacctcattttatccaaacgtttgtggtacatcatgatagaacagttcctgatgaagtatgtttggagcagcagtggactaattatggtggctatacctattatcactgcaactggctttgcagatggtgaggatggccaaaagcaagttatggttagtgaacggacagaagcctttaccactgctcgaaatttactggcctctggagctgatgctattgaaaggattatgtcttcatacaaagaggtcactgaattagcaggctacactgctcgagtgtacaatatgttttgggtctttgatgaagtaaaaagaggcatttataagagaactgctgtcattcaagaatctgaaagccatagcaagaatggagctaaggtagaattacctctcagtgacacattggcaattaaaggaaaagttattgatgtggatcacggaattatttgtgaaaatgttcccataattacaccagcaggagaagtggtggcttccaggctaaacttcaaagtagaagaaggaatgcatcttttgataactggtcccaatggttgtgggaaaagttctctcttcagaattctaagtgggctctggcctgtgtatgaaggagtcctctataaaccacctcctcaacatatgttttatattccacaaaggccatatatgtctcttggaagtcttcgggatcaagtcatttaccctgattcagtggatgatatgcatgataaaggttatacagaccaagatctggaacgtatcctacacaatgtccatctctatcacatagttcaaagagaaggaggatgggatgctgttatggactggaaagatgtcctgtcaggaggggaaaagcaaagaatgggcatggctcgtatgttttatcataaaccaaaatatgccttgctggatgaatgtaccagtgctgtcagcattgatgtcgaaggaaagatatttcaggctgcaaaaggggctggaatttccttactgtctataacacacagaccttctctttggaaataccacacacatttattacagtttgatggtgaaggaggttggcgctttgaacaattggatactgctatccgtttgacattgagtgaagaaaaacaaaagctagaatctcagctagctggaattcccaaaatgcagcagagactcaatgaactatgtaaaattttgggagaagactcagtgctgaaaacaattaaaaatgaagatgagacatcttaatttgttttgacatattttaaaagttaattattagataaaggctcaaagacattctgttatactgcatgaagtatgttaagctaagcacagagaaaaaaaggcagcaagacatgttttataagattttagcattaaggaagtatatgatctgacttttcagaagaaaataaacaaatgcattatgtaaggtcagtcattatgacttatactaattcctagtgaaggcctaatgcacttgtaaaacaggattttctaggtgaattcatgatgaataccagatttactatgtatatgtggtgtgtctgaagttcttaacaaacatgggcaatattctggaaatgaaacaagttaaactgagcagcatttgggttgataccaagtgcataagattcaaactttgagtgacatttagtccatttatggttgatattaggtttaatagctagaattcaaattgattattgctagtggccaactaaacctgtacaaaatagctgacagttttataaataatttcaatataaaaattgttttaatggcatttgttgaaagaaaaaagcatggctaaaatgtataaaatgccatatttttaaattttggactttaagcatcttaatgagggcatataacaaattaattttagtacaaatcttaaatatttttaataaatcctttcattttaaaaagagaattgccaatacagaaaaggagtatccaaacaatgtctcaacctgataatttccttagcagaattacctattgcaacttctgttcagaaatacacagcttgtttttttgcccaaggatgagtctacattttaagaactgcaatggtataaaggaacttaaggattctgagaatcatagtaataacatacattggaatagtactttataatttacaatccccatttacatcatttcaccttaatgttgatgacaatgttttgaaacaaatactatttttcctactttgcttttgagaaaattgacactcagacttgccctaatcatgcactttacttaaggaaagatcgagaaatcaaatgaagttctcctgactctctggtttagtgctcttttgttattatcctttaaatcaaactgggctataatagcaataaaagttagacgaagtgtagaaaataaaataaatttcataatgttagtcttattgttatttggttattgtttacaagaaatgaaaattaagtacaaagtgcaaagatcattgttctgcggcttaaaatgaaatgagaaagttagtaaatcattcagcaactatgcagtcttactcaatattaatgtacctctaaggcattagcaatgagaagggcaaataattatggttttggtaaagttaatatttattgaacataatgagaaataatctatatggactagtcatatgaaatagattttattctatttgtgtgcactggagctggaaagccaagactgaaactttatctcttgctgcctgtgataccacattgacataccagaagaatattggaatgatcagttgttagtggcctaggattttatttgcctgttgagacacagccataatagatgttaagtctgtgattcttagacttttcaggtgatagagtacctggaagtcattcataatgatcgttatagaacatcataacatttctacattttcaattctgccatgatagctagtgtatttttaattttcaataaatgtaaccaacttacatgaaggaatgcaatataaaattataatcagtacatttgtccaacgttttcattagtattatcacctacttattcatcagttactaatatttcctgttagaaaagtgaggcaggaattaaagatttttttttaagaagcatggaaccaaaacttaaacttgctaaaaattattcattaatcttttctacataagtgattatccagtttatactgctacaagggcaaaagtctgaggcaagtgttttaagttaacttttaaaggtacaagaagaaaatgaaaaatcctcatgtgaaagatgtgtataattttcatgccttaattttcataatataaaaataaaacataattttaaaacaacctgataattttgttttgaaacattgaaaagaatgccaaaacaaatattagatttgtaattagtcagtatttaatatattttacccatgagctaaagcaaaaaagactcattatttatgatgcacaggattcaaagatgcctaaaattctgtattaaaaactatgtacactgtaatttaaatgttaaagttctaatatgcacatgttttccatgagctttttgtgcctatttgttttctgaatttatttttcaggaccaacatccttaacttggaagggaaaacatctctgatttgaacttgtatttatgtaccattcaattgtttccaaatggtccaatgggtataccatattttactactcttatattttacggaaaacatgaaaaagactgagaaactagccatttttgaatacagcaagggatagatgaggaaaggaaaaaagaaagaatatttcaagtttcaaactacttttaccatgttatatctatttgcccaaacgtttgacatatttttggtcaaacaaaaagctataagtttatggagcttgagtttaaaatgtgcattctttaggttaactcttaaacttgaaaaataagttgg

SEQ ID NO: 4 - DNA, 5034 bp, Homo sapiens
gttggaaacggcactgctgagtgaggttgaggggtgtctcggtatgtgcgccttggatctggtgtaggcgaggtcacgcctctcttcagacagcccgagccttcccggcctggcgcgtttagttcggaactgcgggacgcgccggtgggctagggcaaggtgtgtgccctcttcctgattctggagaaaaatgccggtccggaagcaagatacccagagagcattgcaccttttggaggaatatcgttcaaaactaagccaaactgaagacagacagctcagaagttccatagaacgggttattaacatatttcagagcaacctctttcaggctttaatagatattcaagaattttatgaagtgaccttactggataatccaaaatgtatagatcgttcaaagccgtctgaaccaattcaacctgtgaatacttgggagatttccagccttccaagctctactgtgacttcagagacactgccaagcagccttagccctagtgtagagaaatacaggtatcaggatgaagatacacctcctcaagagcatatttccccacaaatcacaaatgaagtgataggtccagaattggttcatgtctcagagaagaacttatcagagattgagaatgtccatggatttgtttctcattctcatatttcaccaataaagccaacagaagctgttcttccctctcctcccactgtccctgtgatccctgtcctgccagtccctgctgagaatactgtcatcctacccaccataccacaggcaaatcctcccccagtactggtcaacacagatagcttggaaacaccaacttacgttaatggcacagatgcagattatgaatatgaagaaatcacacttgaaaggggaaattcagggcttggtttcagcattgcaggaggtacggacaacccacacattggagatgactcaagtattttcattaccaaaattatcacagggggagcagccgcccaagatggaagattgcgggtcaatgactgtatattacgagtaaatgaagtagatgttcgtgatgtaacacatagcaaagcagttgaagcgttgaaagaagcagggtctattgtacgcttgtatgtaaaaagaaggaaaccagtgtcagaaaaaataatggaaataaagctcattaaaggtcctaaaggtcttgggtttagcattgctggaggtgttggaaatcagcatattcctggggataatagcatctatgtaaccaaaataattgaaggaggtgcagcacataaggatggcaaacttcagattggagataaacttttagcagtgaataacgtatgtttagaagaagttactcatgaagaagcagtaactgccttaaagaacacatctgattttgtttatttgaaagtggcaaaacccacaagtatgtatatgaatgatggctatgcaccacctgatatcaccaactcttcttctcagcctgttgataaccatgttagcccatcttccttcttgggccagacaccagcatctccagccagatactccccagtttctaaagcagtacttggagatgatgaaattacaagggaacctagaaaagttgttcttcatcgtggctcaacgggccttggtttcaacattgtaggaggagaagatggagaaggaatatttatttcctttatcttagccggaggacctgctgatctaagtggagagctcagaaaaggagatcgtattatatcggtaaacagtgttgacctcagagctgctagtcatgagcaggcagcagctgcattgaaaaatgctggccaggctgtcacaattgttgcacaatatcgacctgaagaatacagtcgttttgaagctaaaatacatgatttacgggagcagatgatgaatagtagtattagttcagggtcaggttctcttcgaactagccagaagcgatccctctatgtcagagccctttttgattatgacaagactaaagacagtgggcttcccagtcagggactgaacttcaaatttggagatatcctccatgttattaatgcttctgatgatgaatggtggcaagccaggcaggttacaccagatggtgagagcgatgaggtcggagtgattcccagtaaacgcagagttgagaagaaagaacgagcccgattaaaaacagtgaaattcaattctaaaacgagagataaagggcagtcattcaatgacaagcgtaaaaagaacctcttttcccgaaaattccccttctacaagaacaaggaccagagtgagcaggaaacaagtgatgctgaccagcatgtaacttctaatgccagcgatagtgaaagtagttaccgtggtcaagaagaatacgtcttatcttatgaaccagtgaatcaacaagaagttaattatactcgaccagtgatcatattgggacctatgaaagacaggataaatgatgacttgatctcagaatttcctgacaaatttggatcctgtgttcctcatacaactagaccaaaacgagattatgaggtagatggaagagattatcattttgtgacttcaagagagcagatggaaaaagatatccaggaacataaattcattgaagctggccagtataacaatcatctatatggaacaagtgttcagtctgtacgagaagtagcagaaaagggcaaacactgtatccttgatgtgtctggaaatgccataaagagattacagattgcacagctttaccctatctccatttttattaaacccaaatccatggaaaatatcatggaaatgaataagcgtctaacagaagaacaagccagaaaaacatttgagagagccatgaaactggaacaggagtttactgaacatttcacagctattgtacagggggatacgctggaagacatttacaaccaagtgaaacagatcatagaagaacaatctggttcttacatctgggttccggcaaaagaaaagctatgaaaactcatgtttctctgtttctcttttccacaattccattttctttggcatctctttgccctttcctctggagtctttcttgagtactgatttcatgttgaattgtatcccacacatcatggtctgcagcttctttttcacatgtagtgtctccttcaagttacatcgtgtgtattatttaatgtcactattggttagtggccatttttcagaactgaagatggaatggcctgaccagctattaagaacgtggggagacgcagaaattgtggtaaaattccttaatgtttaagggaaagtaactttaagagatttttggaaaagctttatatacattcttttcaaatttcagtacaaatgaaaaagtggttttaatcagtgatttagtagactttgagcaactgtgcacgcttcagtttaatagcatggtttggccagtgtattactctcaagtccttttctcaatcaacttctatcatcaaagcaattgtttcattatagataaataaggacattttttaatttaaaaattcatgtctgagttgactttcataagggatttcattttttcctcaacattcttaaagccttttagtatttgacggttcttttttcccaggacatttgctaggaataacaacgtttcaatgtttttaatctacttgagcaacactatcgtgtcttacaaaagttgttcatatgtaaatgatcatcacatttcgtgaattgaggccatgttcaggtgctaaggaagttcgccttttacacagaagattgagaaaatttcctagatataaatacagataaatcagacgttacagtggtgacgtagtaaccatcatggcaatggaaaggagtccaattcatagcctaaaacttcaaatgtattcttaggagtcagattttactgaatattttacccacaatagctgcctattttgttataataaaatatatataaatatatatataaaactttctttaaactgtaactatgggaattattttctttacatagttgcgcacacacacaaacatatatatatatttaaaatatattttgtttcttttgccacctactcctaactttttgtttggtttttaaattaaatattaattgaatagacttatcttccttaatcatgtgaactgaaaatgggggcatggtggtcatgaggaggaaacatttagggaaattagattataagtaaaagtgtgggcatattctcctcttttctacaaaggttttcaaatggttcctgaggttttttgttgttgtccgtgttgttactgttgttcttgtcatcaggtttgattttggtccttgccctttccttctagttctccttttattaataggaaggcaggcaaaagccccatttatgtgtgtgttttcccctcagacagctttcatccactgctctgcactagaattgcacaaatcttcatggtgagcaattttaagaaatgttagtgaaaggtagaaattatttcacaaatcagtttctctggtccttcatattaataataatatttggcttcccattgctctttggagttgtttattaaatatgtgtttttgacaacctcctcattagtttcttaaatgagtactggtttgtaaagaattatcaacattatccattccatttatgaagaagaggagaacagctaataaactgtattgtaaaatccatatgttaagtgtgtcttgaattttgaaagaaaaaatatattttgcaagctaacattttcttgaaacaatttgaggcatcatgtaacttataaccgaatccaagagccgttaggcagcagagtgtgttaccacattgaaatacacagtgctgctgttagactaaatgtcgtaggttgttaaccacatagaaacacactagtatgaagaaaactgttgtaaaatctcaagagcttcagaaactgccttacaagaccgcagcataagctattttgaagtatttaccaaatagtcacatgttgtaaaatatcaagtggttataaaaggatgccatttatatattaaaatttacataacattgttttctgga

SEQ ID NO: 5 - DNA, 3347 bp, Homo sapiens
gagaggccggcgtctctcccccagtttgccgttcacccggagcgctcgggacttgccgatagtggtgacggcggcaacatgtctgtggctttcgcggccccgaggcagcgaggcaagggggagatcactcccgctgcgattcagaagatgttggatgacaataaccatcttattcagtgtataatggactctcagaataaaggaaagacctcagagtgttctcagtatcagcagatgttgcacacaaacttggtataccttgctacaatagcagattctaatcaaaatatgcagtctcttttaccagcaccacccacacagaatatgcctatgggtcctggagggatgaatcagagcggccctcccccacctccacgctctcacaacatgccttcagatggaatggtaggtgggggtcctcctgcaccgcacatgcagaaccagatgaacggccagatgcctgggcctaaccatatgcctatgcagggacctggacccaatcaactcaatatgacaaacagttccatgaatatgccttcaagtagccatggatccatgggaggttacaaccattctgtgccatcatcacagagcatgccagtacagaatcagatgacaatgagtcagggacaaccaatgggaaactatggtcccagaccaaatatgagtatgcagccaaaccaaggtccaatgatgcatcagcagcctccttctcagcaatacaatatgccacagggaggcggacagcattaccaaggacagcagccacctatgggaatgatgggtcaagttaaccaaggcaatcatatgatgggtcagagacagattcctccctatagacctcctcaacagggcccaccacagcagtactcaggccaggaagactattacggggaccaatacagtcatggtggacaaggtcctccagaaggcatgaaccagcaatattaccctgatggaaattcacagtatggccaacagcaagatgcataccagggaccacctccacaacagggatatccaccccagcagcagcagtacccagggcagcaaggttacccaggacagcagcagggctacggtccttcacagggtggtccaggtcctcagtatcctaactacccacagggacaaggtcagcagtatggaggatatagaccaacacagcctggaccaccacagccaccccagcagaggccttatggatatgaccagggacagtatggaaattaccagcagtgaaaaagtacttacattccagtagccagtatctattagcagccatattgtcacctcagcactgtggacacctccctgtgaagagatccttccattccatctagtttttggaaaaaccttgtggataagtggctgtttcatcagtaagcagcctttgtggtttagttataaaaggctttagtagctcaaaaatactcttgatttcacatttctactctagatggcaacattggacagaaaatgcaatgacataaccaatttgtaatgattttggaactgtgtttcaaatggactgttacagactgaaaggtgtgaacagctttgtatgtttatgaagggtaagggaatttaatacttttccacagatttttttgtaaggggaagagggaaatgtacactttttacagcagcaatattttgtatattatgtttatttcatgtggtgaatatgcaaggcggtacactacgcactggacagcatcagaaatcctctgttaatgtggactggaacatggtagatgcttgattgttttggtctcaaaatggtgtgctataaagataaaggtgaggggaagacaaagcacaccatatgtccactgttctgttctcatagaggaaattcaaatcccttttatctattagataatcaagggcactgtgatacagttttgagtaaaaagacattttttaaaagccttccagttttgtggattaaacctttttataaagatcatttataatactgttttaaaatgtgaggcaataagaattactttgtgttggatctgaggaggctttggtaaaacagtttcatctaaatgaaagtggtaatcctcttctaaaatagcaataactgaaaatgaaagtgttaattttaccttgtttgagttatcagggaacttagtaagtaatatcaaagcattttataaatgatatcaaagaagagtcaacattgatccagtcattttattttgtaatattgagggataattggttattaaactgaatagttcaggagactttacaaacctttgtttcaactttcttatctggaaataatatcatttataaagggacacttttatgtttttcccttttttatgttggttgatataacacaaagagatatttaggaaaatgcttattgatgaggtttattctatctgtttttaaagcaccgaggttgcattctagataaccttgtttattagcatggcatattttaatcattatttgagactgtcctgtgcctgattattttagctaaattcagggagattgcgtggggcaggaaagcatgcattgaaaaatttctaaccacggttatttaagcataatctgaaaacatctagcccaaaggtaagttgctattttcatcacagttgcctatgcccagggaataagatgtattctttataattgaattggtttttcccacgtctaactggaaacaaaacagaaggggcgtcataaatttgaataagcagaacatactgttctcaacatactgtaatcaaaaggaggaatttcagtgggtctctgtgtgtgtatgagagagagagtgtgtgtttgtgtgtttcaaggtcagaacaggtttttttgtttttgttttttgttctttgtttttttttttgagatggagtcttgctcttgtcgcccaggctggagtgcagtggcgcaatctcagctcactgcaacctccgcctcccaggttcaagcagttctcctgcctcagcctcctgagtagctgggatgacaggcacccgccaccacacccagctaatttttgtacttttagtagagacgaggtttcgccatgttggccaggctggtctcgaactcctgacctcaggtgatccacccgcctcggccttccaaagtgctgggattacaggcgtgagccaccgtgcctggccagaataggttttttctttcaacttgatcagtagaaaatggacatcaagtttgaacagataaatcatggacagccttattgtgattgaaatgcttgtaggttctgtgccaattttccaccactgtgtactttgttgctatttaaaactgtatcaactctaacggaagaataaattatttgtgattttaaaaaa

SEQ ID NO: 6 - DNA, 4903 bp, Homo sapiens
gtcgtttgcggcggcgcaggcgcggtgcgggcggcggacgggcgggcgcttcgccgtttgaatggctgcgggcccgggccctcacctcacctgaggtccggccgcccaggggtgcgctatgccgtcgggaggtgaccagtcgccaccgcccccgcctccccctccggcggcggcagcctcggatgaggaggaggaggacgacggcgaggcggaagacgccgcgccgcctgccgagtcgcccacccctcagatccagcagcggttcgacgagctgtgcagccgcctcaacatggacgaggcggcgcgggccgaggcctgggacagctaccgcagcatgagcgaaagctacacgctggagggaaatgatcttcattggttagcatgtgccttatatgtggcttgcagaaaatctgttccaactgtaagcaaagggacagtggaaggaaactatgtatctttaactagaatcctgaaatgttcagagcagagcttaatcgaattttttaataagatgaagaagtgggaagacatggcaaatctacccccacatttcagagaacgtactgagagattagaaagaaacttcactgtttctgctgtaatttttaagaaatatgaacccatttttcaggacatctttaaataccctcaagaggagcaacctcgtcagcagcgaggaaggaaacagcggcgacagccctgtactgtgtctgaaattttccatttttgttgggtgctttttatatatgcaaaaggtaatttccccatgattagtgatgatttggtcaattcttatcacctgctgctgtgtgctttggacttagtttatggaaatgcacttcagtgttctaatcgtaaagaacttgtgaaccctaattttaaaggcttatctgaagattttcatgctaaagattctaaaccttcctctgaccccccttgtatcattgagaaactgtgttccttacatgatggcctagttttggaagcaaaggggataaaggaacatttctggaaaccctatattaggaaactttatgaaaaaaagctccttaagggaaaagaagaaaatctcactgggtttctagaacctgggaactttggagagagttttaaagccatcaataaggcctatgaggagtatgttttatctgttgggaatttagatgagcggatatttcttggagaggatgctgaggaggaaattgggactctctcaaggtgtctgaacgctggttcaggaacagagactgctgaaagggtgcagatgaaaaacatcttacagcagcattttgacaagtccaaagcacttagaatctccacaccactaactggtgttaggtacattaaggagaatagcccttgtgtgactccagtttctacagctacgcatagcttgagtcgtcttcacaccatgctgacaggcctcaggaatgcaccaagtgagaaactggaacagattctcaggacatgttccagagatccaacccaggctattgctaacagactgaaagaaatgtttgaaatatattctcagcatttccagccagacgaggatttcagtaattgtgctaaagaaattgccagcaaacattttcgttttgcggagatgctttactataaagtattagaatctgttattgagcaggaacaaaaaagactaggagacatggatttatctggtattctggaacaagatgcgttccacagatctctcttggcctgctgccttgaggtcgtcactttttcttataagcctcctgggaattttccatttattactgaaatatttgatgtgcctctttatcatttttataaggtgatagaagtattcattagagcagaagatggcctttgtagagaggtggtaaaacaccttaatcagattgaagaacagatcttagatcatttggcatggaaaccagagtctccactctgggaaaaaattagagacaatgaaaacagagttcctacatgtgaagaggtcatgccacctcagaacctggaaagggcagatgaaatttgcattgctggctcccctttgactcccagaagggtgactgaagttcgtgctgatactggaggacttggaaggagcataacatctccaaccacattatacgataggtacagctccccaccagccagcactaccagaaggcggctatttgttgagaatgatagcccctctgatggagggacgcctgggcgcatgcccccacagcccctagtcaatgctgtccctgtgcagaatgtatctggggagactgtttctgtcacaccagttcctggacagactttggtcaccatggcaaccgccactgtcacagccaacaatgggcaaacggtaaccattcctgtgcaaggtattgccaatgaaaatggagggataacattcttccctgtccaagtcaatgttggggggcaggcacaagctgtgacaggctccatccagcccctcagtgctcaggccctggctggaagtctgagctctcaacaggtgacaggaacaactttgcaagtccctggtcaagtggccattcaacagatttccccaggtggccaacagcagaagcaaggccagtctgtaaccagcagtagtaatagacccaggaagaccagctctttatcgcttttctttagaaaggtataccatttagcagctgtccgccttcgggatctctgtgccaaactagatatttcagatgaattgaggaaaaaaatctggacctgctttgaattctccataattcagtgtcctgaacttatgatggacagacatctggaccagttattaatgtgtgccatttatgtgatggcaaaggtcacaaaagaagataagtccttccagaacattatgcgttgttataggactcagccgcaggcccggagccaggtgtatagaagtgttttgataaaagggaaaagaaaaagaagaaattctggcagcagtgatagcagaagccatcagaattctccaacagaactaaacaaagatagaaccagtagagactccagtccagttatgaggtcaagcagcaccttgccagttccacagcccagcagtgctcctcccacacctactcgcctcacaggtgccaacagtgacatggaagaagaggagaggggagacctcattcagttctacaacaacatctacatcaaacagattaagacatttgccatgaagtactcacaggcaaatatggatgctcctccactctctccctatccatttgtaagaacaggctcccctcgccgaatacagttgtctcaaaatcatcctgtctacatttccccacataaaaatgaaacaatgctttctcctcgagaaaagattttctattacttcagcaacagtccttcaaagagactgagagaaattaatagtatgatacgcacaggagaaactcctactaaaaagagaggaattcttttggaagatggaagtgaatcacctgcaaaaagaatttgcccagaaaatcattctgccttattacgccgtctccaagatgtagctaatgaccgtggttcccactgaggttagtctcttgtattaaactcttcacaaaatctgtttagcagcagcctttaatgcatctagattatggagcttttttccttaatccagctgatgagttacagcctgttagtaacatgaggggacattttggtgagaaatgggacttaactccttccagtgtccttagaacattttaattcatcccaactgtctttttttccctaccattcagtgattactgtcaaggctgcttagaatccaaacttggatttttgactctggcaaagcttttagaaatactgcaagaaaatgatgtgtacccaaacgtgagcataggaggcttctgttgacgtactccaacagaagaactgtgtttcaagttcaatcctacctgttttgtggtcagctgtagtcctcataaaaagcaaaacaaaaattaggtattttgtcctaaaacacctggtaggagtgtgtgattttttgcattcctgacaaaggagagcacacccaggtttggaggtcctaggtcattagccctcgtctcccgttccctttgtgcacatcttccctctccccattcggtgtggtgcagtgtgaaaagtccttgattgttcgggtgtgcaatgtctgagtgaacctgtataagtggaggcactttagggctgtaaaatgcatgattttgtaacccagattttgctgtatatttgtgatagcactttctacaatgtgaactttattaaatacaaaacttccaggctaaacatccaatattttctttaatgcttttatatttttttaaaatgttaaaacccctatagccaccttttgggaatgttttaaattctccagttttttgttatatagggatcaaccagctaagaaaagattttaatcaagttgaattgaggggattaatatgaaaacttatgacctcttcctttaggagggagttatctaaaagaaatgtctattaaggtgatatatttaaaaatatttttgggtgttcctggcagtttaaaaaaattggttggagaatttaggtttttattagtaccatagtaccatttatacaaattagaaaatgttatttaacagctgaattatctatacatatctttattaatcactattgttccagcagttttcaagtcaaattaataatcttattagggagaaaattcaattgtaaattgaatcagtataaacaaagttactaggtaacttcatattgctgagagaaatatggaacttacattgttcaattagaatagtgttctgcaaaaatatttataaaacttctcaagatactgctactgtaattttatatgaagataagtgtatttttcaataaagcatttataaattaaaaaaaaaaaaaaaaaa

SEQ ID NO: 7 - DNA, 3324 bp, Homo sapiens
agtgccacaggcaaccctgcacgtgacgcttgcggaggaaggggagagagaggcgcgcgggagggcgtctagggaatcgaggtgccggctgctccttcctcacaatttggtttgtgctgcaaggggagggtccccatcatctggccccagtggtgtaaggagctgactgggattcagtcactgacttggagccgctcgggggaagtcccgcccagacaggcggtgggtgggaatgcctcacttcagtttgaagagggtccggatccaaaggggttaaaacgagcgacccccgatccccgaccacacttcccgcctccctaaaacgcacaccccgctagccatgggcagccgcgaccacctgttcaaagtgctggtggtgggggacgccgcagtgggcaagacgtcgctggtgcagcgatattcccaggacagcttcagcaaacactacaagtccacggtgggagtggattttgctctgaaggttctccagtggtctgactacgagatagtgcggcttcagctgtgggatattgcagggcaggagcgcttcacctctatgacacgattgtattatcgggatgcctctgcctgtgttattatgtttgacgttaccaatgccactaccttcagcaacagccagaggtggaaacaggacctagacagcaagctcacactacccaatggagagccggtgccctgcctgctcttggccaacaagtgtgatctgtccccttgggcagtgagccgggaccagattgaccggttcagtaaagagaacggtttcacaggttggacagaaacatcagtcaaggagaacaaaaatattaatgaggctatgagagtcctcattgaaaagatgatgagaaattccacagaagatatcatgtctttgtccacccaaggggactacatcaatctacaaaccaagtcctccagctggtcctgctgctagtagtgtttggcttattttccatcccagttctgggaggtcttttaagtctcttccctttggttgcccacctgacaattttattaagtacatttgaattgtctcctgactactgtccagtaaggaggcccattgtcacttagaaaagacacctggaacccatgtgcatttctgcatctcctggattagcctttcacatgttgctggctcacattagtgccagttagtgccttcggtgtaagatcttctcatcagccctcaatttgtgatccggaattttatgagaaggattagaaatcagcacctgcgttttagagatcataattctcacctacttctgagcttatttttccatttgatattcattgatatcatgacttccaattgagaggaaaatgagatcaaatgtcatttcccaaatttcttgtaggccgttgtttcagattctttctgtcttggaatgtaaacatctgattctggaatgcagaaggagggggtctgggcatctgtggatttttggctactagaagtgtcccagaagtcactgtatttttgaaacttctaacgtcataattaagtttctcttgtcttggcatcaagaatagtcaagttttttggccgggcatggtggctcatgcctgtaatcccagcacttggggaggccaaggcaggcggatcacatgaggccaggaattcgagaccaacctggtcagcatggcaaaaccccgtctctactaaaagtacaaaaattagccaggcgtgatggcacgtgtctgtaatcccagctactctggagactgaggtgggagaatcgcttgagactgggaggcagaggttgcagtgaaccgagatcatgccaccgcacttcagcctgggtgacagagaaggactccgtctcaaaaaaaaaagaaaaaagaatagtcatttttaaactacctatctcatgcaatgaaagcattttcttccacaaagagcttaatcctcatgataggattgcctagtgtctcccatttgcaggtttctgggttgatgtcttaatgcataatactgcaagtgacatcagctggctgtgatgcttcgaaataggtctgctcctcacagctttgggaatctgaatggaagaagaaaagagagaagttaacaacctccactggggcaactttgtgaacacgtaggcacttagtcataggaaacatattatgtgcaggtcctagcctgggggaggaaagtagatagacagaaaatcattaggtaatttaagtactaaattgggcagggctttttagtatcaaatcactactagaccatttaatttgttaaattatctctaggatggtgatttataacctacccaaagttatcgatattcttactaaactctgaggcctgaagttctgtgatagaccttaaataagtgtcctaagtcagtggttcccaaatctggctggtcgggaatacctgggaagtttgttaaaattttttaaaaatgttttaagatttttgggtcctgagccagccgtggtggctcacacctgtaatcccagcactttgggaggctgaggcaggtggatcgcctgaggtcaggagttcaagatcaacctggccaacatactgaaaccccgtctctactaaaaataagaaaaattagctgggcgtggtggcgggcacctgtaatcccagctacttgggagactgaggcaggagaatcgcttgaacctgggagttagaggttgcagtgagctgagatcacaccattgcgcttcagcctgggcaacaagagtgaaactccatctccaaaaaaaaaaaacaaaaagaaaaagatttttgggtcccgacctcaaacctactgaatcagaatttctagggatgaagcctaggaatgtgttgttgttttcagagcttccctggtgattgtgataagcctggtttggaaaccattgctggagaactttgtaaagatacagagacccagaccttttgtatttacatttaaatacaaatacaaatcctgggtttctatatattctgttagcttttcaggtgattctgctacacagacgttgaaaaccactgccctaagaaagagatcagaggccacatatcagagagaaaagggaccaaaccttcggtggtttgttgtgtgtcgttttaatgccaattattttaacttgcacagtcttctgaaaccttgtattaatagttctcttttgtattaccattttcaggtagggttttgatcactatgattctgaagataatagtgaaatagtgaatttcattgatatgaagagataattgattttcattcattggtttgaacacctgcaaaatcacaaataaatgagaactaagtcttgtaaaaaaaaaaaaaaaaa

SEQ ID NO: 8 - DNA, 3466 bp, Homo sapiens
aattacggccggattccggagtcctttccagctccctcttcggccgggtttcccgccgaatacaaaggcgcactgtgaactggctctttctttccgccaatcatttccgccagccattcatcaccgattttcttcatcttcccctccctcttccgtcccgcagtccccgacctgttagctctcggttagttaagggactcgggtccttccgaactgcgcatgcgccaccgcgtctgcagggggagaagcgggcaggggcgcaggcgcagtagtgtgatcccctggccagtccctaagcacgtgggttgggttgtcctgcttggctgcggagggagtggaacctcgatattggtggtgtccatcgtgggcagcggactaataaaggccatggcgccagcagaaatcctgaacgggaaggagatctccgcgcaaataagggcgagactgaaaaatcaagtcactcagttgaaggagcaagtacctggtttcacaccacgcctggcaatattacaggttggcaacagagatgattccaatctttatataaatgtgaagctgaaggctgctgaagagattgggatcaaagccactcacattaagttaccaagaacaaccacagaatctgaggtgatgaagtacattacatctttgaatgaagactctactgtacatgggttcttagtgcagctacctttagattcagagaattccattaacactgaagaagtgatcaatgctattgcacccgagaaggatgtggatggattgactagcatcaatgctgggaaacttgctagaggtgacctcaatgactgtttcattccttgtacgcctaagggatgcttggaactcatcaaagagacaggggtgccgattgccggaaggcatgctgtggtggttgggcgcagtaaaatagttggggccccgatgcatgacttgcttctgtggaacaatgccacagtgaccacctgccactccaagactgcccatctggatgaggaggtaaataaaggtgacatcctggtggttgcaactggtcagcctgaaatggttaaaggggagtggatcaaacctggggcaatagtcatcgactgtggaatcaattatgtcccagatgataaaaaaccaaatgggagaaaagttgtgggtgatgtggcatacgacgaggccaaagagagggcgagcttcatcactcctgttcctggcggcgtagggcccatgacagttgcaatgctcatgcagagcacagtagagagtgccaagcgtttcctggagaaatttaagccaggaaagtggatgattcagtataacaaccttaacctcaagacacctgttccaagtgacattgatatatcacgatcttgtaaaccgaagcccattggtaagctggctcgagaaattggtctgctgtctgaagaggtagaattatatggtgaaacaaaggccaaagttctgctgtcagcactagaacgcctgaagcaccggcctgatgggaaatacgtggtggtgactggaataactccaacacccctgggagaagggaaaagcacaactacaatcgggctagtgcaagcccttggtgcccatctctaccagaatgtctttgcgtgtgtgcgacagccttctcagggccccacctttggaataaaaggtggcgctgcaggaggcggctactcccaggtcattcctatggaagagtttaatctccacctcacaggtgacatccatgccatcactgcagctaataacctcgttgctgcggccattgatgctcggatatttcatgaactgacccagacagacaaggctctctttaatcgtttggtgccatcagtaaatggagtgagaaggttctctgacatccaaatccgaaggttaaagagactaggcattgaaaagactgaccctaccacactgacagatgaagagataaacagatttgcaagattggacattgatccagaaaccataacttggcaaagagtgttggataccaatgatagattcctgaggaagatcacgattggacaggctccaacggagaagggtcacacacggacggcccagtttgatatctctgtggccagtgaaattatggctgtcctggctctcaccacttctctagaagacatgagagagagactgggcaaaatggtggtggcatccagtaagaaaggagagcccgtcagtgccgaagatctgggggtgagtggtgcactgacagtgcttatgaaggacgcaatcaagcccaatctcatgcagacactggagggcactccagtgtttgtccatgctggcccgtttgccaacatcgcacatggcaattcctccatcattgcagaccggatcgcactcaagcttgttggcccagaagggtttgtagtgacggaagcaggatttggagcagacattggaatggaaaagttttttaacatcaaatgccggtattccggcctctgcccccacgtggtggtgcttgttgccactgtcagggctctcaagatgcacgggggcggccccacggtcactgctggactgcctcttcccaaggcttacatacaggagaacctggagctggttgaaaaaggcttcagtaacttgaagaaacaaattgaaaatgccagaatgtttggaattccagtagtagtggccgtgaatgcattcaagacggatacagagtctgagctggacctcatcagccgcctttccagagaacatggggcttttgatgccgtgaagtgcactcactgggcagaagggggcaagggtgccttagccctggctcaggccgtccagagagcagcacaagcacccagcagcttccagctcctttatgacctcaagctcccagttgaggataaaatcaggatcattgcacagaagatctatggagcagatgacattgaattacttcccgaagctcaacacaaagctgaagtctacacgaagcagggctttgggaatctccccatctgcatggctaaaacacacttgtctttgtctcacaacccagagcaaaaaggtgtccctacaggcttcattctgcccattcgcgacatccgcgccagcgttggggctggttttctgtaccccttagtaggaacgatgagcacaatgcctggactccccacccggccctgtttttatgatattgatttggaccctgaaacagaacaggtgaatggattattctaaacagatcaccatccatcttcaagaagctactttgaaagtctggccagtgtctattcaggcccactgggagttaggaagtataagtaagccaagagaagtcagcccctgcccagaagatctgaaactaatagtaggagtttccccagaagtcattttcagccttaattctcatcatgtataaattaacataaatcatgcatgtctgtttactttagtgacgttccacagaataaaaggaaacaagtttgccatcaaaaaaaaaaaaaaaaaa

SEQ ID NO: 9 - DNA, 2543 bp, Homo sapiens
caatttttaagaaatgatcatctagggaaaatgttttaaaatcttttaaaatatctttttgttatactttaaaactgccttcagctgcctattggtaaataaggtctggatatcagtacatgacaacatatactaatgaattgaactgtgaattaggaatgaaaattgcctagaaatttttttatgagttattaggattaaagtaagaatttcctttgctgggctgtgtaggcactaagattccgtggttgttggaggggttaaatttggatctactagtgcatatattttaccgaggattcataaaaacacgacagtggagcctgggtcaatccattaccatgtacaagcagtgaccaatagaaaaggaagaagaagatccaagaaggctgaggtaagcagtcttctgaagccctgtcaaagtggaaacaatagatcaaaggtgttcatggtgactgggacgagtaggtttcactgtttctcataggagacttgacagcttaaagtaaaaacaaattattttcgtcaaagtttttttttttctcttaactgatttttagcaaacctcagactgagacacaggactcaacggtgtattcctggaaggcaaggtgctataatggcaggcacaatctgtttcatcatgtgggtgttattcataacagacactgtgtggtctagaagtgtgaggcaggtctatgaagtacatgattcagatgattggactattcatgacttcgagtgtcccatggaatgtttctgcccacccagttttcctactgctttatattgtgaaaatagaggtctcaaagaaattcctgctattccttcaagaatttggtatctttatcttcaaaacaacctgatagaaaccattcctgaaaagccatttgagaatgccacccagctaagatggataaatctaaacaagaacaaaataaccaactacggaattgaaaaaggagccctaagccagctgaagaagttgctcttcttatttctggaagataatgagctagaggaggtaccttctccattgccaagaagtttagaacaattacaattagctagaaataaggtgtccagaattcctcaagggacctttagcaatctggagaacctgacccttcttgacctacagaacaacaaattagtggacaatgcctttcaaagagacacttttaaaggactcaagaatctcatgcagctaaacatggccaagaatgccctgaggaatatgcctccaagattaccagccaatacaatgcagttgtttttagacaacaattccattgaaggaataccagaaaattattttaatgtgattcctaaagtggcctttttgagactaaatcacaacaaactgtcagatgagggtctcccatcaagaggatttgatgtatcatcaattctagatcttcaactgtcgcacaatcaactcacaaaggttccccgaatcagtgctcatctgcagcaccttcaccttgatcataacaaaattaaaagtgtgaatgtctctgtaatatgtcccagcccatccatgctgcctgcagaacgagattccttcagttatggacctcatcttcgctacctccgtctggatggaaatgaaatcaaaccaccaattccaatggctttaatgacctgcttcagacttctgcaggctgtcattatttaaacacattctcaccaaatctaaaattagtttaatgagctgtttctgacatgaaatgtggttaccattaataggtttaggacacaagtcatattccccattgctctcggccaccattttcatttgtgcagtgtattttttctattcaaagatgcttttgccagttacatgcatcacagcctgcattaatttgcttttcttttaattaataaaacagacacagagttaagatagtttatcaactcaaagatagttttattttggtctcttccatagcttattaacactaaagaaaacaattacattcttatacaataaaaaggacacatttgtgtatgtttaaaattacttatgcagataccgtaatttacagtataaatgtaataatcaaacaggaggataacgacctgaagaaaatgagaaaataaaatatttcttaattgtaatcataataaaacaattgcagattgctaagtagctgttggtggtacaagtttgatatttagcttgagaatgggtatggaatgaatcaaatacttcatcactaaagaattcttgttatttaatatcaaataatgaaacagcagtcacttctagttcattaccatttcttgtagctgttgtttttagatatagtccactgcattcttataagttctaaattttatgtttattataatgcatttcttgtggaatcttagtgttttaacatttgaaatccacttaaactgcaaattgttctttatatttgattgtgttttttggattttctaaataaaaatattgaatgctttctaacaaaacagaatatttcaaagaaattagcaagcttcattaaagaacatgttttaataaaaaaaaaaaaaaa

SEQ ID NO: 10 - DNA, 3435 bp, Homo sapiens
acagcaactatgaaataatcgtagtatgagaggcagagatcggggcgagacaatggggatgtgggcgcgggagccccgttccggcttagcagcacctcccagccccgcagaataaaaccgatcgcgccccctccgcgcgcgccctcccccgagtgcggagcgggaggaggcggcggcggccgaggaggaggaggaggaggccccggaggaggaggcgttggaggtcgaggcggaggcggaggaggaggaggccgaggcgccggaggaggccgaggcgccggagcaggaggaggccggccggaggcggcatgagacgagcgtggcggccgcggctgctcggggccgcgctggttgcccattgacagcggcgtctgcagctcgcttcaagatggccgcttggctcgcattcattttctgctgaacgacttttaactttcattgtcttttccgcccgcttcgatcgcctcgcgccggctgctctttccgggattttttatcaagcagaaatgcatcgaacaacgagaatcaagatcactgagctaaatccccacctgatgtgtgtgctttgtggagggtacttcattgatgccacaaccataatagaatgtctacattccttctgtaaaacgtgtattgttcgttacctggagaccagcaagtattgtcctatttgtgatgtccaagttcacaagaccagaccactactgaatataaggtcagataaaactctccaagatattgtatacaaattagttccagggcttttcaaaaatgaaatgaagagaagaagggatttttatgcagctcatccttctgctgatgctgccaatggctctaatgaagatagaggagaggttgcagatgaagataagagaattataactgatgatgagataataagcttatccattgaattctttgaccagaacagattggatcggaaagtaaacaaagacaaagagaaatctaaggaggaggtgaatgataaaagatacttacgatgcccagcagcaatgactgtgatgcacttaagaaagtttctcagaagtaaaatggacatacctaatactttccagattgatgtcatgtatgaggaggaacctttaaaggattattatacactaatggatattgcctacatttatacctggagaaggaatggtccacttccattgaaatacagagttcgacctacttgtaaaagaatgaagatcagtcaccagagagatggactgacaaatgctggagaactggaaagtgactctgggagtgacaaggccaacagcccagcaggaggtattccctccacctcttcttgtttgcctagccccagtactccagtgcagtctcctcatccacagtttcctcacatttccagtactatgaatggaaccagcaacagccccagcggtaaccaccaatcttcttttgccaatagacctcgaaaatcatcagtaaatgggtcatcagcaacttcttctggttgatacctgagactgttaaggaaaaaaattttaaacccctgatttatatagatatcttcatgccattacagctttctagatgctaatacatgtgactatcgtccaatttgctttcttttgtagtgacattaaatttggctataaaagatggactacatgtgatactcctatggacgttaattgaaaagaaagattgttgttataaagaattggtttcttggaaagcaggcaagactttttctctgtgttaggaaagatgggaaatggtttctgtaaccattgtttggatttggaagtactctgcagtggacataagcattgggccatagtttgttaatctcaactaacgcctacattacattctccttgatcgttcttgttattacgctgttttgtgaacctgtagaaaacaagtgctttttatcttgaaattcaaccaacggaaagaatatgcatagaataatgcattctatgtagccatgtcactgtgaataacgatttcttgcatatttagccattttgattcctgtttgatttatacttctctgttgctacgcaaaaccgatcaaagaaaagtgaacttcagttttacaatctgtatgcctaaaagcgggtactaccgtttattttactgacttgtttaaatgattcgcttttgtaagaatcagatggcattatgcttgttgtacaatgccatattggtatatgacataacaggaaacagtattgtatgatatatttataaatgctataaagaaatattgtgtttcatgcattcagaaatgattgttaaaattctcccaactggttcgacctttgcagatacccataacctatgttgagccttgcttaccagcaaagaatatttttaatgtggatatctaattctaaagtctgttccattagaagcaattggcacatctttctatactttatatacttttctccagtaatacatgtttactttaaagattgttgcagtgaagaaaaacctttaactgagaaatatggaaaccgtcttaattttccattggctatgatggaattaatattgtattttaaaaatgcatattgatcactataattctaaaacaattttttaaataaaccagcaggttgctaaaagaaggcattttatctaaagttattttaataggtggtatagcagtaattttaaatttaagagttgcttttacagttaacaatggaatatgccttctctgctatgtctgaaaatagaagctatttattatgagcttctacaggtatttttaaatagagcaagcatgttgaatttaaaatatgaataaccccacccaacaattttcagtttattttttgctttggtcgaacttggtgtgtgttcatcacccatcagttatttgtgagggtgtttattctatatgaatattgtttcatgtttgtatgggaaaattgtagctaaacatttcattgtccccagtctgcaaaagaagcacaattctattgctttgtcttgcttatagtcattaaatcattacttttacatatattgctgttacttctgctttctttaaaaatatagtaaaggatgttttatgaagtcacaagatacatatatttttattttgacctaaatttgtacagtcccattgtaagtgttgtttctaattatagatgtaaaatgaaatttcatttgtaattggaaaaaatccaataaaaaggatattcatttagaaaatagctaagatctttaataaaaatttgatatgaaaagcacaatgtgcagaagttatggaaaacctatagaggattacaacaggtaaacgttaaagagaatacattgctgacttatagtgatgtggctaagaagtacatgctttgttgtaaaattgcttgaaagcccattgaaagatgtatctgtttatttacagtctttgaagtaaaagttaccaatgtttgccaataaaaa

SEQ ID NO: 11 - DNA, 4880 bp, Homo sapiens
attggtccttttatggttcagacttccttcttcatcgactctccctaattctcttcactgttcatccccagcaacttttcttcccaagcatttcctgcgggcttttattaccccaggagtgccaatcaaggcgtggaacagataaagctgtgtagcaactaacgcgtgactcttgtgagtgtggagggcaaacgaagctccctgaaacctccgctctgctctcctgccttacgccgcgcggcccctgttctgaggtttaatacttgcttcacagataagcgctgcggccacgttccggtgcccaccttctccttggctagggcacgacgccagcccagggggcgcggccctgagctgctgcgagagacagctgaaagcgccggcccgacggccttggctccctcggtgcgctgggcgcagagcactcggaccctgggcgcccactgctcgagtacctgcgcggctcctagccaggcttagcccaggcgcgggctgagagtcagttcgccaggtgggcctggagccatgggctgggtgggcgggcggcgccgggattctgcgtcaccacctgggcggagccgttctgctgctgacgacatcaacccggcacctgccaacatggaaggtggcggcggcagcgtcgctgtagctggcctcggagctcgaggctctggagcggctgcagctacagtccgggaacttctgcaggacgggtgttatagtgactttttaaacgaagactttgatgtaaagacttatacttctcaatctattcatcaagctgtaattgctgaacaactagcaaaacttgcccaaggaatcagtcagttggacagagaactacacttacaggttgttgcaagacatgaagatttactggcacaagcaactgggattgagtcgttggaaggtgttcttcagatgatgcagacgagaattggggctttacagggagctgttgataggataaaagcaaaaattgttgaaccatacaataagatagttgcccggactgcacaactagcaagacttcaggttgcctgtgatttgcttcggaggattattcgtatcttgaatctcagtaagagactccaaggacaactgcaagggggaagtagagagataacaaaagctgctcagagtctcaatgaacttgattatctttctcaaggaatagatctttctggaatagaagtgatagaaaatgatctactttttattgcaagagcccgacttgaagtggaaaatcaagctaagcgcctactagagcagggtttggagactcagaatccaactcaagtcggaacagctcttcaggttttctataatcttggaactttgaaggatactattaccagtgttgtggatggatattgtgctactttagaagaaaatatcaacagtgcattagacataaaagttttgactcagccttcccagtcagctgtgagagggggacctggacgatctaccatgccaaccccaggaaatactgcagctttgcgtgcctcattctggaccaatatggagaaacttatggatcatatttatgctgtttgtggacaggtacaacatctacaaaaagtattggccaagaagagagatcctgtttctcacatttgtttcattgaagaaatagttaaggatggacaaccggaaattttctacacattttggaattcagttactcaggcactttcttctcaatttcatatggcaacaaactcttcgatgtttttgaagcaggcatttgaaggagaataccctaaattattacgtctttataatgacttatggaagcgtcttcaacaatacagtcagcatatccaagggaattttaatgcaagtggaactacagacctctatgttgacctacaacacatggaagatgatgcacaagatatattcataccaaaaaagccagattatgatccagaaaaggctttgaaagactcactacaaccctatgaggctgcttatctatcaaaatccttatctcgactcttcgatcctatcaacttggtttttcccccgggtggtcgtaatcctccttcctctgatgaacttgatggtattattaaaactatagcaagtgaactaaatgttgctgctgttgatacaaacctcacattagctgtgtcaaaaaatgtggcaaagaccatccagttatacagtgtaaaatcagagcagcttctctccacacaaggagatgcaagtcaggtgattgggcctcttactgaaggacagagaagaaatgtggcagtagtgaattcattgtataagttgcaccaatcagtaacaaaggttgtttccagtcagagctcattcccactggcagctgagcaaactataatttcagctctaaaggctattcatgctcttatggaaaatgctgtgcaacccttactcacttctgtgggagatgctatagaggccataatcatcaccatgcatcaagaagacttttctgggtcattatccagctcaggaaaacctgatgttccttgttctctgtacatgaaggagctacaaggtttcattgccagagttatgagtgactattttaaacactttgaatgcttggattttgtctttgacaacactgaggctattgcccaaagagctgttgaactttttatccgccatgccagtctcataagacctcttggtgaaggtgggaaaatgcgacttgctgctgattttgcacagatggagttggctgtgggtccattctgtagacgagtatctgatttaggaaagtcctatcggatgctgagatcattcagacctctgctcttccaggcaagtgaacatgtagccagtagtcctgcattgggggatgtgattccgttcagcatcattattcagtttttgttcacgagagcacccgctgaactgaaatctcctttccagagggcagagtggtcccacacacgcttctctcagtggctggatgaccatccatctgaaaaggacaggctcctcctcatcaggggagccctggaagcttatgttcaatcagtgagaagtagagaaggcaaagaatttgcaccagtttatcccataatggttcagctgcttcaaaaggctatgtctgctcttcagtaatgacatgaaatctttgttcatctccactttgtgctaacccattcatagttggcagttaaacacatactccaaaagactgctactatctactattttaagaatgtaattgattgttcggtatttcctatcgacgtttatttacctctttagcacttatactttagcataaaaaatgttgagttatcaccacctttcaattccatggacctgatttttccagaaagatgttttcctctttcagatttttgtacaaggctaaaatgtctttcccatccataaccaagtcctcctatgggtacataaacccaaagtccccacttcttttaaagggatatgatcaagttataacatgtaccctgcttcccccaaccctgccttcttcactaaataagcatgtagctcagtggtttccaaatttggctgcacattcataccaatcaccaggggatttttttaaaatcctgatgcccaacttgcactccacattaattaacatgtctaggagtgggagcctgacagacaccactattaaaaaaaaaaaaaatccccaaaatgattccaatagacaacaaagttgaggaaccactggcacatcccaagctaagatacaaggttaaatggcctttttaagtatgtcatactggatctttaaataaagcaaggcttttgttacactttgtcatgttattaaaagcagacctttgggctgtttaaccgtgtaacaaaaatgccacgtgaaaaataaaaatttttattgtatagcaattctctataaatagtagataatctaagtccttattttctgatggctcttgttccactattaacattgtttttaatttttaaaatcctatcagcagcctcctaattagcagtgttaggaatttgccttatgttttccatctcatctcctgaacctgttattctgagaactcataaataaaattcagccaagattaatcaatcaatcaatcaatcagtagacgtcagagataagcaacttttttttccatttatttgaaataatatacaagaatatagtgtgcaaaatttaggggcaacatcatgaagaagtgtgatgaactgaaatcaattttacagctgtgattcctaaccagaaacaccacttggtaagtaacatgaaaagccatgattgtagctcagagcaaaagcagctgtattgccaatgtatttgctctaattttagacagttggaggaaaattaaagtgcaagtgttgcactgcagtaagtgcaaagtctgcccccaccataaagattatgatgacaaaatatatttaagatacatggctgaggagatacagaacacgaagttaaaaaatacaatggtgttgactgtttgttaatagcaccattacagtgactatatcccaatgctattaactaactttaccttggcctgggctaggtctccattggaaataataacggatggctgctgccacctggtgcatcaactcagcatgccatggtaactgcagcaggtggctcttaggctgggacaccaacaccagccacctgccccatgttctggtagcctcttggcactgaccagactaggagcgcctatgtttggcacagatgctgagccatgtggtggttactagaggaaaggccgactcccagcttctgcttcatttcatcatgtgtacaatacagattaataagcagtaaggagccaatgtataaacagcagttacatattttttaaattcccctttcgtacaatttaaaataaaacatgtatacactt

SEQ ID NO: 12 - DNA, 7783 bp, Homo sapiens
aatacttgttgcaataattgcccacgatagctgctcaaacaagagagttggaattcatctgtaaaaatcactacatgtaacgtaggagacaagaaaaatattaatgacagaagatctgcgaacatgatgcacgtgaataattttccctttagaaggcattcctggatatgttttgatgtggacaatggcacatctgcgggacggagtcccttggatcccatgaccagcccaggatccgggctaattctccaagcaaattttgtccacagtcaacgacgggagtccttcctgtatcgatccgacagcgattatgacctctctccaaagtctatgtcccggaactcctccattgccagtgatatacacggagatgacttgattgtgactccatttgctcaggtcttggccagtctgcgaactgtacgaaacaactttgctgcattaactaatttgcaagatcgagcacctagcaaaagatcacccatgtgcaaccaaccatccatcaacaaagccaccataacagaggaggcctaccagaaactggccagcgagaccctggaggagctggactggtgtctggaccagctagagaccctacagaccaggcactccgtcagtgagatggcctccaacaagtttaaaaggatgcttaatcgggagctcacccatctctctgaaatgagtcggtctggaaatcaagtgtcagagtttatatcaaacacattcttagataagcaacatgaagtggaaattccttctccaactcagaaggaaaaggagaaaaagaaaagaccaatgtctcagatcagtggagtcaagaaattgatgcacagctctagtctgactaattcaagtatcccaaggtttggagttaaaactgaacaagaagatgtccttgccaaggaactagaagatgtgaacaaatggggtcttcatgttttcagaatagcagagttgtctggtaaccggcccttgactgttatcatgcacaccatttttcaggaacgggatttattaaaaacatttaaaattccagtagatactttaattacatatcttatgactctcgaagaccattaccatgctgatgtggcctatcacaacaatatccatgctgcagatgttgtccagtctactcatgtgctattatctacacctgctttggaggctgtgtttacagatttggagattcttgcagcaatttttgccagtgcaatacatgatgtagatcatcctggtgtgtccaatcaatttctgatcaatacaaactctgaacttgccttgatgtacaatgattcctcagtcttagagaaccatcatttggctgtgggctttaaattgcttcaggaagaaaactgtgacattttccagaatttgaccaaaaaacaaagacaatctttaaggaaaatggtcattgacatcgtacttgcaacagatatgtcaaaacacatgaatctactggctgatttgaagactatggttgaaactaagaaagtgacaagctctggagttcttcttcttgataattattccgataggattcaggttcttcagaatatggtgcactgtgcagatctgagcaacccaacaaagcctctccagctgtaccgccagtggacggaccggataatggaggagttcttccgccaaggagaccgagagagggaacgtggcatggagataagccccatgtgtgacaagcacaatgcttccgtggaaaaatcacaggtgggcttcatagactatattgttcatcccctctgggagacatgggcagacctcgtccaccctgacgcccaggatattttggacactttggaggacaatcgtgaatggtaccagagcacaatccctcagagcccctctcctgcacctgatgacccagaggagggccggcagggtcaaactgagaaattccagtttgaactaactttagaggaagatggtgagtcagacacggaaaaggacagtggcagtcaagtggaagaagacactagctgcagtgactccaagactctttgtactcaagactcagagtctactgaaattccccttgatgaacaggttgaagaggaggcagtaggggaagaagaggaaagccagcctgaagcctgtgtcatagatgatcgttctcctgacacgtaacagtgcaaaaactttcatgccttttttttttttaagtagaaaaattgtttccaaagtgcatgtcacatgccacaaccacggtcacacctcactgtcatctgccaggacgtttgttgaacaaaactgaccttgactactcagtccagcgctcaggaatatcgtaaccagttttttcacctccatgtcatccgagcaaggtggacatcttcacgaacagcgtttttaacaagatttcagcttggtagagctgacaaagcagataaaatctactccaaattattttcaagagagtgtgactcatcaggcagcccaaaagtttattggacttggggtttctattcctttttatttgtttgcaatattttcagaagaaaggcattgcacagagtgaacttaatggacgaagcaacaaatatgtcaagaacaggacatagcacgaatctgttaccagtaggaggaggatgagccacagaaattgcataattttctaatttcaagtcttcctgatacatgactgaatagtgtggttcagtgagctgcactgacctctacattttgtatgatatgtaaaacagattttttgtagagcttacttttattattaaatgtattgaggtattatatttaaaaaaaactatgttcagaacttcatctgccactggttatttttttctaaggagtaacttgcaagttttcagtacaaatctgtgctacactggataaaaatctaatttatgaattttacttgcaccttatagttcatagcaattaactgatttgtagtgattcattgtttgttttatataccaatgacttccatattttaaaagagaaaaacaactttatgttgcaggaaaccctttttgtaagtctttattatttactttgcattttgtttcactctttccagataagcagagttgctcttcaccagtgtttttcttcatgtgcaaagtgactatttgttctataatacttttatgtgtgttatatcaaatgtgtcttaagcttcatgcaaactcagtcatcagttcgtgttgtctgaagcaagtgggagatatataaatacccagtagctaaaatggtcagtcttttttagatgttttcctacttagtatctcctaataacgttttgctgtgtcactagatgttcatttcacaagtgcatgtctttctaataatccacacatttcatgctctaataatccacacatttcatgctcatttttattgtttttacagccagttatagtaagaaaaaggtttttccccttgtgctgctttataatttagcgtgtgtctgaaccttatccatgtttgctagatgaggtcttgtcaaatatatcactaccattgtcaccggtgaaaagaaacaggtagttaagttagggttaacattcatttcaaccacgaggttgtatatcatgactagcttttactcttggtttacagagaaaagttaaacagccaactaggcagtttttaagaatattaacaatatattaacaaacaccaatacaactaatcctatttggttttaatgatttcaccatgggattaagaactatatcaggaacatccctgagaaacggttttaagtgtagcaactactcttccttaatggacagccacataacgtgtaggaagtcctttatcacttatcctcgatccataagcatatcttgcagaggggaactacttctttaaacacatggagggaaagaagatgatgccactggcaccagagggttagtactgtgatgcatcctaaaatatttattatattggtaaaaattctggttaaataaaaaattagagatcactcttggctgatttcagcaccaggaactgtattacagttttagagattaattcctagtgtttacctgattatagcagttggcatcatggggcatttaattctgactttatccccacgtcagccttaataaagtcttctttaccttctctatgaagactttaaagcccaaataatcatttttcacattgatattcaagaattgagatagatagaagccaaagtgggtatctgacaagtggaaaatcaaacgtttaagaagaattacaactctgaaaagcatttatatgtggaacttctcaaggagcctcctggggactggaaagtaagtcatcagccaggcaaatgactcatgctgaagagagtccccatttcagtcccctgagatctagctgatgcttagatcctttgaaataaaaattatgtctttataactctgatcttttacataaagcagaagaggaatcaactagttaattgcaaggtttctactctgtttcctctgtaaagatcagatggtaatctttcaaataagaaaaaaataaagacgtatgtttgaccaagtagtttcacaagaatatttgggaacttgtttcttttaattttatttgtccctgagtgaagtctagaaagaaaggtaaagagtctagagtttattcctctttccaaaacattctcattcctctcctccctacacttagtatttcccccacagagtgcctagaatcttaataatgaataaaataaaaagcagcaatatgtcattaacaaatccagacctgaaagggtaaagggtttataactgcactaataaagagaggctctttttttttcttccagtttgttggtttttaatggtaccgtgttgtaaagatacccactaatggacaatcaaattgcagaaaaggctcaatatccaagagacagggactaatgcactgtacaatctgcttatccttgcccttctctcttgccaaagtgtgcttcagaaatatatactgctttaaaaaagaataaaagaatatccttttacaagtggctttacatttcctaaaatgccataagaaaatgcaatatctgggtactgtatggggaaaaaaatgtccaagtttgtgtaaaaccagtgcatttcagcttgcaagttactgaacacaataatgctgttttaattttgttttatatcagttaaaattcacaataatgtagatagaacaaattacagacaaggaaagaaaaaacttgaatgaaatggattttacagaaagctttatgataatttttgaatgcattatttattttttgtgccatgcattttttttctcaccaaatgaccttacctgtaatacagtcttgtttgtctgtttacaaccatgtatttattgcaatgtacatactgtaatgttaattgtaaattatctgttcttattaaaacatcatcccatgatgggatggtgttgatatatttggaaactcttggtgagagaatgaatggtgtgtatacatactctgtacatttttcttttctcctgtaatatagtcttgtcaccttagagcttgtttatggaagattcaagaaaactataaaatacttaaagatatataaatttaaaaaaacatagctgcaggtctttggtcccagggctgtgccttaactttaaccaatattttcttctgttttgctgcatttgaaaggtaacagtggagctagggctgggcattttacatccaggcttttaattgattagaattctgccaataggtggattttacaaaaccacagacaacctctgaaagattctgagacccttttgagacagaagctcttaagtacttcttgccagggagcagcactgcatgtgtgatggttgtttgccatctgttgatcaggaactacttcagctacttgcatttgattatttcctttttttttttttttaactcggaaacacaactggggaaatatattctttcccagtgattataaacaatctttttcttttttttaagtccttttggcttctagagctcataggaaaatggacttgatttgaaattggagccagagtttactcgtgttggttatctattcatcagcttcctgacatgttaagagaatacattaaagagaaaatactgttttttaatcctaaaatttttcttccactaagataaaccaaatgtccttacatatatgtaaacccatctatttaaacgcaaaggtgggttgatgtcagtttacatagcagaaagcattcactatcctctaagatttgtttctgcaaaactttcattgctttagaattttaaaatttcaccttgtacaatggccagcccctaaagcaggaaacatttataatggattatatggaaacatcctcccagtacttgcccagcccttgaatcatgtggcttttcagtgaaaggaaagattctttttctaggaaaaatgagcctattttattttattttattttattttttgacacaaactgtagattttagcagccctggcccaaaggaatttgattacttttgttttaaacagtacaaaggggacactataattacaaaaacatccttaactgatttgagttgtttttatttctttggatatattttcagagtggtaaattgtgtgtgagaattacaaatgattattcttttagtggtttcttagcctctcttacagcccacggggatagtactgtacatcaataccttcatatgaaatttttatatgcaatgaaaataaaagcatgggttgattctgcctatttatgactcaatcttttacaaataaaagattattcattttaaattatagttcaatcagcatgtctcttaggatactgaacgtggttgaaatgaaaggatagtgacatcataagttagtactgatattcataaccaaataaagccaacttgagtaattttgctacattaaaaattaccaaaattacttagatggcctataagattaagcatggtgttttctaagcaagctttgaaaggggccttccatacttacttaattgaatattctgggatattgaaaattattcagatacttgacaattatttttggttacctactccgcaaactacaaagttttaaggactcaacaataagttaatgagacacagtgtttgctttcatggagcttacagtctggaggggacaaaggcttaaacaatactcatataattatatatgtgatcagtacaatgaaggagctcagtggggtaaataagcaggaacctgaacttgatctgttccggagggccacagaaggcttccttgaggccttgagaaagtgatttgcatctgagttctgaaggattgtaagaggtaactagggaaaaagttgacaggaagaggaaggggatccagacaagaaacatttgcaaagatcttgaggcataaatgagcttgagacatctggagaaactgaggaaaagtgagagagtaggcagggcctggagccgcagagccattgctaaccatcctgtgtgagatatcccccattctgtagctttattctcataaccctgctcaattttctttataacacttctcacagatttatatacgtgtttgtttttgttatctgtctctcccaccagaccacagctccatgagagcaaggtctttgcttaccaatatatcactagcacttaaaactatgcctggtacacagtaggttcttaatatgtgttgaatatagccatcaaattgatattggatataattcaatctgataagatattttgagatattaaagagtttttaacttgataccataaaaaaaaaaaaaaaaa

SEQ ID NO: 13 - DNA, 551 bp, Homo sapiens
gagaggggtatacacagggaggccaggcagcctggagttagtcgaccgttgcgagacgttgagctgcggcagatgagtccaaagccgagagcctcgggacctccggccaaggccaaggagacaggaaagaggaagtcctcctctcagccgagccccagtggcccgaagaagaagactaccaaggtggccgagaagggagaagcagttcgtggagggagacgcgggaagaaaggggctgcgacaaagatggcggccgtgacggcacctgaggcggagagcgggccagcggcacccggccccagcgaccagcccagccaggagctccctcagcacgagctgccgccggaggagccagtgagcgaggggacccagcacgaccccctgagtcaggagagcgagctggaggaaccactgagtaaggggcgcccatctactcccctatctccctgagcagcaactaagtttaggcccagctgccagacctcagagatctcaccagcagggtgcttcccatgttgatgacaataaaatgaatgtgttgcaaaccgaaa

(19) United States
(12) Patent Application Publication
(10) Pub. No.: US 2019/0284531 A1
(43) Pub. Date: Sep. 19, 2019

(54) DETECTION OF T CELL EXHAUSTION OR LACK OF T CELL COSTIMULATION AND USES THEREOF

(71) Applicant: Cambridge Enterprise Limited, Cambridge (GB)

(72) Inventors: Kenneth G.C. Smith, Cambridge (GB); Paul A. Lyons, Cambridge (GB); Eoin F. McKinney, Cambridge (GB)

(21) Appl. No.: 16/422,377

(22) Filed: May 24, 2019

Related U.S. Application Data
(63) Continuation of application No. 15/813,947, filed on Nov. 15, 2017, now abandoned.
Application No. 15/813,947 is a continuation-in-part of application No. PCT/GB2016/051385, filed on May 13, 2016.

(30) Foreign Application Priority Data
May 15, 2015 (GB) 1508419.7

(51) Int. Cl. C12N 5/0783; C12Q 1/6883; C12Q 1/6886; C12Q 1/6816; C12Q 1/6881

(52) U.S. Cl. CPC C12N 5/0636; C12Q 1/6883; C12Q 1/6886; C12Q 2600/112; C12Q 1/6881; C12Q 2600/118; C12Q 2600/158; C12Q 1/6816

(57) ABSTRACT

The application relates to methods of assessing whether an individual has an exhausted CD8⁺ T cell or lack of CD4⁺ T cell costimulation phenotype, and the use of such methods in determining an individual's risk of autoimmune disease progression, progression of a chronic infection, not responding to a treatment for a chronic infection, not mounting an effective immune response to vaccination, infection-associated immunopathology, transplant rejection, or cancer progression. The application also relates to in vitro methods for assessing whether CD8⁺ and CD4⁺ T cells in a sample have an exhausted CD8⁺ T cell or lack of CD4⁺ T cell costimulation phenotype, and for identifying a substance capable of inducing an exhausted CD8⁺ T cell or lack of CD4⁺ T cell costimulation phenotype in an individual, as well as a kit for assessing whether an individual has an exhausted CD8⁺ T cell or lack of CD4⁺ T cell costimulation phenotype or whether an exhausted CD8⁺ T cell or lack of CD4⁺ T cell costimulation phenotype is present in a sample of CD8⁺ and CD4⁺ T cells.

### CROSS-REFERENCE TO RELATED APPLICATIONS

This application is a continuation of, and therefore claims priority from, U.S. patent application Ser. No. 15/813,947 entitled Detection of T Cell Exhaustion or Lack of T Cell Costimulation and Uses Thereof filed Nov. 14, 2017, which claims priority to International Patent Application No. PCT/GB2016/051385, filed on May 13, 2016, designating the United States of America, which derives priority from GB 1508419.7, filed on May 15, 2015. Each of the above-referenced applications is incorporated by reference herein in its entirety.

### FIELD OF THE INVENTION

The present invention relates to methods of assessing whether an individual has an exhausted CD8⁺ T cell or lack of CD4⁺ T cell costimulation phenotype, and the use of such methods in determining an individual's risk of autoimmune disease progression, progression of a chronic infection, not responding to a treatment for a chronic infection, not mounting an effective immune response to vaccination, infection-associated immunopathology, transplant rejection, or cancer progression. Such methods may also be used to guide treatment of autoimmune diseases, chronic infections, infection-associated immunopathology, transplant patients, and cancer. The present invention also relates to in vitro methods for assessing whether CD8⁺ and CD4⁺ T cells in a sample have an exhausted CD8⁺ T cell or lack of CD4⁺ T cell costimulation phenotype, and for identifying a substance capable of inducing an exhausted CD8⁺ T cell or lack of CD4⁺ T cell costimulation phenotype in an individual, as well as a kit for assessing whether an individual has an exhausted CD8⁺ T cell or lack of CD4⁺ T cell costimulation phenotype or whether an exhausted CD8⁺ T cell or lack of CD4⁺ T cell costimulation phenotype is present in a sample of CD8⁺ and CD4⁺ T cells.

### BACKGROUND TO THE INVENTION

Following transient exposure to antigen, such as during acute viral infection, antigen-specific T cells undergo rapid proliferation followed by a more prolonged contraction. Following this, the persistent population of memory cells that remain confer protective immunity, characterised by higher proliferative potential and a reduced activation threshold (E. J. Wherry, R. Ahmed, Journal of virology 78, 5535-5545, 2004). However, where antigen persists or accessory costimulatory signals are limiting, such as during chronic viral infection, CD8 T cells develop a hierarchical loss of function in a process termed “exhaustion” (A. J. Zajac et al., The Journal of experimental medicine 188, 2205-2213, 1998). In models of acute antigen exposure, the primary CD8 response is equivalent in the absence of accessory CD4 costimulation, although long-term memory responses rely upon it (D. J. Shedlock, H. Shen, Science 300, 337-339, 2003; J. C. Sun, M. J. Bevan, Science 300, 339342, 2003). In chronic antigen exposure, CD8 responses - and memory responses in particular—are exquisitely dependent on the provision of CD4 help (E. E. West et al., Immunity 35, 285-298, 2011). Consequently, in both mice and in humans, the presence of

CD4 help results in enhanced viral clearance and resolution of chronic infection while also promoting robust memory responses in acute infection (R. D. Aubert et al., J Exp Med 194, 1395-1406, 2001).

The dysfunctional state of exhaustion was originally identified in a chronic form of murine LCMV infection as cells showing reduced cytokine production (A. J. Zajac et al., The Journal of experimental medicine 188, 2205-2213, 1998) with hierarchical loss of IL2 production and cytolytic killing followed by loss of TNF production and, finally, deletion of antigen-specific cells (D. Moskophidis et al., Nature 362, 758-761, 1993). More recently it has been shown that this progressive dysfunction is accompanied by profound changes in gene expression, distinct from those seen in effector or anergic cells (E. J. Wherry et al., Immunity 27, 670-684, 2007; I. A. Parish et al., Blood 113, 4575-4585, 2009). This transcriptional ‘signature’ of CD8 exhaustion is both characterised and driven by non-redundant patterns of inhibitory receptor coexpression (S. D. Blackburn et al., Nature immunology 10, 29-37, 2009) that can serve as both biomarkers of viral progression (C. L. Day et al., Nature 443, 350-354, 2006) and targets for therapy, helping to restore viral control (D. L. Barber et al., Nature 439, 682-687, 2006).

Exhausted T cell responses have likewise been implicated in loss of immunological control of cancers (H. W. Virgin et al., Cell 138, 30-50, 2009). A signature of CD8 T cell exhaustion has been described within the tumour microenvironment (Baitsch et al., JCI, 121(6):2350, 2011) and exhaustion-associated inhibitory receptor blockade (known as ‘checkpoint blockade) has shown promise in clinical trials (Topalian et al., N Engl J Med 366, 2443-245; Pardoll, Nat Rev Cancer 12:252, 2012; Phan et al. PNAS100:8372-7, 2003).

It has also been speculated that a similar phenotype of exhaustion may be seen in the context of organ or tissue transplantation (Valujskikh A, Li XC., Curr Opin Organ Transplant. 2012, 17:15-19), due primarily to the known role of exhaustion-associated inhibitory receptors in the process of immunoregulation (Thorp et al. Curr Opin Org Transplant 2015, 20(1):37-42). While small, studies in murine transplant models have argued that T cell exhaustion may limit acute rejection (Steger et al., Transplantation. 2008, 85:1339-1347) or chronic rejection (Sarraj et al., Proc Natl Acad Sci U S A. 2014;111:12145-12150).

The process of T cell exhaustion has also been implicated in the success of vaccination strategies. The goal of vaccination is to produce long-lasting, antigen-specific immunity that will protect the subject from infection, to eradicate an existing infection or, in the case of cancer vaccines, to eradicate a tumour. During persistent infection, blockade of exhaustion-associated pathways allows an otherwise ineffective vaccine to successfully enhance viral clearance (Brooks et al. JEM 2008, 205(3):533-41).

In chronic viral infection higher levels of CD4 help are associated with diminished CD8 T cell exhaustion (Thimme et al., J Exp Med 194, 1395-1406, 2001; Aubert et al., Proc Natl Aced Sci U S A 108, 21182-21187), while during vaccination, costimulation promoted by adjuvants, can encourage long-lasting immune responses (Reed et al., Nat Med 2013; 19: 1597-1608). The potential for adjuvants to encourage such a protective response has been known for almost a century (Glenny et al., J Pathol Bacteriol 1926; 29: 38-45). However, it remains unclear which adjuvants provide optimal protection (Reed et al., Nat Med 2013; 19: 1597-1608). Reversing exhaustion-associated inhibitory pathways through targeted costimulation or inhibitory receptor blockade has shown some promise in maintaining effective immune responses (Wang et al., Cell Mol Immunol 2014; Vezys et al., J Immunol 2011; 187: 1634-1642).

A role for individual exhaustion-associated inhibitory receptors in autoimmunity has also been demonstrated—both through GWAS association results (Gateva et al., Nat Genet 41, 1228-1233, 2009; Lee et al Lupus 18, 9-15, 2009; Prokunina et al., Nat Genet 32, 666-669, 2002) and functional studies in mice, showing an increasingly severe phenotype in their absence (Keir et al., Annu Rev Immunol 26, 677-704, 2008; Rangachari, et al., Bat3 promotes T cell responses and autoimmunity by repressing Tim-3-mediated cell death and exhaustion. Nat Med; Okazaki et al., PD-1 and LAG-3 inhibitory co-receptors act synergistically to prevent autoimmunity in mice. J Exp Med 208, 395-407).

The underlying process of T cell exhaustion has been implicated in each of the contexts outlined above. As a result there is substantial and increasing interest in either reversing (in cancer, chronic infection or vaccination), preventing (in vaccination) or promoting the process (in autoimmune and infection-associated immunopathology and transplantation) to improve clinical outcomes for patients. Although recent progress has been made in both chronic infection (Day et al. Nature 2006;443:350) and cancer (Herbst et al. Nature 2014;515:563), there currently exists no method of measuring T cell exhaustion that is sufficiently robust or validated to allow prediction of outcome, response to therapy or to allow targeted therapy in patients with infection, cancer, autoinfection-associated immunopathology or who are receiving a vaccination or a transplant. K(lysine) acetyltransferase 2B (KAT2B), also known as P300/CBP-associated factor (PCAF), was originally identified as a histone acetyltransferase, which promotes cell-cycle arrest and counteracts the mitogenic influence of the adenoviral E1A oncoprotein (Yang, Xiang-Jiao et al. Nature 1996;382:319-24). In addition, KAT2B has been shown to mediate an anti-apoptotic effect under conditions of metabolic stress (Xenaki, G. et al. Oncogene 2008;27:5785-96), increasing cellular resistance to cytotoxic compounds when overexpressed (Hirano et al. Mol Cancer Research 2010;8(6):864-72). KAT2B contains both bromodomain and histone acetyltransferase regions, which confer the capacity to both ‘read’ and ‘write’ epigenetic marks and act as a transcriptional co-activator. KAT2B has also been described as playing a role in hepatic gluconeogenesis, glucose metabolism and glucose homeostasis and has been proposed as target for diabetes treatment (Annicotte J.-S., 2014 Seminar Of The Lausanne Integrative Metabolism And Nutrition Alliance; Annicotte et al., 2013, Diabetes and Metabolism, 39(S1):Al2; Sun et al., 2014 Cell Rep. 24;9(6):2250-62; Ravnskjaer et al. 2013 J Clin Invest. 123(10): 4318-4328; W02014/037362; W02013/148114). KAT2B has also been proposed as a marker of autoimmune disease progression (W02010/084312) but has not been linked with an exhausted CD8⁺ T cell or lack of CD4⁺ T cell costimulation phenotype.

### SUMMARY OF THE INVENTION

The present inventors have identified gene expression signatures that can be used to identify an exhausted CD8⁺ T cell or lack of CD4⁺ T cell costimulation phenotype, or a non-exhausted CD8⁺ T cell or CD4⁺ T cell costimulation phenotype. Identification of these phenotypes in individuals is expected to be useful in assessing an individual's risk of: autoimmune disease progression, progression of a chronic infection, not responding to a treatment for a chronic infection, not mounting an effective immune response to a vaccine, infection-associated immunopathology, transplant rejection, or cancer progression, as well as guiding therapy in autoimmune diseases, chronic infection, vaccination, infection-associated immunopathologies, transplantation, and cancer.

An aspect of the invention provides a method of assessing whether an individual has an exhausted CD8⁺ T cell or lack of CD4⁺ T cell costimulation phenotype. The method comprises establishing, by determining the expression level of two more genes selected from the group consisting of:
K(lysine) acetyltransferase 2B gene (KAT2B);
calcium/calmodulin-dependent serine protein kinase 3 gene (CASK);
   ATP-binding cassette sub-family D member 2 gene (ABCD2);
      disks large homolog 1 gene (DLG1);
   synovial sarcoma translocation, chromosome 18 gene (SS18);
      Retinoblastoma-like protein 2 gene (RBL2);
      RAS oncogene family-like 1 gene (RAB7L1);
   methylenetetrahydrofolate dehydrogenase 1 gene (MTHFDI);
      keratoca gene (KERA);
      B cell-specific Moloney murine leukemia virus integration site 1 gene (BMI1);
      conserved oligomeric Golgi complex subunit 5 gene (COG5);
   cAMP-specific 3′,5′-cyclic phosphodiesterase 4D gene (PDE4D); and
      variable charge, Y-linked gene (VCY);
in a sample obtained from the individual, whether said subject has said phenotype,
wherein said phenotype is characterised by upregulated expression of genes KAT2B, CASK, ABCD2, DLG1, SS18, RBL2, RAB7L1, MTHFDI, BMI1, COG5, and PDE4D, and downregulated expression of genes KERA and VCY, relative to the level of expression of these genes in an individual who does not have said phenotype.

Other aspects of the invention provide methods of assessing whether an individual is at high risk or low risk of autoimmune disease progression, progression of a chronic infection, not responding to a treatment for a chronic infection, not mounting an effective immune response to vaccination, infection-associated immunopathology, transplant rejection, or cancer progression, as set out in the claims.

Yet other aspects of the invention provide methods of treating, or selecting for treatment, individuals who have been identified using a method of the invention as being at high risk or low risk of autoimmune disease progression, progression of a chronic infection, not responding to a treatment for a chronic infection, infection-associated immunopathology, cancer progression, not mounting an effective immune response to vaccination, or transplant rejection, employing a method as set out above, as set out in the claims. Treatment may comprise inducing an exhausted CD8⁺ T cell or lack of CD4⁺ T cell costimulation phenotype, or inducing a non-exhausted CD8⁺ T cell or CD4⁺ T cell costimulation phenotype, in the individual, as appropriate, to decrease the risk of autoimmune disease or cancer progression, progression of a chronic infection, not responding to a treatment for a chronic infection, infection-associated immunopathology, transplant rejection, or not mounting an effective immune response to vaccination.

An in vitro method for assessing whether CD8⁺ and CD4⁺ T cells in a sample have an exhausted CD8⁺ T cell or lack of CD4⁺ T cell costimulation phenotype, and an in vitro method for identifying a substance capable of inducing an exhausted CD8⁺ T cell or lack of CD4⁺ T cell costimulation phenotype in an individual, as set out in the claims, are similarly provided as aspects of the invention, as is a method of preparing CD8+T cells with an exhausted or non-exhausted CD8⁺ T cell phenotype.

A kit for assessing whether an individual has an exhausted CD8⁺ T cell or lack of CD4⁺ T cell costimulation phenotype, or whether an exhausted CD8⁺ T cell or lack of CD4⁺ T cell costimulation phenotype is present in a sample of CD8⁺ and CD4⁺ T cells forms a further aspect of the invention.

The following numbered paragraphs set out additional embodiments of the invention.

Paragraph 1. A method of assessing whether an individual has an exhausted CD8⁺ T cell or lack of CD4⁺ T cell costimulation phenotype, the method comprising establishing, by determining the expression level of two more genes selected from the group consisting of: K(lysine) acetyltransferase 2B gene (KAT2B);
calcium/calmodulin-dependent serine protein kinase 3 gene (CASK);
   ATP-binding cassette sub-family D member 2 gene (ABCD2);
   disks large homolog 1 gene (DLG1);
synovial sarcoma translocation, chromosome 18 gene (SS18);
   Retinoblastoma-like protein 2 gene (RBL2);
   RAS oncogene family-like 1 gene (RAB7L1);
   methylenetetrahydrofolate dehydrogenase 1 gene (MTHFDI);
   keratocan gene (KERA);
   B cell-specific Moloney murine leukemia virus integration site 1 gene (BMI1);
conserved oligomeric Golgi complex subunit 5 gene (COG5);
cAMP-specific 3′,5′-cyclic phosphodiesterase 4D gene (PDE4D); and
   variable charge, Y-linked gene (VCY);
in a sample obtained from the individual, whether said subject has said phenotype,
wherein said phenotype is characterised by downregulated expression of genes KAT2B, CASK, ABCD2, DLG1, SS18, RBL2, RAB7L1, MTHFDI, BMI1, COG5, and PDE4D, and upregulated expression of genes KERA and VCY, relative to the level of expression of these genes in an individual who does not have said phenotype.

Paragraph 2. A method of assessing whether an individual is at high risk or low risk of autoimmune disease progression, the method comprising:
(i) determining whether the individual has an exhausted CD8⁺ T cell or lack of CD4⁺ T cell costimulation phenotype using the method of paragraph 1,

wherein the presence of said phenotype indicates that the individual is at low risk of autoimmune disease progression, and
wherein the absence of said phenotype indicates that the individual is at high risk of autoimmune disease progression.

Paragraph 3. A method of assessing whether an individual is at low risk or high risk of autoimmune disease progression, by determining whether the individual has or does not have an exhausted CD8⁺ T cell or lack of CD4⁺ T cell costimulation phenotype, the method comprising:
(i) providing a PBMC sample obtained from the individual;
(ii) extracting mRNA from the PBMC sample;
(iii) performing reverse transcription quantitative PCR (RT-qPCR) to convert the mRNA into cDNA and determine the expression level of two more genes selected from the group consisting of: KAT2B, CASK, ABCD2, DLG1, SS18, RBL2, RAB7L1, MTHFDI, KERA, BMI1, COG5, PDE4D, and VCY,

wherein said phenotype is characterised by downregulated expression of genes KAT2B, CASK, ABCD2, DLG1, SS18, RBL2, RAB7L1, MTHFDI, BMI1, COG5, and PDE4D, and upregulated expression of genes KERA and VCY, relative to the level of expression of these genes in an individual who does not have said phenotype, and

wherein the presence of said phenotype indicates that the individual is at low risk of autoimmune disease progression, and

wherein the absence of said phenotype indicates that the individual is at high risk of autoimmune disease progression.

Paragraph 4. The method according to any one of Paragraphs 2 to 3, wherein the autoimmune disease is not rheumatoid arthritis (RA) or inflammatory bowel disease (IBD).

Paragraph 5. The method according to Paragraph 4, wherein the autoimmune disease is selected from the group consisting of: ANCA-associated vasculitis (AAV), systemic lupus erythematosus (SLE), type 1 diabetes, and idiopathic pulmonary fibrosis (IPF).

Paragraph 6. The method according to any one of Paragraphs 2 to 5, wherein the autoimmune disease is AAV or SLE, and
wherein an individual who is at low risk of autoimmune disease progression is at low risk of relapses or flares of the disease, and
wherein an individual who is at high risk of autoimmune disease progression is at high risk of relapses or flares of the disease.

Paragraph 7. The method according to Paragraph 2 to 5, wherein the autoimmune disease is type 1 diabetes, and
wherein an individual who is at low risk of autoimmune disease progression is at low risk of progressing to type 1 diabetes, and

wherein an individual who is at high risk of autoimmune disease progression is at high risk of progressing to type 1 diabetes.

Paragraph 8. The method according to any one of Paragraphs 2-5, wherein the autoimmune disease is IPF, and

wherein an individual who is at low risk of autoimmune disease progression is at low risk of ongoing reduction of lung function, and
wherein an individual who is at high risk of autoimmune disease progression is at high risk of ongoing reduction of lung function.

Paragraph 9. An autoimmune disease progression risk assessment system for use in a method as defined in any one of Paragraphs 2 to 8, the system comprising a tool or tools for determining expression of two more genes selected from the group consisting of:
KAT2B, CASK, ABCD2, DLGI, SS18, RBL2, RAB7L1, MTHFDI, KERA, BMII , COG5, PDE4D, and VCY;

and a computer programmed to compute an autoimmune disease progression risk score from the gene expression data of the subject.

Paragraph 10. The method according to any one of Paragraphs 2 to 8, further comprising: (ii)
selecting an individual identified as one who is at high risk or low risk of autoimmune disease progression in step (i) for treatment for the autoimmune disease.

Paragraph 11. The method according to any one of Paragraphs 2 to 8, further comprising: (ii)
subjecting an individual identified as one who is at high risk or low risk of autoimmune disease progression in step (i) to treatment for the autoimmune disease.

Paragraph 12. A method for treating an autoimmune disease in an individual, the method comprising:
(i) identifying the individual as one who is at high risk or low risk of autoimmune disease progression using a method according to any one of Paragraphs 2 to 8, and
(ii) subjecting the individual to treatment for the autoimmune disease.

Paragraph 13. A method for treating an autoimmune disease in an individual, the method comprising:

(i) requesting a test providing the results of an analysis to determine the expression level of two more genes selected from the group consisting of: KAT2B, CASK, ABCD2, DLG1, SS18, RBL2, RAB7L1, MTHFDI, KERA, BMI1, COG5, PDE4D, and VCY,
in a sample obtained from the individual,
wherein downregulated expression of genes KAT2B, CASK, ABCD2, DLG1, SS18, RBL2, RAB7L1, MTHFDI, BMI1, COG5, and PDE4D, and upregulated expression of genes KERA and VCY, relative to the level of expression of these genes in an individual who does not have an exhausted CD8⁺ T cell or lack of CD4⁺ T cell costimulation phenotype indicates that the individual has said phenotype, and
wherein upregulated expression of genes KAT2B, CASK, ABCD2, DLG1, SS18, RBL2, RAB7L1, MTHFDI, BMI1, COG5, and PDE4D, and downregulated expression of genes KERA and VCY, relative to the level of expression of these genes in an individual who has an exhausted CD8⁺ T cell or lack of CD4⁺ T cell costimulation phenotype indicates that the individual does not have said phenotype, and

wherein the presence of said phenotype indicates that the individual is at low risk of autoimmune disease progression, and

wherein the absence of said phenotype indicates that the individual is at high risk of autoimmune disease progression,
(ii) treating the individual for the autoimmune disease.

Paragraph 14. The method according to any one of Paragraphs 10 to 13, wherein the treatment comprises inducing an exhausted CD8⁺ T cell or lack of CD4⁺ T cell costimulation phenotype in the individual.

Paragraph 15. The method according to Paragraph 14, wherein an exhausted CD8⁺ T cell or lack of CD4⁺ T cell costimulation phenotype is induced in the individual by administering a therapeutically effective amount of a programmed cell death protein 1 (PD-1) ligand.

Paragraph 16. The method according to Paragraph 15, wherein the PD-1 ligand is programmed death-ligand 1 (PDL-1).

Paragraph 17. The method according to any one of Paragraphs 10 to 13, wherein the autoimmune disease is AAV or SLE and wherein individual has been identified as one who is at high risk of autoimmune disease progression,
wherein the treatment comprises treatment with a more frequent or more intense disease treatment regimen, or with a disease regimen not normally administered during the maintenance phase of the autoimmune disease.

Paragraph 18. The method according to any one of Paragraphs 10 to 13, wherein the autoimmune disease is AAV or SLE and wherein individual has been identified as one who is at low risk of autoimmune disease progression,
wherein the treatment comprises treatment with a less frequent or less intense disease treatment regimen, or with a disease regimen not normally administered during the maintenance phase of the autoimmune disease.

Paragraph 19. The method according to any one of Paragraphs 10 to 13, wherein individual has been identified as one who is at high risk of progressing to type 1 diabetes,
wherein the treatment comprises a prophylactic treatment for type 1 diabetes.

Paragraph 20. The method according to any one of Paragraphs 10 to 13, wherein the autoimmune disease is IPF and wherein individual has been identified as one who is at high risk of autoimmune disease progression,

wherein the treatment comprises treatment with nintedanib, pirfenidone, a phosphodiesterase inhibitor, or immunosuppressive therapy.

Paragraph 21. A programmed cell death protein 1 (PD-1) ligand for use in a method of treating an autoimmune disease in an individual, the method comprising

(i) determining whether the individual is at high risk of autoimmune disease progression using a method according to any one of Paragraphs 2 to 8, and
(ii) administering therapeutically effective amount of a PD-1 ligand to the individual if the individual is at high risk of autoimmune disease progression to induce an exhausted CD8⁺ T cell or lack of CD4⁺ T cell costimulation phenotype in the individual.

Paragraph 22. A PD-1 ligand for use according to Paragraph 21, wherein the PD-1 ligand is programmed death-ligand 1 (PDL-1).

Paragraph 23. An in vitro method of assessing whether CD8⁺ and CD4⁺ T cells in a sample have an exhausted CD8⁺ T cell or lack of CD4⁺ T cell costimulation phenotype, the method comprising establishing, by determining the expression level of two more genes selected from the group consisting of: KAT2B, CASK, ABCD2, DLG1, SS18, RBL2, RAB7L1, MTHFDI, KERA, BMI1, COG5, PDE4D, and VCY, whether said phenotype is present, wherein said phenotype is characterised by downregulated expression of genes KAT2B, CASK, ABCD2, DLG1, SS18, RBL2, RAB7L1, MTHFDI, BMI1, COG5, and PDE4D, and
upregulated expression of genes KERA and VCY relative to the level of expression of these genes in a sample of CD8⁺ and CD4⁺ T cells which do not have said phenotype.

Paragraph 24. The method according to any one of Paragraphs 2 to 5, 7, 10 to 16, or 19, wherein the autoimmune disease is type 1 diabetes, and wherein the individual is genetically predisposed to type 1 diabetes.

Paragraph 25. The method according to Paragraph 24, wherein the individual is a child.

Paragraph 26. The method according to any one of Paragraphs 24 to 25, wherein the individual has a HLA genotype which is associated with a high risk of type 1 diabetes.

Paragraph 27. The method according to any one of Paragraphs 24 to 26, wherein the individual has a mother, father and/or sibling with type 1 diabetes.

Paragraph 28. The method according to any one of Paragraphs 24 to 27, wherein the individual does not have autoantibodies associated with type 1 diabetes.

Paragraph 29. A method of assessing whether an individual with a chronic infection is at high risk or low risk of progression of said chronic infection, the method comprising:
(i) determining whether the individual has an exhausted CD8⁺ T cell or lack of CD4⁺ T cell costimulation phenotype using the method of Paragraph 1,
wherein the presence of said phenotype indicates that the individual is at high risk of progression of said chronic infection, and

wherein the absence of said phenotype indicates that the individual is at low risk of progression of said chronic infection.

Paragraph 30. A method of assessing whether an individual with a chronic infection is at high risk or low risk of progression of said chronic infection, by determining whether the individual has or does not have an exhausted CD8⁺ T cell or lack of CD4⁺ T cell costimulation phenotype, the method comprising:
(i) providing a PBMC sample obtained from the individual;
(ii) extracting mRNA from the PBMC sample;

(iii) performing reverse transcription quantitative PCR (RT-qPCR) to convert the mRNA into cDNA and determine the expression level of two more genes selected from the group consisting of: KAT2B, CASK, ABCD2, DLG1, SS18, RBL2, RAB7L1, MTHFDI, KERA, BMI1, COG5, PDE4D, and VCY,
wherein said phenotype is characterised by downregulated expression of genes KAT2B, CASK, ABCD2, DLG1, SS18, RBL2, RAB7L1, MTHFDI, BMI1, COG5, and PDE4D, and upregulated expression of genes KERA and VCY, relative to the level of expression of these genes in an individual who does not have said phenotype, and
wherein the presence of said phenotype indicates that the individual is at high risk of progression of said chronic infection, and

wherein the absence of said phenotype indicates that the individual is at low risk of progression of said chronic infection.

Paragraph 31. A chronic infection progression risk assessment system to determine the risk of an individual with a chronic infection, for use in a method as defined in any one of Paragraphs 29 to 30, the system comprising a tool or tools for determining expression of two more genes selected from the group consisting of: KAT2B, CASK, ABCD2, DLG1, SS18, RBL2, RAB7L1, MTHFDI, KERA, BMI1, COG5, PDE4D, and VCY;
and a computer programmed to compute a risk score of the risk of the individual not responding to the treatment from the gene expression data of the subject.

Paragraph 32. The method according to any one of Paragraphs 29 to 30, further comprising: (ii) selecting an individual identified as one who is at high risk of progression of the chronic infection in step (i) for treatment for said chronic infection.

Paragraph 33. The method according to any one of Paragraphs 29 to 30, further comprising:
(ii) subjecting the individual to treatment for the chronic infection if the individual has been identified as one who is at high risk of progression of the chronic infection in step (i).

Paragraph 34. A method for treating a chronic infection in an individual, the method comprising:
(i) identifying the individual as one who is at high risk of progression of the chronic infection using a method according to any one of Paragraphs 29 to 30, and
(ii) subjecting the individual to c treatment for said chronic infection.

Paragraph 35. A method for treating a chronic infection in an individual, the method comprising:

(i) requesting a test providing the results of an analysis to determine the expression level of two more genes selected from the group consisting of: KAT2B, CASK, ABCD2, DLG1, SS18, RBL2, RAB7L1, MTHFDI, KERA, BMI1, COG5, PDE4D, and VCY, in a sample obtained from the individual,

wherein downregulated expression of genes KAT2B, CASK, ABCD2, DLG1, SS18, RBL2, RAB7L1, MTHFDI, BMI1, COG5, and PDE4D, and upregulated expression of genes KERA and VCY, relative to the level of expression of these genes in an individual who does not have an exhausted CD8⁺ T cell or lack of CD4⁺ T cell costimulation phenotype indicates that the individual has said phenotype, and
(ii) subjecting the individual to treatment for said chronic infection if the individual has said phenotype.

Paragraph 36. The method according to any one of Paragraphs 29 to 30, further comprising:

(ii) selecting an individual identified as one who is at high risk of progression of the chronic infection in step (i) for treatment,

wherein the treatment comprises inducing a non-exhausted CD8⁺ T cell or CD4⁺ T cell costimulation phenotype in the individual.

Paragraph 37. The method according to any one of Paragraphs 29 to 30, further comprising:
(ii) subjecting the individual to treatment if the individual has been identified as one who is at high risk of progression of the chronic infection in step (i),
wherein the treatment comprises inducing a non-exhausted CD8⁺ T cell or CD4⁺ T cell costimulation phenotype in the individual.

Paragraph 38. A method for treating a chronic infection in an individual, the method comprising:
(i) identifying the individual as one who is at high risk of progression of the chronic infection using a method according to any one of Paragraphs 29 to 30, and
(ii) subjecting the individual to treatment if the individual has been identified as one who is at high risk of progression of the chronic infection in step (i),

wherein the treatment comprises inducing a non-exhausted CD8⁺ T cell or CD4⁺ T cell costimulation phenotype in the individual.

Paragraph 39. A method for treating a chronic infection in an individual, the method comprising:
(i) requesting a test providing the results of an analysis to determine the expression level of two more genes selected from the group consisting of: KAT2B, CASK, ABCD2, DLG1, SS18, RBL2, RAB7L1, MTHFDI, KERA, BMI1, COG5, PDE4D, and VCY,
in a sample obtained from the individual,

downregulated expression of genes KAT2B, CASK, ABCD2, DLG1, SS18, RBL2, RAB7L1, MTHFDI, BMI1, COG5, and PDE4D, and upregulated expression of genes KERA and VCY, relative to the level of expression of these genes in an individual who does not have an exhausted CD8⁺ T cell or lack of CD4⁺ T cell costimulation phenotype indicates that the individual has said phenotype, and
(ii) subjecting the individual to a treatment for the chronic infection with the treatment

if the individual has said phenotype,

wherein the treatment comprises inducing a non-exhausted CD8⁺ T cell or CD4⁺ T cell costimulation phenotype in the individual.

Paragraph 40. The method according to any one of Paragraphs 36 to 39, wherein a non-exhausted CD8⁺ T cell or CD4⁺ T cell costimulation phenotype is induced in the individual by administering a therapeutically effective amount of an inhibitor of programmed cell death protein 1 (PD-1).

Paragraph 41. A PD-1 inhibitor for use in a method of treating a chronic infection in an individual, the method comprising
(i) determining whether the individual is at high risk of progression of the chronic infectionusing a method according to any one of Paragraphs 29 to 30, and
(ii) administering therapeutically effective amount of a PD-1 ligand to the individual if the individual is at high risk of progression of the chronic infection to induce a non-exhausted CD8⁺ T cell or CD4⁺ T cell costimulation phenotype in the individual.

Paragraph 42. The method according to any one of Paragraphs 29 to 30, 32 to 40, the risk assessment system according to Paragraph 31, or the PDI inhibitor for use according to Paragraph 41, wherein the chronic infection is a chronic viral infection, a chronic bacterial infection or a chronic parasitic infection.

Paragraph 43. A method of assessing whether an individual is at high risk or low risk of not mounting an effective immune response to a vaccine against a disease, the individual having received the vaccination, wherein the method comprises:
(i) determining whether the individual has an exhausted CD8⁺ T cell or lack of CD4⁺ T cell costimulation phenotype using the method of Paragraph 1,
wherein the presence of said phenotype indicates that the individual at high risk of not mounting effective immune response to the vaccination, and

wherein the absence of said phenotype indicates that the individual is at low risk of not mounting effective immune response to the vaccination.

Paragraph 44. A method of assessing whether an individual at high risk or low risk of not mounting an effective immune response to a vaccine against a disease, the individual having received the vaccination, by determining whether the individual has or does not have an exhausted CD8⁺ T cell or lack of CD4⁺ T cell costimulation phenotype, the method comprising:
(i) providing a PBMC sample obtained from the individual;
(ii) extracting mRNA from the PBMC sample;

(iii) performing reverse transcription quantitative PCR (RT-qPCR) to convert the mRNA into cDNA and determine the expression level of two more genes selected from the group consisting of: KAT2B, CASK, ABCD2, DLG1, SS18, RBL2, RAB7L1, MTHFDI, KERA, BMI1, COG5, PDE4D, and VCY,
wherein said phenotype is characterised by downregulated expression of genes KAT2B, CASK, ABCD2, DLG1, SS18, RBL2, RAB7L1, MTHFDI, BMI1, COG5, and PDE4D, and upregulated expression of genes KERA and VCY, relative to the level of expression of these genes in an individual who does not have said phenotype, and
wherein the presence of said phenotype indicates that the individual at high risk of not mounting effective immune response to the vaccination, and

wherein the absence of said phenotype indicates that the individual is at low risk of not mounting effective immune response to the vaccination.

Paragraph 45. A vaccination non-response risk assessment system for use in a method as defined in any one of Paragraphs 43 to 44, the system comprising a tool or tools for determining expression of two more genes selected from the group consisting of: KAT2B, CASK, ABCD2, DLG1, SS18, RBL2, RAB7L1, MTHFDI, KERA, BMI1, COG5, PDE4D, and VCY;
and a computer programmed to compute an vaccination non-response risk score from the gene expression data of the subject.

Paragraph 46. The method according to any one of Paragraphs 43 to 44, further comprising:
(ii) selecting an individual identified as one who is at high risk of not mounting an effective immune response to a vaccine in step (i) for vaccination with a further dose of the same vaccine, or with a different vaccine against the same disease.

Paragraph 47. The method according to any one of Paragraphs 43 to 44, further comprising:
(ii) subjecting the individual to vaccination with a further dose of the same vaccine, or with a different vaccine against the same disease, if the individual has been identified as one who is at high risk of not mounting an effective immune response to a vaccine in step (i).

Paragraph 48. A method for vaccinating an individual, the method comprising:
(i) identifying the individual as one who is at high risk of not mounting an effective immune response to a vaccine using a method according to any one of Paragraphs 43 to 44, and
(ii) subjecting the individual to vaccination with a further dose of the same vaccine, or with a different vaccine against the same disease.

Paragraph 49. A method for vaccinating an individual, the method comprising:
(i) vaccinating the individual;
(ii) requesting a test providing the results of an analysis to determine the expression level of two more genes selected from the group consisting of: KAT2B, CASK, ABCD2, DLG1, SS18, RBL2, RAB7L1, MTHFDI, KERA, BMI1, COG5, PDE4D, and VCY, in a sample obtained from the individual,
wherein downregulated expression of genes KAT2B, CASK, ABCD2, DLG1, SS18, RBL2, RAB7L1, MTHFDI, BMI1, COG5, and PDE4D, and upregulated expression of genes KERA and VCY, relative to the level of expression of these genes in an individual who does not have an exhausted CD8⁺ T cell or lack of CD4⁺ T cell costimulation phenotype indicates that the individual has said phenotype, and
(iii)subjecting the individual to vaccination with a further dose of the same vaccine, or with a different vaccine against the same disease, if the individual has said phenotype. 30

Paragraph 50. The method according to any one of Paragraphs 46 to 49, wherein the individual is subjected to treatment, or selected for subjection to treatment, with a treatment for inducing a non-exhausted CD8⁺ T cell or CD4⁺ T cell costimulation phenotype in the individual prior to vaccination of the individual with a further dose of the vaccine.

Paragraph 51. The method according to Paragraph 50, wherein a non-exhausted CD8⁺ T cell or CD4⁺ T cell costimulation phenotype is induced in the individual by administering a therapeutically effective amount of an inhibitor of programmed cell death protein 1 (PD-1).

Paragraph 52. A PD-1 inhibitor for use in a method of vaccinating an individual, the method comprising
(i) determining whether the individual is at high risk of not mounting an effective immune response to a vaccine using a method according to any one of Paragraphs 43 to 44, and

(ii) administering therapeutically effective amount of a PD-1 ligand to the individual if the individual is at high risk of not mounting an effective immune response to a vaccine to induce a non-exhausted CD8⁺ T cell or CD4⁺ T cell costimulation phenotype in the individual;
(iii) subjecting the individual to vaccination with a further dose of the same vaccine, or with a different vaccine against the same disease.

Paragraph 53. A method of assessing whether an individual is at high risk or low risk of infection-associated immunopathology, the method comprising:
(i) determining whether the individual has an exhausted CD8⁺ T cell or lack of CD4⁺ T cell costimulation phenotype using the method of Paragraph 1,

wherein the presence of said phenotype indicates that the individual is at low risk of infection-associated immunopathology, and
wherein the absence of said phenotype indicates that the individual is at high risk of infection-associated immunopathology.

Paragraph 54. A method of assessing whether an individual is at low risk or high risk of infection-associated immunopathology, by determining whether the individual has or does not have an exhausted CD8⁺ T cell or lack of CD4⁺ T cell costimulation phenotype, the method comprising:
(i) providing a PBMC sample obtained from the individual;
(ii) extracting mRNA from the PBMC sample;
(iii) performing reverse transcription quantitative PCR (RT-qPCR) to convert the mRNA into cDNA and determine the expression level of two more genes selected from the group consisting of: KAT2B, CASK, ABCD2, DLGI, SSI8, RBL2, RAB7LI, MTHFDI, KERA, BMII , COG5, PDE4D, and VCY,

wherein said phenotype is characterised by downregulated expression of genes KAT2B, CASK, ABCD2, DLGI, SSI8, RBL2, RAB7LI, MTHFDI, BMII , COG5, and PDE4D, and upregulated expression of genes KERA and VCY, relative to the level of expression of these genes in an individual who does not have said phenotype, and
wherein the presence of said phenotype indicates that the individual is at low risk of infection-associated immunopathology, and

wherein the absence of said phenotype indicates that the individual is at high risk of infection-associated immunopathology.

Paragraph 55. An infection-associated immunopathology risk assessment system for use in a method as defined in any one of Paragraphs 53 to 54, the system comprising a tool or tools for determining expression of two more genes selected from the group consisting of:
KAT2B, CASK, ABCD2, DLG1, SS18, RBL2, RAB7L1, MTHFD1, KERA, BMI1, COG5, PDE4D, and VCY;
and a computer programmed to compute an infection-associated immunopathology risk score from the gene expression data of the subject.

Paragraph 56. The method according to any one of Paragraphs 53 to 54, further comprising:
(ii) selecting an individual identified as one who is at high risk of infection-associated immunopathology in step (i) for treatment for the infection-associated immunopathology.

Paragraph 57. The method according to any one of Paragraphs 53 to 54, further comprising:
(ii) subjecting an individual identified as one who is at high risk of infection-associated immunopathology in step (i) to treatment for the infection-associated immunopathology.

Paragraph 58. A method for treating an infection-associated immunopathology in an individual, the method comprising:
(i) identifying the individual as one who is at high risk of infection-associated immunopathology using a method according to any one of Paragraph 53 to 54, and
(ii) subjecting the individual to treatment for the infection-associated immunopathology.

Paragraph 59. A method for treating an infection-associated immunopathology in an individual, the method comprising:
(i) requesting a test providing the results of an analysis to determine the expression level of two more genes selected from the group consisting of: KAT2B, CASK, ABCD2, DLG1, SS18, RBL2, RAB7L1, MTHFD1, KERA, BMI1, COG5, PDE4D, and VCY, in a sample obtained from the individual,
wherein upregulated expression of genes KAT2B, CASK, ABCD2, DLG1, SS18, RBL2, RAB7L1, MTHFDI, BMII , COG5, and PDE4D, and downregulated expression of genes KERA and VCY, relative to the level of expression of these genes in an individual who has an exhausted CD8⁺ T cell or lack of CD4⁺ T cell costimulation phenotype indicates that the individual does not have said phenotype, and

wherein the absence of said phenotype indicates that the individual is at high risk of infection-associated immunopathology,
(ii) treating the individual for the infection-associated immunopathology of the individual does not have said phenotype.

Paragraph 60. The method according to any one of Paragraphs 56 to 59, wherein the treatment comprises inducing an exhausted CD8⁺ T cell or lack of CD4⁺ T cell costimulation phenotype in the individual.

Paragraph 61. The method according to Paragraph 60, wherein an exhausted CD8⁺ T cell or lack of CD4⁺ T cell costimulation phenotype is induced in the individual by administering a therapeutically effective amount of a PD-1 ligand.

Paragraph 62. The method according to Paragraph 61, wherein the PD-1 ligand is PDL-I.

Paragraph 63. A PD-1 ligand for use in a method of treating an infection-associated immunopathology in an individual, the method comprising
(i) determining whether the individual is at high risk of infection-associated immunopathology using a method according to any one of Paragraphs 53 to 54, and (ii) administering therapeutically effective amount of a PD-1 ligand to the individual if the individual is at high risk of infection-associated immunopathology to induce an exhausted CD8⁺ T cell or lack of CD4⁺ T cell costimulation phenotype in the individual.

Paragraph 64. A PD-1 ligand for use according to Paragraph 63, wherein the PD-1 ligand is PDL-I.

Paragraph 65. A method of assessing whether an individual is at high risk or low risk of transplant rejection, the method comprising:
(i) determining whether the individual has an exhausted CD8⁺ T cell or lack of CD4⁺ T cell costimulation phenotype using the method of Paragraph 1,

wherein the presence of said phenotype indicates that the individual is at low risk of transplant rejection, and
wherein the absence of said phenotype indicates that the individual is at high risk of transplant rejection.

Paragraph 66. A method of assessing whether an individual is at high risk or low risk of transplant rejection, by determining whether the individual has or does not have an exhausted CD8⁺ T cell or lack of CD4⁺ T cell costimulation phenotype, the method comprising:
(i) providing a PBMC sample obtained from the individual;
(ii) extracting mRNA from the PBMC sample;

(iii) performing reverse transcription quantitative PCR (RT-qPCR) to convert the mRNA into cDNA and determine the expression level of two more genes selected from the group consisting of: KAT2B, CASK, ABCD2, DLG1, SS18, RBL2, RAB7L1, MTHFDI, KERA, BMI1, COG5, PDE4D, and VCY,
wherein said phenotype is characterised by downregulated expression of genes KAT2B, CASK, ABCD2, DLG1, SS18, RBL2, RAB7L1, MTHFDI, BMI1, COG5, and PDE4D, and upregulated expression of genes KERA and VCY, relative to the level of expression of these genes in an individual who does not have said phenotype, and
wherein the presence of said phenotype indicates that the individual is at low risk of transplant rejection, and

wherein the absence of said phenotype indicates that the individual is at high risk of transplant rejection.

Paragraph 67. A transplant rejection risk assessment system for use in a method as defined in any one of Paragraphs 65 to 66, the system comprising a tool or tools for determining expression of two more genes selected from the group consisting of: KAT2B, CASK, ABCD2, DLG1, SS18, RBL2, RAB7L1, MTHFDI, KERA, BMI1, COG5, PDE4D, and VCY;
and a computer programmed to compute an transplant rejection risk score from the gene expression data of the subject.

Paragraph 68. The method according to any one of Paragraphs 65 to 66, further comprising:
(ii) selecting an individual identified as one who is at low risk of transplant rejection in step (i) for organ and/or tissue transplantation.

Paragraph 69. The method according to any one of Paragraphs 65 to 66, further comprising: (ii) subjecting the individual to organ and/or tissue transplantation if the individual has been identified as one who is at low risk of transplant rejection in step (i).

Paragraph 70. A method for organ and/or tissue transplantation in an individual, the method comprising:
(i) identifying the individual as one who is at low risk of transplant rejection using a method according to any one of Paragraphs 65 to 66, and
(ii) subjecting the individual to organ/and or tissue transplantation.

Paragraph 71. A method for organ and/or tissue transplantation in an individual, the method comprising:
(i) requesting a test providing the results of an analysis to determine the expression level of two more genes selected from the group consisting of: KAT2B, CASK, ABCD2, DLG1, SS18, RBL2, RAB7L1, MTHFDI, KERA, BMI1, COG5, PDE4D, and VCY, in a sample obtained from the individual,
wherein downregulated expression of genes KAT2B, CASK, ABCD2, DLG1, SS18, RBL2, RAB7L1, MTHFDI, BMI1, COG5, and PDE4D, and upregulated expression of genes KERA and VCY, relative to the level of expression of these genes in an individual who does not have an exhausted CD8⁺ T cell or lack of CD4⁺ T cell costimulation phenotype indicates that the individual has said phenotype, and
(ii) subjecting the individual to organ/and or tissue transplantation if the individual has said phenotype.

Paragraph 72. The method according to any one of Paragraphs 65 to 66, further comprising:
(ii) selecting an individual identified as one who is at high risk of transplant rejection in step (i) for treatment prior to organ and/or tissue transplantation, wherein the treatment comprises inducing an exhausted CD8⁺ T cell or lack of CD4⁺ T cell costimulation phenotype in the individual.

Paragraph 73. The method according to any one of Paragraphs 65 to 66, further comprising: (ii) subjecting the individual to treatment prior to organ and/or tissue transplantation if the
individual has been identified as one who is at high risk of transplant rejection in step (i) wherein the treatment comprises inducing an exhausted CD8⁺ T cell or lack of CD4⁺ T cell costimulation phenotype in the individual.

Paragraph 74. A method for treating an individual prior to organ and/or tissue transplantation, the method comprising:

(i) identifying the individual as one who is at high risk of transplant rejection using a method according to any one of Paragraphs 65 to 66, and
(ii) subjecting the individual to treatment to induce an exhausted CD8⁺ T cell or lack of CD4⁺ T cell costimulation phenotype in the individual;
(iii) subjecting the individual to organ/and or tissue transplantation.

Paragraph 75. A method for treating an individual prior to organ and/or tissue transplantation, the method comprising:
(i) requesting a test providing the results of an analysis to determine the expression level of two more genes selected from the group consisting of: KAT2B, CASK, ABCD2, DLG1, SS18, RBL2, RAB7L1, MTHFDI, KERA, BMI1, COG5, PDE4D, and VCY, in a sample obtained from the individual,
wherein upregulated expression of genes KAT2B, CASK, ABCD2, DLG1, SS18, RBL2, RAB7L1, MTHFDI, BMI1, COG5, and PDE4D, and downregulated expression of genes KERA and VCY, relative to the level of expression of these genes in an individual who has an exhausted CD8⁺ T cell or lack of CD4⁺ T cell costimulation phenotype indicates that the individual does not have said phenotype, and
(ii) subjecting the individual to treatment to induce an exhausted CD8⁺ T cell or lack of CD4⁺ T cell costimulation phenotype in the individual if the individual does not have said phenotype;
(iii) subjecting the individual to organ/and or tissue transplantation.

Paragraph 76. The method according to any one of Paragraphs 72 to 75, wherein an exhausted CD8⁺ T cell or lack of CD4⁺ T cell costimulation phenotype is induced in the individual by administering a therapeutically effective amount of a programmed cell death protein 1 (PD-1) ligand.

Paragraph 77. A PD-1 ligand for use in a method of treating an individual prior to organ and/or tissue transplantation, the method comprising
(i) determining whether the individual is at high risk of transplant rejection using a method according to any one of Paragraphs 65 to 66,

(ii) administering therapeutically effective amount of a PD-1 ligand to the individual if the individual is at high risk of infection-associated immunopathology to induce an exhausted CD8⁺ T cell or lack of CD4⁺ T cell costimulation phenotype in the individual, and
(iii) subjecting the individual to organ/and or tissue transplantation.

Paragraph 78. A PD-1 ligand for use according to Paragraph 77, wherein the PD-1 ligand is PDL-I.

Paragraph 79. A method of assessing whether an individual is at high risk or low risk of cancer progression, the method comprising:
(i) determining whether the individual has an exhausted CD8⁺ T cell or lack of CD4⁺ T cell costimulation phenotype using the method of Paragraph 1,

wherein the presence of said phenotype indicates that the individual is at high risk of cancer progression, and
wherein the absence of said phenotype indicates that the individual is at low risk of cancer progression.

Paragraph 80. A method of assessing whether an individual is at high risk or low risk of cancer progression, by determining whether the individual has or does not have an exhausted CD8⁺ T cell or lack of CD4⁺ T cell costimulation phenotype, the method comprising:
(i) providing a PBMC sample obtained from the individual;
(ii) extracting mRNA from the PBMC sample;
(iii) performing reverse transcription quantitative PCR (RT-qPCR) to convert the mRNA into cDNA and determine the expression level of two more genes selected from the group consisting of: KAT2B, CASK, ABCD2, DLG1, SS18, RBL2, RAB7L1, MTHFDI, KERA, BMI1, COG5, PDE4D, and VCY,

wherein said phenotype is characterised by downregulated expression of genes KAT2B, CASK, ABCD2, DLG1, SS18, RBL2, RAB7L1, MTHFDI, BMI1, COG5, and PDE4D, and upregulated expression of genes KERA and VCY, relative to the level of expression of these genes in an individual who does not have said phenotype, and
wherein the presence of said phenotype indicates that the individual is at high risk of cancer progression, and

wherein the absence of said phenotype indicates that the individual is at low risk of cancer progression.

Paragraph 81. A cancer progression risk assessment system for use in a method as defined in any one of Paragraphs 79 to 80, the system comprising a tool or tools for determining expression of two more genes selected from the group consisting of: KAT2B, CASK, ABCD2, DLG1, SS18, RBL2, RAB7L1, MTHFDI, KERA, BMI1, COG5, PDE4D, and VCY;
and a computer programmed to compute an cancer progression risk score from the gene expression data of the subject.

Paragraph 82. The method according to any one of Paragraphs 79 to 80, further comprising:
(ii) selecting an individual identified as one who is at high risk of cancer progression in step (i) for treatment for the cancer.

Paragraph 83. The method according to any one of Paragraphs 79 to 80, further comprising:

(ii) subjecting the individual to treatment for the cancer if the individual has been identified as one who is at high risk of cancer progression in step (i).

Paragraph 84. A method for treating cancer in an individual, the method comprising:
(i) identifying the individual as one who is at high risk of cancer progression using a method according to any one of Paragraphs 79 to 80, and
(ii) subjecting the individual to treatment for the cancer .

Paragraph 85. A method for treating cancer in an individual, the method comprising:
(i) requesting a test providing the results of an analysis to determine the expression level of two more genes selected from the group consisting of: KAT2B, CASK, ABCD2, DLG1, SS18, RBL2, RAB7L1, MTHFDI, KERA, BMI1, COG5, PDE4D, and VCY,
in a sample obtained from the individual,

wherein downregulated expression of genes KAT2B, CASK, ABCD2, DLG1, SS18, RBL2, RAB7L1, MTHFDI, BMI1, COG5, and PDE4D, and upregulated expression of genes KERA and VCY, relative to the level of expression of these genes in an individual who does not have an exhausted CD8⁺ T cell or lack of CD4⁺ T cell costimulation phenotype indicates that the individual has said phenotype, and
(ii) subjecting the individual to treatment for the cancer.

Paragraph 86. The method according to any one of Paragraphs 82 to 85, wherein the treatment comprises inducing a non-exhausted CD8⁺ T cell or CD4⁺ T cell costimulation phenotype in the individual.

Paragraph 87. The method according to Paragraph 86, wherein a non-exhausted CD8⁺ T cell or CD4⁺ T cell costimulation phenotype is induced in the individual by administering a therapeutically effective amount of an inhibitor of programmed cell death protein 1 (PD-1).

Paragraph 88. A PD-1 inhibitor for use in a method of treating cancer in an individual, the method comprising

(i) determining whether the individual is at high risk of cancer progression using a method according to any one of Paragraphs 79 to 80, and
(ii) administering therapeutically effective amount of a PD-1 ligand to the individual if the individual is at high risk of cancer progression to induce a non-exhausted CD8⁺ T cell or CD4⁺ T cell costimulation phenotype in the individual.

Paragraph 89. A method, risk assessment system, PD-1 ligand for use, or PD-1 inhibitor for use according to any one of the preceding Paragraphs, wherein the sample is a whole blood or peripheral blood mononuclear cell (PBMC) sample.

Paragraph 90. A method, risk assessment system, PD-1 ligand for use, or PD-1 inhibitor for use according to any one of the preceding Paragraphs wherein the expression level of said two or more genes is determined using reverse transcription quantitative PCR (RT-qPCR).

Paragraph 91. An in vitro method for identifying a substance capable of inducing an exhausted CD8⁺ T cell phenotype in an individual, the method comprising:
(i) providing a sample of CD8⁺ T cells;
(ii) incubating the CD8⁺ T cells in the presence of anti-CD2, anti-CD3 and anti-CD28 antibodies, IL2, and in the presence or absence of a substance of interest; and
(iii) determining the expression level of IL7R and PD-1 by the CD8⁺ T cells; wherein a lower expression of IL7R and a higher expression of PD-1 by the CD8⁺T cells in the presence of the substance of interest than in the absence of the substance of interest indicates that the substance is capable of inducing an exhausted CD8⁺ T cell phenotype in an individual.

Paragraph 92. An in vitro method for identifying a substance capable of inducing a non-exhausted CD8⁺ T cell phenotype in an individual, the method comprising:
(i) providing a sample of CD8⁺ T cells;
(ii) incubating the CD8⁺ T cells in the presence of anti-CD3 and anti-CD28 antibodies, IL2, and in the presence or absence of a substance of interest; and
(iii) determining the expression level of IL7R and PD-1 by the CD8⁺ T cells; wherein a higher expression of IL7R and a lower expression of PD-1 by the CD8+T cells in the presence of the substance of interest than in the absence of the substance of interest indicates that the substance is capable of inducing a non-exhausted CD8⁺ T cell phenotype in an individual.

Paragraph 93. The method according to Paragraph 91 or 92, wherein the method further comprises formulating a substance identified as capable of inducing an exhausted CD8⁺ T cell phenotype in an individual, or capable of inducing a non-exhausted CD8⁺ T cell phenotype in an individual, into a medicament.

Paragraph 94. A method of preparing CD8⁺T cells with a non-exhausted CD8⁺ T cell phenotype, the method comprising:
(i) providing a sample of CD8⁺ T cells obtained from an individual;
(ii) incubating the CD8⁺ T cells in the presence of anti-CD2, anti-CD3 and anti-CD28 antibodies, and IL2; and
(iii) determining the expression level of IL7R and PD-1 by the CD8⁺ T cells; wherein a higher expression of IL7R and a lower expression of PD-1 by the CD8+T cells following incubation in the presence of the anti-CD2, anti-CD3 and anti-CD28 antibodies, and IL2 compared with prior to incubation, indicates that the CD8⁺ T cells have a non-exhausted CD8⁺ T cell phenotype.

Paragraph 95. A method of preparing CD8⁺T cells with an exhausted CD8⁺ T cell phenotype, the method comprising:
(i) providing a sample of CD8⁺ T cells obtained from an individual;
(ii) incubating the CD8⁺ T cells in the presence of anti-CD3 and anti-CD28 antibodies, and IL2; and
(iii) determining the expression level of IL7R and PD-1 CD8⁺ T cells;
wherein a lower expression of IL7R and a higher expression of PD-1 by the CD8⁺T cells following incubation in the presence of the anti-CD3 and anti-CD28 antibodies, and IL2 compared with prior to incubation, indicates that the CD8⁺ T cells have an exhausted CD8⁺ T cell phenotype.

Paragraph 96. The method according to Paragraph 94 or 95, wherein said method further comprises administering the CD8⁺ T cells to the individual from which the CD8⁺ T cells were obtained.

Paragraph 97. A kit for assessing whether an individual has an exhausted CD8⁺ T cell or lack of CD4⁺ T cell costimulation phenotype, or whether an exhausted CD8⁺ T cell or lack of CD4⁺ T cell costimulation phenotype is present in a sample of CD8⁺ and CD4⁺ T cells, wherein said kit comprises reagents for establishing the expression level of two or more genes selected from the group consisting of: KAT2B, CASK, ABCD2, DLG1, SS18, RBL2, RAB7L1, MTHFD1, KERA, BMI1, COG5, PDE4D, and VCY.

### BRIEF DESCRIPTION OF THE FIGURES

FIG. 1: T cell costimulation with CD2 prevents development of an exhausted IL7R^{l0}PD1^{hi} phenotype. (A) Schematic of the magnetic bead system providing variable TCR signal duration/costimulation during in vitro culture. (B-D) Linear plots showing IL7R^{hi} population resulting from (B) 36h (top line [at endpoint]) v 6d (bottom line [at endpoint]) anti-CD3/28 stimulation, (C) 6d anti-CD2/3/28 (top line) v 6d anti-CD3/28 (bottom line) and (D) from 6d anti-CD2/3/28 with (bottom line) and without (top line) Fc-PDL1.

FIG. 2: A surrogate marker of CD4⁺ T cell costimulation/lack of CD8⁺ T cell exhaustion in PBMC gene expression data correlates with clinical outcome in chronic viral infection, vaccination, infection and autoimmunity. (A) Scatterplot showing the top 100 genes ranked by ability to identify CD4 T cell costimulation subgroups in PBMC data. x-axis=variable importance. (B) Kaplan-Meier plots showing censored flare-free survival stratified by expression of KAT2B (above or below median) in AAV and SLE patients (n=37, training set) replicated on Affymetrix GeneST1.0 and in an independent cohort (test set, n=47), P=log-rank test. (C) Line and scatterplots showing serial KAT2B expression (n=54) following therapy of chronic HCV infection giving a marked (n=28) or poor response (n=26). P=2-way ANOVA. (D) Boxplot showing post-vaccine malaria protection in a clinical trial (n=43) stratified by KAT2B expression (above or below median), P=Fisher's exact test. (E) Boxplot showing % protection (black) in vaccinees (n=28) following seasonal influenza vaccine stratified by KAT2B expression, P=Fisher's exact test (F) Scatterplot showing neutralizing antibody titer following YF-17D vaccination (yellow fever vaccination), stratified by KAT2B expression (F, above or below median). P=Mann-Whitney test. (G) Line and scatterplot showing serial KAT2B expression throughout dengue infection (n=78) stratified by progression to hemorrhagic fever (DHF, n=24) or uncomplicated course (UD, n=54). x-axis=time (days) relative to defervescence. (H) Boxplot showing % IPF patients (n=75) progressing to transplantation/death (black) stratified by KAT2B expression (above or below median). P=Fisher's exact test. (I-K) Scatterplots showing serial KAT2B expression in healthy age, sex and HLA-matched controls (I) and in pre-T1D cases (n=5), 2 of which seroconvert to islet-cell antibodies (J, black line) and 3 of which develop T1D (K, black line).

FIG. 3: Top PBMC surrogate markers reflect expression of CD4⁺ T cell costimulation/CD8⁺ T cell exhaustion modules within CD4⁺ T cell and CD8⁺ T cell data respectively. Top PBMC-level predictors (n=13) were selected as indicated in Fig4A and data is shown comparing expression of the optimal predictor (KAT2B, A, D) and of each other top predictor gene (C, F) in PBMC data compared to expression of the CD4 costimulation module eigengene in CD4 data (A-C) and the CD8 exhaustion signature eigengene in CD8 data (D-F) for n=44 patients with AAV. Significance of correlation, *P<0.05, **P<0.01, ***P<0.001. (B, E) Scatterplots showing the outcome of multiple linear regression models testing the association of KAT2B expression in CD4 (B) and CD8 (F) data (dot at top right of figures) directly compared to clinical markers of disease activity (other dots). x-axis=magnitude of association (regression coefficient, change in normalized flare rate (flares/days follow-up) per unit change in each variable tested). y-axis =significance of association in multiple regression model, P. significance threshold (dashed line, P=0.05). Clinical variables incorporated=disease activity score (BVAS), CRP, Lymphocyte count, neutrophil count, IgG. As expected, surrogate markers showed stronger correlation with the CD4 than the CD8 signature as the algorithm was trained to detect the CD4 costimulation module.

FIG. 4: Hierarchical clustering of multiple datasets using 13 top PBMC-level surrogate markers of an exhausted CD8⁺ T cell/lack of CD4⁺ T cell costimulation phenotype identifies patient subgroups with distinct clinical outcomes. Replication of association between surrogate markers of an exhausted CD8⁺ T cell/lack of CD4⁺ T cell costimulation phenotype signatures and clinical outcome (as shown in FIG. 2 C-K) but using all top 13 PBMC-level surrogates rather than KAT2B alone. Clinical outcome associated with each subgroup identified is shown in A (HCV, % responders to IFNa/ribavirin therapy), B (% showing protection versus no protection from malaria vaccine), C (% response to influenza vaccination), D (yellow fever antibody-titer post-vaccination), E (% progression to dengue hemhorrhagic fever, DHF), F (% of idiopathic pulmonary fibrosis patients progressing to need for transplantation or death) and G (% samples from patients with prior or subsequent progression to islet-cell antibody seroconversion or to a diagnosis of T1D). Groups 1 and 2 in FIG. 4 refer to individuals with a non-exhausted CD8⁺ T cell/CD4⁺ T cell costimulation phenotype, and to individuals with an exhausted CD8⁺ T cell/lack of CD4⁺ T cell costimulation phenotype, respectively.

Further aspects and embodiments of the invention will be apparent to those skilled in the art given the present disclosure including the following experimental exemplification.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above.

### DETAILED DESCRIPTION OF THE INVENTION

The present inventors have identified a gene expression signature that can be used to identify an exhausted CD8⁺ T cell or lack of CD4⁺ T cell costimulation phenotype in a sample comprising CD8⁺ and CD4⁺ T cells, such as a PBMC or whole blood sample. Specifically, the present inventors have discovered that an exhausted CD8⁺ T cell or lack of CD4⁺ T cell costimulation phenotype is characterised by downregulated expression of genes KAT2B, CASK, ABCD2, DLG1, SS18, RBL2, RAB7L1, MTHFDI, BMI1, COG5, and PDE4D, and upregulated expression of genes KERA and VCY. The GenBank accession numbers and version numbers for these genes are set out in Table 1. The present inventors have also discovered that an exhausted CD8⁺ T cell or lack of CD4⁺ T cell costimulation phenotype is indicative of whether an individual is:
at low risk of: autoimmune disease progression, infection-associated immunopathology, or transplant rejection; and

at high risk of: chronic infection progression or not responding to a treatment for a chronic infection, not mounting an effective immune response to a vaccine, or cancer progression.

A method disclosed herein, such as a method of assessing whether an individual has an exhausted CD8⁺ T cell or lack of CD4⁺ T cell costimulation phenotype, or whether CD8⁺ and CD4⁺ T cells in a sample have an exhausted CD8⁺ T cell or lack of CD4⁺ T cell costimulation phenotype, or identifying a substance capable of inducing an exhausted CD8⁺ T cell or lack of CD4⁺ T cell costimulation phenotype in an individual, may comprising determining the expression level of two more genes selected from the group consisting of KAT2B, CASK, ABCD2, DLG1, SS18, RBL2, RAB7L1, MTHFDI, BMI1, COG5, and PDE4D, KERA and VCY. Determining the expression level of two or more of said genes is expected to be more robust than determining the expression level of only a single gene, such as KAT2B alone. For example, determining the expression level of two or more genes may allow the presence, or absence, of an exhausted CD8⁺ T cell or lack of CD4⁺ T cell costimulation phenotype to be accurately determined even if the expression level of e.g. one gene cannot be determined, or is inaccurate. Genes KAT2B, CASK, ABCD2, DLG1, SS18, RBL2, RAB7L1, MTHFDI, BMI1, COG5, PDE4D, KERA, and VCY represent the top 13 marker genes for determining the presence, or absence, of an exhausted CD8⁺ T cell or lack of CD4⁺ T cell costimulation phenotype, as identified by the present inventors.

For example, a method disclosed herein, may comprise determining the expression level of three or more, four or more, five or more, six or more, seven or more, eight or more, nine or more, ten or more, eleven or more, twelve or more, or all thirteen genes selected from the group consisting of KAT2B, CASK, ABCD2, DLG1, SS18, RBL2, RAB7L1, MTHFDI, BMI1, COG5, and PDE4D, KERA and VCY. Preferably, a method disclosed herein comprises determining the expression level of KAT2B and one or more genes selected from the group consisting of CASK, ABCD2, DLG1, SS18, RBL2, RAB7L1, MTHFDI, BMI1, COG5, and PDE4D, KERA and VCY. For example, a method disclosed herein may comprise determining the expression level of KAT2B and two or more, three or more, four or more, five or more, six or more, seven or more, eight or more, nine or more, ten or more, eleven or more, or all twelve genes selected from the group consisting of CASK, ABCD2, DLG1, SS18, RBL2, RAB7L1, MTHFDI, BMI1, COG5, and PDE4D, KERA and VCY.

Exhausted CD8⁺ T Cell or Lack of CD4⁺ T Cell Costimulation Phenotypes

An individual who has an exhausted CD8⁺ T cell or lack of CD4⁺ T cell costimulation phenotype has downregulated expression of genes KAT2B, CASK, ABCD2, DLG1, SS18, RBL2, RAB7L1, MTHFDI, BMI1, COG5, and PDE4D, and upregulated expression of genes KERA and VCY compared with an individual who does not have an exhausted CD8⁺ T cell or lack of CD4⁺ T cell costimulation phenotype. An upregulated or downregulated expression of a gene preferably refers to a significantly upregulated, or significantly downregulated, level of expression of said gene, respectively. An individual who does not have an exhausted CD8⁺ T cell or lack of CD4⁺ T cell costimulation phenotype, may also be referred to as an individual who has a non-exhausted CD8⁺ T cell or CD4⁺ T cell costimulation phenotype. Whether an individual has an upregulated or downregulated level of expression of the genes in question may be determined by any convenient means and many suitable techniques are known in the art and described herein.

As is the case with most biomarkers, accuracy of prediction may not be absolute. An individual who is at high risk of autoimmune disease progression, progression of a chronic infection, not responding to a treatment for a chronic infection, not mounting an effective immune response to vaccination, infection-associated immunopathology, transplant rejection, or cancer progression, may therefore have a 50% or greater, 60% or greater, 70% or greater, 80% or greater, or 90% or greater chance of autoimmune disease progression, cancer progression, progression of a chronic infection, not responding to a treatment for a chronic infection, not mounting an effective immune response to vaccination, or transplant rejection than an individual who does not have a high risk CD8⁺ T cell/CD4⁺ T cell phenotype. Similarly an individual who is at low risk of autoimmune disease progression, cancer progression, progression of a chronic infection, not responding to a treatment for a chronic infection, not mounting an effective immune response to vaccination, or transplant rejection may have a 55% or lower, 50% or lower, 40% or lower, 30% or lower, 20% or lower, or 10% or lower chance of autoimmune disease progression, cancer progression, progression of a chronic infection, not responding to a treatment for a chronic infection, not mounting an effective immune response to vaccination, or transplant rejection than an individual who has a high risk CD8⁺ T cell/CD4⁺ T cell phenotype.

There are many suitable methods which may be used to determine whether an individual, or cell sample, has upregulated or downregulated expression of two or more genes selected from the group consisting of: KAT2B, CASK, ABCD2, DLG1, SS18, RBL2, RAB7L1, MTHFDI, BMI1, COG5, and PDE4D, KERA, and VCY.

For example, the level of expression of two or more genes selected from the group consisting of KAT2B, CASK, ABCD2, DLG1, SS18, RBL2, RAB7L1, MTHFDI, BMI1, COG5, PDE4D, KERA and VCY in a sample, which may be a sample obtained from an individual, (i.e. the test sample) may be compared with a threshold level for each gene in question. A threshold level for a gene can be determined using qPCR expression data and network modelling (for example using support vector machines, principal component or adaptive elastic net approaches) to establish optimal expression thresholds that allow maximal, optimal separation of our existing cohorts into discrete prognostic subgroups. Comparison of the level of expression of two or more genes selected from the group consisting of KAT2B, CASK, ABCD2, DLG1, SS18, RBL2, RAB7L1, MTHFDI, BMI1, COG5, PDE4D, KERA and VCY with threshold levels for the genes in question may indicate whether the individual has or does not have an exhausted CD8⁺ T cell or lack of CD4⁺ T cell costimulation phenotype.

Alternatively, the control may be the median expression of the genes in question (i.e. two or more genes selected from the group consisting of KAT2B, CASK, ABCD2, DLG1, SS18, RBL2, RAB7L1, MTHFDI, BMI1, COG5, PDE4D, KERA and VCY) in samples obtained from a group of individuals, wherein the group comprised individuals, preferably at least 100, at least 50, or at least 10 individuals, who did not have autoimmune disease progression, did not have progression of a chronic infection, did not respond to a treatment for a chronic infection, did not mount an effective immune response to vaccination, did not develop infection-associated immunopathology, did not experience transplant rejection, or had cancer progression, as applicable. In this case, an equal or below median expression of genes KAT2B, CASK, ABCD2, DLG1, SS18, RBL2, RAB7L1, MTHFDI, BMI1, COG5, or PDE4D, or an equal or above median expression level of genes KERA or VCY in a sample may indicate the presence of an exhausted CD8⁺ T cell or lack of CD4⁺ T cell costimulation phenotype, while an above median expression genes KAT2B, CASK, ABCD2, DLG1, SS18, RBL2, RAB7L1, MTHFDI, BMI1, COG5, or PDE4D, or below median expression level of genes KERA or VCY in a sample may indicate the absence of an exhausted CD8⁺ T cell or lack of CD4⁺ T cell costimulation phenotype.

As a further alternative, the control may the median expression of the genes in question in samples obtained from a group of individuals, preferably at least 100, at least 50, or at least 10 individuals, wherein the group comprised individuals who had autoimmune disease progression, did not have progression of a chronic infection, responded to a treatment for a chronic infection, mounted an effective immune response to a vaccine, developed infection-associated immunopathology, experienced transplant rejection, or did not experience cancer progression, as applicable. In this case, a below median expression level of genes KAT2B, CASK, ABCD2, DLGI, SS18, RBL2, RAB7L1, MTHFDI, BMI1, COG5, or PDE4D, or an above median expression level of genes KERA or VCY in a sample may indicate the presence of an exhausted CD8⁺ T cell or lack of CD4⁺ T cell costimulation phenotype, while an equal or above median expression level of genes KAT2B, CASK, ABCD2, DLG1, SS18, RBL2, RAB7L1, MTHFDI, BMI1, COG5, or PDE4D, or an equal or below median expression level of genes KERA or VCY in a sample may indicate the absence of an exhausted CD8⁺ T cell or lack of CD4⁺ T cell costimulation phenotype.

As a yet further alternative, the control may be the median expression of the genes in question in samples obtained from a group of individuals, preferably at least 100, at least 50, or at least 10 individuals, wherein the group comprised individuals who did and did not have autoimmune disease progression, did and did not have cancer progression, did and did not have progression of a chronic infection, did and did not respond to treatment for a chronic infection, did and did not mount an effective immune response to vaccination, did and did not develop infection-associated immunopathology, or did and did not experience transplant rejection, as applicable. Preferably the group comprised an equal number, or essentially equal number, of individuals who did and did not have autoimmune disease progression, cancer progression, progression of a chronic infection, respond to a treatment for a chronic infection, mount an effective immune response to vaccination, develop infection-associated immunopathology, or experience transplant rejection, as applicable. In this case, a below median expression level of genes KAT2B, CASK, ABCD2, DLG1, SS18, RBL2, RAB7L1, MTHFDI, BMI1, COG5, or PDE4D, or above median expression level of genes KERA or VCY in a sample may indicate the presence of an exhausted CD8⁺ T cell or lack of CD4⁺ T cell costimulation phenotype, while an above median expression level of genes KAT2B, CASK, ABCD2, DLG1, SS18, RBL2, RAB7L1, MTHFDI, BMI1, COG5, or PDE4D, or below median expression level of genes KERA or VCY in a sample may indicate the absence of an exhausted CD8⁺ T cell or lack of CD4⁺ T cell costimulation phenotype.

In the case of type 1 diabetes the control sample may have been obtained from an individual who did not progress to type 1 diabetes. The control sample may have been obtained from an individual who did not develop type 1 diabetes-associated autoantibodies. Preferably, the control sample was obtained from an individual with the same genetic predisposition to type 1 diabetes as the individual from which the test sample was obtained. Most preferably, the control sample was obtained from an individual with the same high risk HLA haplotype, as the individual from which the test sample was obtained. The control sample may have been obtained from an individual with no first degree relatives with type 1 diabetes. Preferably, the individuals in the group were the same age, as the individual from which the test sample was obtained.

### Determining Gene Expression

The level of expression of genes KAT2B, CASK, ABCD2, DLG1, SS18, RBL2, RAB7L1, MTHFDI, BMI1, COG5, and PDE4D, KERA and VCY may be determined by any convenient means and many suitable techniques are known in the art. For example, suitable techniques include: reverse-transcription quantitative PCR (RT-qPCR), microarray analysis, enzyme-linked immunosorbent assays (ELISA), protein chips, flow cytometry (such as Flow-FISH for RNA, also referred to as FlowRNA), mass spectrometry, Western blotting, and northern blotting. A method of the invention may therefore comprise bringing a sample obtained from an individual into contact with a reagent suitable for determining the expression level of KAT2B, CASK, ABCD2, DLG1, SS18, RBL2, RAB7L1, MTHFDI, BMI1, COG5, and PDE4D, KERA and/or VCY, e.g. a reagent or reagents suitable for determining the expression level of one or more of said genes using RT-qPCR, microarray analysis, ELISA, protein chips, flow cytometry, mass spectrometry, or Western blotting. For example, the reagent may be a pair or pairs of nucleic acid primers, suitable for determining the expression level of one or more of said genes using RT-qPCR. Alternatively, the reagent may be an antibody suitable for determining the expression level of said one or more genes using ELISA or Western blotting. Preferably, the level of expression of said genes is determined using RT-qPCR or Flow-FISH. More preferably, the level of expression of said genes is determined using RT-qPCR.

RT-qPCR allows amplification and simultaneous quantification of a target DNA molecule. To analyze gene expression levels using RT-qPCR, the total mRNA of a PBMC or whole blood sample may first be isolated and reverse transcribed into cDNA using reverse transcriptase. For example, mRNA levels can be determined using e.g. Taqman Gene Expression Assays (Applied Biosystems) on an ABI PRISM 7900HT instrument according to the manufacturers instructions. Transcript abundance can then be calculated by comparison to a standard curve.

Flow-FISH for RNA employs flow cytometry to determine the abundance of a target mRNA within a sample using fluorescently-tagged RNA oligos. This technique is described, for example, in Porichis et al., Nat Comm (2014) 5:5641. The advantage of this technique is that it can be used without the need to separate the cells present in a sample.

Microarrays allow gene expression in two samples to be compared. Total RNA is first isolated from, e.g. PBMCs or whole blood using, for example, Trizol or an RNeasy mini kit (Qiagen). The isolated total RNA is then reverse transcribed into double-stranded cDNA using reverse transcriptase and polyT primers and labelled using e.g. Cy3- or Cy5-dCTP. Appropriate Cy3- and Cy5-labelled samples are then pooled and hybridised to custom spotted oligonucleotide microarrays comprised of probes representing suitable genes and control features, such as the microarray described in (Willcocks et al., J Exp Med 205, 157382, 2008). Samples may be hybridised in duplicate, using a dye-swap strategy, against a common reference RNA derived from pooled PBMC or whole blood samples. Following hybridisation, arrays are washed and scanned on e.g. an Agilent G2565B scanner. Suitable alternatives to the steps described above are well known in the art and would be apparent to the skilled person. The raw microarray data obtained can then be analyzed using suitable methods to determine the relative expression of genes KAT2B, CASK, ABCD2, DLG1, SS18, RBL2, RAB7L1, MTHFD1, BMI1, COG5, and PDE4D, KERA and VCY.

Enzyme-linked immunosorbent assays (ELISAs) allow the relative amounts of proteins present in a sample to be detected. The sample is first immobilized on a solid support, such as a polystyrene microtiter plate, either directly or via an antibody specific for the protein of interest. After immobilization, the antigen is detected using an antibody specific for the target protein. Either the primary antibody used to detect the target protein may be labelled to allow detection, or the primary antibody can be detected using a suitably labelled secondary antibody. For example, the antibody may be labelled by conjugating the antibody to a reporter enzyme. In this case, the plate developed by adding a suitable enzymatic substrate to produce a visible signal. The intensity of the signal is dependent on the amount of target protein present in the sample.

Protein chips, also referred to as protein arrays or protein microarrays, allow the relative amounts of proteins present in a sample to be detected. Different capture molecules may be affixed to the chip. Examples include antibodies, antigens, enzymatic substrates, nucleotides and other proteins. Protein chips can also contain molecules that bind to a range of proteins. Protein chips are well known in the art and many different protein chips are commercially available.

Western blotting also allows the relative amounts of proteins present in a sample to be detected. The proteins present in a sample are first separated using gel electrophoresis. The proteins are then transferred to a membrane, e.g. a nitrocellulose or PVDF membrane, and detected using monoclonal or polyclonal antibodies specific to the target protein. Many different antibodies are commercially available and methods for making antibodies to a given target protein are also well established in the art. To allow detection, the antibodies specific for the protein(s) of interest, or suitable secondary antibodies, may, for example, be linked to a reporter enzyme, which drives a colorimetric reaction and produces a colour when exposed to an appropriate substrate. Other reporter enzymes include horseradish peroxidase, which produces chemiluminescence when provided with an appropriate substrate. Antibodies may also be labelled with suitable radioactive or fluorescent labels. Depending on the label used, protein levels may be determined using densitometry, spectrophotometry, photographic film, X-ray film, or a photosensor.

Flow cytometry allows the relative amounts of proteins present in e.g. a PBMC or whole blood sample obtained from a subject to be determined. Flow cytometry can also be used to detect or measure the level of expression of a protein of interest on the surface of cells. Detection of proteins and cells using flow cytometry normally involves first attaching a fluorescent label to the protein or cell of interest. The fluorescent label may for example be a fluorescently-labeled antibody specific for the protein or cell of interest. Many different antibodies are commercially available and methods for making antibodies specific for a protein of interest are also well established in the art.

Mass spectrometry, e.g. matrix-assisted laser desorption/ionization (MALDI) mass spectrometry, allows the identification of proteins present in a sample obtained from a individual using e.g. peptide mass finger printing. Prior to mass spectrometry the proteins present in the sample may be isolated using gel electrophoresis, e.g. SDS-PAGE, size exclusion chromatography, or two-dimensional gel electrophoresis.

In the methods described herein, the expression level of two or more genes selected from the group consisting of KAT2B, CASK, ABCD2, DLG1, SS18, RBL2, RAB7L1, MTHFDI, BMI1, COG5, and PDE4D, KERA and VCY may be determined in an individual, e.g. in a sample obtained from an individual, to assess whether the individual has an exhausted CD8⁺ T cell or lack of CD4⁺ T cell costimulation phenotype.

Kit of Parts

Also disclosed is a kit for use in assessing whether an individual has an exhausted CD8⁺ T cell or lack of CD4⁺ T cell costimulation phenotype, or whether an exhausted CD8⁺ T cell or lack of CD4⁺ T cell costimulation phenotype is present in a sample comprising CD8⁺ and/or CD4⁺ T cells. The kit comprises reagents for establishing the expression level of two or more genes selected from the group consisting of: KAT2B, CASK, ABCD2, DLG1, SS18, RBL2, RAB7L1, MTHFDI, KERA, BMI1, COG5, PDE4D, and VCY. The GenBank accession numbers and version numbers for these genes are set out in Table I.The kit may comprise reagents for establishing the expression level of three or more, four or more, five or more, six or more, seven or more, eight or more, nine or more, ten or more, eleven or more, twelve or more, or all thirteen of the genes selected from the group consisting of: KAT2B, CASK, ABCD2, DLG1, SS18, RBL2, RAB7L1, MTHFDI, KERA, BMI1, COG5, PDE4D, and VCY. Preferably, the kit comprises reagents for establishing the expression level of KAT2B, along with reagents for establishing the expression level of one or more, two or more, three or more, four or more, five or more, six or more, seven or more, eight or more, nine or more, ten or more, eleven or more, or all 12 genes selected from the group consisting of CASK, ABCD2, DLG1, SS18, RBL2, RAB7L1, MTHFDI, KERA, BMI1, COG5, PDE4D, and VCY.

The reagents may be reagents suitable for establishing the expression KAT2B using RT-qPCR, microarray analysis, ELISA, and/or western blotting. For example, the kit may comprise primers suitable for establishing the level of expression of KAT2B, using e.g. RT-qPCR. The design of suitable primers is routine and well within the capabilities of the skilled person. In addition to detection reagents, a kit may include one or more articles and/or reagents for performance of the method, such as buffer solutions, and/or means for obtaining the test sample itself, e.g. means for obtaining and/or isolating a sample and sample handling containers (such components generally being sterile). The kit may include instructions for use of the kit in a method for assessing whether an individual has an exhausted CD8⁺ T cell or lack of CD4⁺ T cell costimulation phenotype, or whether an exhausted CD8⁺ T cell or lack of CD4⁺ T cell costimulation phenotype is present in a sample of CD8⁺ and CD4⁺ T cells.

Sample

One advantage of the genes whose expression is determined in the present invention is that expression of these genes can be determined in unseparated peripheral blood mononuclear cells (PBMCs) or whole blood. Thus, a sample, as referred to in the context of the present invention may be a PBMC or whole blood sample.

Individual

“Individual” refers to a human individual. An individual may also be referred to as a patient, i.e. a human patient.

Autoimmune Disease

Autoimmune disease is common, affecting about 10% of the population. Management of autoimmune diseases usually involves immunosuppressive therapy which, although often effective, can result in infection which is a significant cause of morbidity and mortality associated with these diseases. Many autoimmune diseases present with an initial acute phase followed by sporadic relapses rather than a continuous disease progression. Treatment usually involves an initial period of intensive treatment, referred to as induction therapy, during the first presentation of the disease followed by maintenance therapy, which is aimed at preventing relapses. However, disease progression varies widely between individuals, ranging from those that have frequent relapses after the initial acute phase to those which have no relapses at all.

Given the substantive morbidity and mortality associated with immunosuppressive therapy, it would be advantageous if patients unlikely to have relapses of the disease in question could be identified. Identification of these patients would allow clinicians to reduce the immunosuppressive maintenance therapy for these individuals, or even stop it completely, with a corresponding decrease in the morbidity and mortality associated with this form of treatment. In addition, individuals likely to have frequent relapses may benefit from a more intensive form of maintenance therapy, which would not be justified if given to all patients indiscriminately due to the severity of the likely side effects.

Although many autoimmune diseases present with heterogeneous clinical features in the clinic, it is not possible, on the basis of these clinical features, to determine what the likely pattern of disease progression fora given patient will be, and a number of tests have been developed with a view to addressing this problem. For example, in the case of the autoimmune disease anti-neutrophil cytoplasmic antibody (ANCA)-associated vasculitis, two autoimmune antibodies, one directed against proteinase-3 (PR-3), the other against myeloperoxidase (MPO), have been identified. However, although statistically the presence of anti-proteinase 3 antibodies is associated with disease progression, the association is not sufficiently strong to allow treatment decisions to be made based on the detection of these antibodies. In the case of SLE, the titre of anti-double stranded DNA antibodies has been used to predict disease progression. However, again the association of these antibodies with disease progression is not sufficiently strong to determine therapy. In addition, the present inventors have previously described a method of predicting autoimmune disease flare on the basis of biomarkers as described in W02010/084312.

However, there remains a need in the art for accurate methods of predicting disease progression in autoimmune diseases in order to avoid excess morbidity and mortality as a result of unnecessary immunosuppressive therapy.

The present inventors have discovered that upregulated expression of genes KAT2B, CASK, ABCD2, DLG1, SS18, RBL2, RAB7L1, MTHFD1, BMI1, COG5, and PDE4D, and downregulated expression of genes KERA and VCY, relative to a control indicates that an individual does not have an exhausted CD8⁺ T cell or lack of CD4⁺ T cell costimulation phenotype. Furthermore, the absence of this phenotype in an individual indicates that the individual is at high risk of autoimmune disease progression, while the presence of this phenotype in an individual is at low risk of autoimmune disease progression.

The risk of progression has important implications for the optimal management of the autoimmune disease in these individuals. For the individuals with this phenotype, any benefits of immunosuppressive maintenance therapy may not outweigh the associated increase in morbidity and mortality. In contrast, individuals without this phenotype are likely to benefit substantially from immunosuppressive maintenance therapy, and the benefits are likely to outweigh the risks. In addition, individuals without this phenotype may benefit from more intensive treatment than is usual during the maintenance phase but which would not be justified if given to all individuals indiscriminately due to the severity of the likely side effects of such treatment.

“Autoimmune disease” refers to any condition which involves an overactive immune response of the body against substances and tissues normally present in the body. The autoimmune disease is preferably an autoimmune disease wherein the presence of an exhausted CD8⁺ T cell or lack of CD4⁺ T cell costimulation phenotype indicates that the individual is at low risk of autoimmune disease progression and wherein the absence of said phenotype is at high risk of autoimmune disease progression. Autoimmune diseases of particular interest include type 1 diabetes, idiopathic pulmonary fibrosis (IPF), systemic lupus erythematosus (SLE), and vasculitis, such as ANCA-associated vasculitis (AAV). The autoimmune disease is preferably not rheumatoid arthritis (RA) or inflammatory bowel disease (IBD).

“Autoimmune disease progression” refers to the progression of the autoimmune disease after initial presentation of the disease in an individual. For example, autoimmune disease progression may refer to relapses, or flares, of the autoimmune disease experienced by the individual after initial presentation of the autoimmune disease. A relapse or flare may be an event that requires increased therapy, e.g. increased immunosuppressive therapy or surgery. SLE and AAV are characterised by relapses and flares. A high risk of autoimmune disease progression may accordingly refer to a high risk that the individual will experience relapses or flares of the disease after initial presentation, while a low risk of autoimmune disease progression may refer to a low risk that the individual will experience relapses or flares of the disease after initial presentation. Autoimmune disease progression may also refer to an ongoing worsening of clinical features, which can occur in the absence of discrete flares. In the case of IPF, ongoing worsening of clinical features may result in lung transplantation or death. An ongoing worsening of clinical features in the case of IPF may refer to an ongoing reduction in lung function. Methods for measuring lung function are known in the art and include spirometry (principally FVC), TLCO, A—a gradient and transitional dyspnoea index, effort tolerance, hospitalisation indices, imaging (e.g. chest X ray, high resolution CT scan (HRCT) and FDG-PET scanning), and invasive assessments (e.g. bronchoalveolar lavage and lung biopsy). A high risk of autoimmune disease progression may accordingly refer to a high risk that the individual will experience an ongoing worsening of clinical features after initial presentation, while a low risk of autoimmune disease progression may refer to a low risk that the individual will experience an ongoing worsening of clinical features after initial presentation.

Autoimmune disease progression may also refer to progression to overt disease, such as in the case of type 1 diabetes. A high risk of autoimmune disease progression may accordingly refer to a high risk that the individual will progress to overt disease, while a low risk of autoimmune disease progression may refer to a low risk that the individual will progress to overt disease. Autoimmune disease progression may also refer to an event requiring increased therapy in the form of either increased immunosuppression or surgery. Such events included relapses, or flares, of the disease after a period of remission, as well as instances where the disease does not enter remission in response to initial therapy and increased immunosuppression or surgery is required as a result. A high risk of autoimmune disease progression may accordingly refer to a high risk that the individual will experience events requiring increased therapy after initial presentation of the disease, while a low risk of autoimmune disease progression may refer to a low risk that the individual will experience events requiring increased therapy after initial presentation of the disease.

Type 1 diabetes, also known as diabetes mellitus type 1 or juvenile diabetes, is an autoimmune disease caused by selective destruction of insulin-producing [3 cells in the islets of Langerhans (Elo et al., 2010, J Autoimmun. 35, 70-76). The incidence rate varies by geographic region, with 8-17 cases per 100,000 in Northern Europe and the US and 1-3 case per 100,000 in China and Japan. A particularly high incidence rate is seen in Scandinavian countries, with 35 cases per 100,000. Incidence rates are increasing, especially in the Western world (Elo et al., 2010), and it is estimated that 11-22 million individuals worldwide are currently living with type 1 diabetes.

Type 1 diabetes is usually treated with lifelong insulin replacement therapy, accompanied by dietary management and monitoring of glucose levels. Serious complications of type 1 diabetes are common, especially where the disease is poorly managed. These include heart disease, strokes, nerve damage, retinopathy, kidney disease and kidney failure, as well as miscarriage and stillbirth in pregnant women with diabetes. Occasionally, pancreas transplants are used to cure diabetes but as this requires lifelong immunosuppressive therapy, which is more dangerous than insulin replacement therapy, this is normally only a viable option for individuals also requiring kidney transplants due to kidney failure. Other therapeutic approaches which are being trialed include islet cell transplantation and stem cell educator therapies. However, at present there is no practicable cure for type 1 diabetes.

There is therefore a need for strategies for preventing the development of type 1 diabetes in high-risk individuals (Elo et al., 2010). This requires identifying individuals at high risk of developing the disease before the onset of the disease. Several genetic risk factors for type 1 diabetes are known. These include high-risk HLA haplotypes, such as DRB1*0401, DRB1*0402, DRB1*0405, DQA*0301, DQB1*0302 and DQB1*0201. A first degree relative with type 1 diabetes also increases the risk of a child developing type 1 diabetes. However, while all of these risk factors increase the risk of an individual developing the disease, they are, in the majority of cases, not sufficiently predictive to allow individuals with a given risk factor or risk factors to be subjected to preventative therapy indiscriminately. For example, the risk of a child developing type 1 diabetes is about 10% if the father or a sibling has type 1 diabetes and about 1-4% if the mother has type 1 diabetes.

Hence, there is a need for biomarkers which can be used to predict whether an individual, who is genetically predisposed to developing type 1 diabetes, will develop type 1 diabetes prior to the onset of the disease. This would provide a window for treating these individuals with preventative therapy.

Progression to clinical type 1 diabetes can be monitored by the appearance of autoantibodies against e.g. islet cells (ICA), insulin (IAA), protein tyrosine phosphatase-related IA-2 protein (IA-2A), glutamic decarboxylase (GADA), and cation efflux transporter ZnT8, which are considered to signify the initiation of autoimmunity (Elo et al., 2010). However, it would be advantageous to identify individuals who will progress to type 1 diabetes even before the initiation of autoimmunity. Furthermore, detection of autoantibodies is, in most cases, not 100% predictive of the individual progressing to type 1 diabetes, nor does it indicate how soon onset of the disease will occur.

There therefore remains a need in the art for methods that allow the identification of individuals who will develop type 1 diabetes prior to the appearance of autoantibodies, as well as biomarkers for predicting likelihood of progression to clinical type 1 diabetes in individuals with detectable autoantibodies.

The present inventors have discovered that the presence or absence of an exhausted CD8⁺ T cell or lack of CD4⁺ T cell costimulation phenotype in an individual genetically predisposed to type 1 diabetes is indicative of whether the individual is at low or high risk of progressing to type 1 diabetes, respectively.

Thus, provided herein are methods which may be used to assess whether and individual who is genetically predisposed to type 1 diabetes is at high risk or at low risk of progressing to type 1 diabetes.

An individual who is genetically predisposed to type 1 diabetes may have a high-risk HLA (human leukocyte antigen) haplotypes type. Such haplotypes are disclosed in Erlich et al., Diabetes (2008) 57:1084, and include DRB1*0301-DQA1*0501-DQB1*0201 (OR 3.64), DRB1*0405-DQA1*0301-DQB1*0302 (OR 11.37), DRB1*0401-DQA1*0301-DQB1*0302 (OR 8.39), DRB1*0402-DQA1*0301-DQB1*0302 (OR 3.63), DRB1*0404-DQA1*0301-DQB1*0302 (OR 1.59), and DRB1*0801-DQB1*0401-DQB1*0402 (OR 1.25). Preferably, the individual has a haplotype comprising DQB1*02 and DQB1*0302.

In addition, or alternatively, an individual who is genetically predisposed to type 1 diabetes may have first degree relative, i.e. a mother, father, or sibling, who has type 1 diabetes.

Although type 1 diabetes is frequently considered to be a disease that begins in childhood, it can occur at any age. Approximately 50% of individuals develop the disorder before the age 40. An individual who is genetically predisposed to type 1 diabetes may therefore be any age. For example, an individual who is genetically predisposed to type 1 diabetes may be a child. In this case, the individual may be less than 10, less than 9, less than 8, less than 7, less than 6, less than 5, less than 4, less than 3, less than 2, or less than 1 year in age.

The present inventors have discovered that the presence or absence of an exhausted CD8⁺ T cell or lack of CD4⁺ T cell costimulation phenotype in a sample obtained from an individual genetically predisposed to type 1 diabetes, which is indicative of whether the individual is at low risk or high risk of progressing to type 1 diabetes, respectively, can be detected before the individual develops autoantibodies associated with type 1 diabetes. An individual who is genetically predisposed to type 1 diabetes therefore preferably does not have autoantibodies associated with type 1 diabetes. Autoantibodies associated with type 1 diabetes include autoantibodies against islet cells (islet cell antibody; ICA), insulin (insulin autoantibodies; IAA), protein tyrosine phosphatase-related IA-2 protein (islet antigen-2 antibody; IA-2A), glutamic decarboxylase 65 (Glutamic Acid Decarboxylase 65 Autoantibodies; GADA), and/or cation efflux transporter ZnT8 (cation efflux transporter ZnT8 antibody; ZnT8A).

Progression to type 1 diabetes may refer to the development, or onset of, type 1 diabetes. 30

An individual who has, has progressed to, or has developed type 1 diabetes (also referred to as a “progressor”) may show one or more symptoms associated with type 1 diabetes. For example, the individual may have autoantibodies against islet cells (islet cell antibody; ICA), insulin (insulin autoantibodies; IAA), protein tyrosine phosphatase-related IA-2 protein (islet antigen-2 antibody; IA-2A), glutamic decarboxylase 65 (Glutamic Acid Decarboxylase 65 Autoantibodies; GADA), and/or cation efflux transporter ZnT8 (cation efflux transporter ZnT8 antibody; ZnT8A). In addition, or alternatively, the individual may a fasting plasma glucose level of 126 mg/dL (7 mmol/L) or higher, a plasma glucose level of 200 mg/dL (11.1 mmol/L) or higher two hours after administration of a 75g oral glucose load (glucose tolerance test), and/or a glycated hemoglobin level of 6.5 percent or higher.

An individual who does not have, has not progressed to, or has not developed type 1 diabetes (also referred to as a “non-progressor”) may show no symptoms associated with type 1 diabetes. For example, the individual may not have autoantibodies against islet cells (islet cell antibody; ICA), insulin (insulin autoantibodies; IAA), protein tyrosine phosphatase-related IA-2 protein (islet antigen-2 antibody; IA-2A), glutamic decarboxylase 65 (Glutamic Acid Decarboxylase 65 Autoantibodies; GADA), and/or cation efflux transporter ZnT8 (cation efflux transporter ZnT8 antibody; ZnT8A). In addition, or alternatively, the individual may a fasting plasma glucose level of less than 100 mg/dL (5.6 mmol/L), a plasma glucose level of less than 200 mg/dL (11.1 mmol/L) two hours after administration of a 75g oral glucose load (glucose tolerance test), and/or a glycated hemoglobin level of less than 6.5 percent.

As explained above, to determine whether an individual, who is genetically predisposed to type 1 diabetes, has or does not have an exhausted CD8⁺ T cell or lack of CD4⁺ T cell costimulation phenotype, and hence is at low risk or high risk of progressing to type 1 diabetes, respectively, the level of expression of two or more genes selected from the group consisting of KAT2B, CASK, ABCD2, DLG1, SS18, RBL2, RAB7L1, MTHFDI, BMI1, COG5, PDE4D, KERA and VCY in a sample, which may be a sample obtained from the individual, (i.e. the test sample) may be compared with control as explained above..

Where the control is the median expression of two or more genes selected from the group consisting of KAT2B, CASK, ABCD2, DLG1, SS18, RBL2, RAB7L1, MTHFDI, BMI1, COG5, PDE4D, KERA and VCY in a group of individuals comprising (1) individuals who progressed to type 1 diabetes, (2) individuals who did not progress to type 1 diabetes, or (3) comprised both individuals who progressed to type 1 diabetes and individuals who did not progress to type 1 diabetes, the individuals in said group preferably had the same genetic predisposition to type 1 diabetes as the individual from which the test sample was obtained. Most preferably, the individuals in said group had same high risk HLA haplotype, as the individual from which the test sample was obtained. Preferably, the individuals in the group were the same age, as the individual from which the test sample was obtained.

As an alternative, in the case of type 1 diabetes, the control may be a standard curve of expression of two or more genes selected from the group consisting of KAT2B, CASK, ABCD2, DLG1, SS18, RBL2, RAB7L1, MTHFDI, BMI1, COG5, PDE4D, KERA and VCY, derived from samples obtained from a group of individuals who progressed to type 1 diabetes over time. An equal or higher level of expression of genes KAT2B, CASK, ABCD2, DLG1, SS18, RBL2, RAB7L1, MTHFDI, BMI1, COG5, and PDE4D, and an equal or lower level of expression of genes KERA and VCY in a sample obtained from an individual genetically predisposed to type 1 diabetes, compared with the level of expression of these genes shown in the standard curve, at the same time point (age), may indicate that the individual, does not have an exhausted CD8⁺ T cell or lack of CD4⁺ T cell costimulation phenotype, and hence is at high risk of progression to type 1 diabetes. Conversely, a lower level of expression of genes KAT2B, CASK, ABCD2, DLG1, SS18, RBL2, RAB7L1, MTHFDI, BMI1, COG5, and PDE4D, and a higher level of expression of genes KERA and VCY in a sample obtained from an individual genetically predisposed to type 1 diabetes, compared with the level of expression shown in the standard curve at the same time point (age), may indicate that the individual has an exhausted CD8⁺ T cell or lack of CD4⁺ T cell costimulation phenotype, and hence is at low risk of progressing to type 1 diabetes.

Chronic Infection

There are many infectious diseases that are known to cause chronic infections in humans. The causative agents of such diseases include viruses, bacteria and parasites, such as protozoa. Some individuals are capable of clearing chronic infections without treatment, while in others chronic infection progresses. Progression in this context may refer to continuation of the chronic infection, i.e. the individual continues to have the chronic infection, and/or the development of additional disease. For example, in the case of hepatitis C virus (HCV), some individuals develop cirrhosis and/or hepatocellular carcinoma as a result of chronic HCV infection. Predicting the risk of progression in the case of chronic infections has important implications, as treatment of individuals who are at low risk of progression could be avoided. This would be particularly advantageous where treatment is costly or associated with deleterious side effects. Similarly, individuals who are at low risk of progression could be treated, or selected for treatment, for the chronic infection. There thus remains a need in the art for assessing whether an individual with a chronic infection is at high risk or low risk of not progression of said chronic infection.

In addition, although treatments for many chronic infections are known, not all individuals respond to treatment, with the result that some individuals treated experience no, or no significant, benefit as a result of the treatment. This presents a problem, especially where treatment is costly and/or associated with deleterious side effects. There therefore remains a need in the art for methods for determining whether an individual is likely to ultimately respond to a treatment for a chronic infection.

The present inventors have discovered that downregulated expression of genes KAT2B, CASK, ABCD2, DLGI, SS18, RBL2, RAB7L1, MTHFDI, BMII , COG5, and PDE4D, and upregulated expression of genes KERA and VCY, relative to a control indicates that an individual has an exhausted CD8⁺ T cell or lack of CD4⁺ T cell costimulation phenotype. Furthermore, the presence of this phenotype in an individual having a chronic infection, who has been subjected to a treatment for the chronic infection, indicates that the individual is at high risk of not responding to the treatment. It is also expected that the presence of this phenotype in an individual having a chronic infection indicates that the individual is at high risk of progression of the chronic infection.

The present invention thus provides a method of assessing whether an individual with a chronic infection is at high risk or low risk of progression of said chronic infection, as set out in the claims.

As briefly mentioned above, progression in this context may refer to continuation of the chronic infection, i.e. the individual continues to have the chronic infection, a worsening of the chronic infection, such as the development or worsening of one or more clinical symptoms associated with the chronic infection, and/or the development of additional disease resulting from the chronic infection.

In addition, the present invention provides a method of assessing whether an individual with a chronic infection is at high risk or low risk of not responding to a treatment for the chronic infection, wherein the individual has been subjected to the treatment, the method comprising:
(i) determining whether the individual has an exhausted CD8⁺ T cell or lack of CD4⁺ T cell costimulation phenotype using the method of claim 1, wherein the presence of said phenotype indicates that the individual is at high risk of not responding to the treatment, and
wherein the absence of said phenotype indicates that the individual is at low risk of not responding to the treatment.

A method of assessing whether an individual with a chronic infection is at high risk or low risk of not responding to a treatment for the chronic infection, wherein the individual has been subjected to the treatment, by determining whether the individual has or does not have an exhausted CD8⁺ T cell or lack of CD4⁺ T cell costimulation phenotype, the method comprising:
(i) providing a PBMC sample obtained from the individual;
(ii) extracting mRNA from the PBMC sample;

(iii) performing reverse transcription quantitative PCR (RT-qPCR) to convert the mRNA into cDNA and determine the expression level of two more genes selected from the group consisting of: KAT2B, CASK, ABCD2, DLG1, SS18, RBL2, RAB7L1, MTHFDI, KERA, BMI1, COG5, PDE4D, and VCY,
wherein said phenotype is characterised by downregulated expression of genes KAT2B, CASK, ABCD2, DLG1, SS18, RBL2, RAB7L1, MTHFDI, BMI1, COG5, and PDE4D, and upregulated expression of genes KERA and VCY, relative to the level of expression of these genes in an individual who does not have said phenotype, and
wherein the presence of said phenotype indicates that the individual is at high risk of not responding to the treatment, and

wherein the absence of said phenotype indicates that the individual is at low risk of not responding to the treatment, is also provided

Further provided is a risk assessment system to determine the risk of an individual with a chronic infection not responding to a treatment for the chronic infection, wherein the individual has been subjected to the treatment, for use in a method as described herein, the system comprising a tool or tools for determining expression of two more genes selected from the group consisting of: KAT2B, CASK, ABCD2, DLG1, SS18, RBL2, RAB7L1, MTHFDI, KERA, BMI1, COG5, PDE4D, and VCY;
and a computer programmed to compute a risk score of the risk of the individual not responding to the treatment from the gene expression data of the subject.

The method may further comprise (ii) selecting an individual identified as one who is at low risk of not responding to the treatment in step (i) for continued treatment with said treatment; or (ii) subjecting the individual to continued treatment with said treatment if the individual has been identified as one who is at low risk of not responding to the treatment in step (i). Alternatively, the method may comprise, (ii) selecting an individual identified as one who is at high risk of not responding to the treatment in step (i) for treatment; or (ii) subjecting the individual to treatment if the individual has been identified as one who is at high risk of not responding to the treatment in step (i); wherein the treatment comprises inducing a non-exhausted CD8⁺ T cell or CD4⁺ T cell costimulation phenotype in the individual. A non-exhausted CD8⁺ T cell or CD4⁺ T cell costimulation phenotype may be induced in an individual by administering a therapeutically effective amount of an inhibitor of programmed cell death protein 1 (PD-1), e.g. as described herein.

The present invention also provides a method for treating a chronic infection in an individual, wherein the individual has been subjected to a treatment for the chronic infection, the method comprising:
(i) identifying the individual as one who is at low risk of not responding to the treatment using a method as disclosed herein, and
(ii) subjecting the individual to continued treatment with the treatment.

A method for treating a chronic infection in an individual, wherein the individual has been subjected to a treatment for the chronic infection is also provided. This method may comprise:
(i) requesting a test providing the results of an analysis to determine the expression level of two more genes selected from the group consisting of: KAT2B, CASK, ABCD2, DLGI, SSI8, RBL2, RAB7LI, MTHFDI, KERA, BMI1, COG5, PDE4D, and VCY, in a sample obtained from the individual,
wherein downregulated expression of genes KAT2B, CASK, ABCD2, DLGI, SSI8, RBL2, RAB7LI, MTHFDI, BMI1, COG5, and PDE4D, and upregulated expression of genes KERA and VCY, relative to the level of expression of these genes in an individual who does not have an exhausted CD8⁺ T cell or lack of CD4⁺ T cell costimulation phenotype indicates that the individual has said phenotype, and
(ii) subjecting the individual to continued treatment with the treatment if the individual does not have said phenotype.

Further provided is a method for treating a chronic infection in an individual, wherein the individual has been subjected to a treatment for the chronic infection, the method comprising:
(i) identifying the individual as one who is at high risk of not responding to the treatment using a method as disclosed herein, and
(ii) subjecting the individual to treatment if the individual has been identified as one

who is at high risk of not responding to the treatment in step (i),
wherein the treatment comprises inducing a non-exhausted CD8⁺ T cell or CD4⁺ T cell costimulation phenotype in the individual.

A method for treating a chronic infection in an individual, wherein the individual has been subjected to a treatment for the chronic infection, the method comprising:

(i) requesting a test providing the results of an analysis to determine the expression level of two more genes selected from the group consisting of: KAT2B, CASK, ABCD2, DLG1, SS18, RBL2, RAB7L1, MTHFDI, KERA, BMI1, COG5, PDE4D, and VCY,
in a sample obtained from the individual, downregulated expression of genes KAT2B, CASK, ABCD2, DLG1, SS18, RBL2, RAB7L1, MTHFDI, BMI1, COG5, and PDE4D, and upregulated expression of genes KERA and VCY, relative to the level of expression of these genes in an individual who does not have an exhausted CD8⁺ T cell or lack of CD4⁺ T cell costimulation phenotype indicates that the individual has said phenotype, and

(ii) subjecting the individual to continued treatment with the treatment if the individual has said phenotype,
wherein the treatment comprises inducing a non-exhausted CD8⁺ T cell or CD4⁺ T cell costimulation phenotype in the individual, is also provided.

A further embodiment provides a PD-1 inhibitor for use in a method of treating a chronic infection in an individual, wherein the individual has been subjected to a treatment for the chronic infection, the method comprising
(i) determining whether the individual is at high risk of not responding to the treatment using a method as described herein, and

(ii) administering therapeutically effective amount of a PD-1 ligand to the individual if the individual is at high risk not responding to the treatment to induce a non-exhausted CD8⁺ T cell or CD4⁺ T cell costimulation phenotype in the individual.

An individual who is responsive to a treatment (i.e. a responder) may, in response to said treatment, show an improvement in one or more symptoms associated with the chronic infection. For example, in the case of HCV, the level of one or more biomarkers associated with HCV infection, such as HCV RNA levels, as determined e.g. in a PBMC sample isolated from the individual, may be reduced or eliminated in response to the treatment in an individual who is responsive to said treatment. For example, HCV RNA levels may be reduced by >3.5 logᵢₒlU/m1 in an individual who is responsive to the treatment. A individual who is responsive to a treatment may refer to an individual who shows an improvement in one or more symptoms associated by with the chronic infection by the end of said treatment, e.g. when the treatment cycle is complete. Thus, an individual who is responsive to treatment may refer to an individual who will ultimately respond to the treatment.

Similarly, an individual who is not responsive to a treatment (i.e. a non-responder) may, in response to said treatment, show no improvement in one or more symptoms associated with the chronic infection. For example, in the case of HCV, the level of one or more biomarkers associated with HCV infection, such as HCV RNA levels, as determined e.g. in a PBMC sample isolated from the individual, may remain the same or not be significantly reduced in response to the treatment in an individual who is not responsive to said treatment. For example, HCV RNA levels may be reduced by <1.5 logᵢₒlU/m1 in an individual who is not responsive to the treatment. A individual who is not responsive to a treatment may refer to an individual who shows no improvement in one or more symptoms associated with the chronic infection by the end of said treatment, e.g. when the treatment cycle is complete. Thus, an individual who is not responsive to treatment may refer to an individual who will not ultimately respond to the treatment.

As is the case with most treatments, response to treatment may not be absolute. For example, where the individual has a chronic HCV infection, an individual who is at low risk of not respond to a treatment may have a 80% or greater probability of responding to the treatment and an individual who is at high risk of not responding to treatment may have a 54% or lower probability of responding to the treatment. In the case of chronic HCV infection, the treatment may be treatment with ribavirin and pegylated interferon-alpha.

A chronic infection, as referred to herein, may be a chronic viral infection, a chronic bacterial infection or a chronic parasitic infection. The chronic infection may be chronic hepatitis C (HCV) or chronic Hepatitis B (HBV) infection.

Vaccination

For any given vaccine there are some individuals who do not respond to said vaccine, i.e. mount an effective immune response to the vaccine. Identifying individuals who are at high risk of not responding to a vaccine is important as it allows, for example, such individuals to be subjected to booster vaccination, or monitored for subsequent infection and treated where necessary. There therefore remains a need in the art for methods for determining whether an individual is at high risk of not responding to a vaccine.

The present inventors have discovered that downregulated expression of genes KAT2B, CASK, ABCD2, DLGI, SS18, RBL2, RAB7L1, MTHFDI, BMII , COG5, and PDE4D, and upregulated expression of genes KERA and VCY, relative to a control indicates that an individual has an exhausted CD8⁺ T cell or lack of CD4⁺ T cell costimulation phenotype. Furthermore, the presence of this phenotype in an individual who has received a vaccine indicates that the individual is at high risk of not mounting an effective immune response to the vaccine.

An individual who has mounted an effective immune response to a vaccine may have antibodies against said vaccine. Antibodies against the vaccine in the individual may, for example, be increase relative to a baseline. Methods for measuring antibodies to a particular vaccine are known in the art and include ELISA, for example. In the case of an influenza vaccine, antibodies against the vaccine may be measured using a haemagglutination-inhibition assay. Alternatively, an individual who has mounted an effective immune response to a vaccine may have complete or partial protection from the disease against which the vaccine was directed. For example, an individual who has mounted an effective immune response to an influenza A vaccination may have complete or partial protection from subsequent influenza caused by a strain against which the vaccine was directed.

Similarly, an individual who has not mounted an effective immune response to a vaccine may not have antibodies against said vaccine, or may not have protection from the disease against which the vaccine was directed. For example, an individual who has not mounted an effective immune response to an influenza vaccine may not have protection from influenza caused by the strain against which the vaccine was directed.

An individual who has been identified as being at high risk of not mounting an effective immune response to a vaccine against a disease may be subjected to vaccination with, or selected for vaccination with, a further dose of the same vaccine, ora different vaccine against the same disease. A further dose of the vaccine may be identical to a first dose administered to the individual or may be different. For example, the further dose may be an increased dose compared with a first dose administered. Where the individual is subjected to vaccination with, or selected for vaccination with, a different vaccine against the same disease, said vaccine may be capable of eliciting an immune response to a different disease-associated antigen compared with a first vaccine administered to the individual.

Alternatively, or additionally, the vaccine may comprise a different adjuvant and/or increased amount of adjuvant, compared with a first vaccine or first vaccine dose administered to the individual.

Alternatively, an individual who has been identified as being at high risk of not mounting an effective immune response to a vaccine against a disease may be subjected to a prophylactic treatment for the disease against which the vaccine was directed, or selected for treatment with such a prophylactic treatment. A prophylactic treatment may refer to a preventive treatment. For example, an individual who has been identified as being at high risk of not mounting an effective immune response to a malaria vaccine may be subjected to treatment with an antimalarial or selected for treatment with an antimalarial. Prophylactic and preventive treatments for many diseases are known in the art but may be less-preferred than vaccination due to e.g. side effects, in the case of certain types of antimalarial medication.

As further alternative, an individual who has been identified as being at high risk of not mounting an effective immune response to a vaccine may be subjected to passive vaccination for the disease against which the vaccine was directed, or selected for treatment with such a passive vaccine. Passive vaccination involves the transfer of antibodies against the disease in question to an individual in need thereof. The antibodies may be derived from donor individuals or produced in vitro, such as monoclonal antibodies. Immunity derived from passive vaccination usually lasts a few weeks or months and it thus is generally less preferred than “active” vaccination but may be useful where there is a high risk that the individual will not mounting an effective immune response to a vaccine which has been administered.

A vaccine, as referred to herein, may be a vaccine for a viral, bacterial or parasitic infection. Parasitic infections, include protozoal infections, such as malaria. The vaccine may be a vaccine against influenza virus, in particular influenza A virus, yellow fever virus, or malaria.

Infection-Associated Immunopathology

Some diseases give rise to an excessive inflammatory response in some individuals. For example, infection with dengue virus can result in a wide range of clinical manifestations ranging from asymptomatic infection or self-limiting fever (uncomplicated dengue) to hemorrhagic fever. It is thought that hemorrhagic fever may caused by an excessive inflammatory response to the virus in the individual. Other disease in which an excessive immune response is thought to result in a more severe disease, include influenza virus and Sars coronavirus infections.

The present inventors have discovered that upregulated expression of genes KAT2B, CASK, ABCD2, DLG1, SS18, RBL2, RAB7L1, MTHFD1, BMI1, COG5, and PDE4D, and downregulated expression of genes KERA and VCY, relative to a control indicates that an individual does not have an exhausted CD8⁺ T cell or lack of CD4⁺ T cell costimulation phenotype. The absence of this phenotype in an individual indicates that the individual, in particular an individual suffering from an infection, is at high risk of infection-associated immunopathology.

The infection-associated immunopathology, as referred to herein, may be any infection in which an individual's immune response to the infection results in tissue damage in the individual. Tissue damage may be manifested as clinical pathology (see for, example, Rouse et al. Nat Rev Immunol 2010;10:514-26). Many infections causing immunopathology are known in the art. In one example, the infection-associated immunopathology may be the result of dengue haemorrhagic fever. Alternatively, the infection-associated immunopathology may be the result of influenza virus infection (in particular influenza A virus infection), cytomegalovirus (CMV) infection, SARS, Epstein-Barr virus (EBV) infection, Hepatitis A, B, C or E virus infection, coxsackie virus infection, or chikungunya virus infection.

Transplantation

Following transplantation, individuals may experience acute rejection, chronic rejection, humoral rejection, or cellular rejection of the transplant. Acute transplant rejection occurs over a period of a few days. Chronic rejection occurs weeks or months after the transplant. Chronic rejection is the most common form of transplant rejection. Given the deleterious effect of transplant rejection, as well as the costs involved, there remains a need in the art for predicting whether an individual is at high or low risk of transplant rejection. Predicting the risk of transplant rejection may also allow for treatment of high risk individuals prior to or following transplantation to reduce the risk of transplant rejection. Transplantation, as referred to herein, is preferably allograft transplantation. Rejection thus preferably refers to allograft rejection.

The present inventors have discovered that upregulated expression of genes KAT2B, CASK, ABCD2, DLG1, SS18, RBL2, RAB7L1, MTHFD1, BMI1, COG5, and PDE4D, and downregulated expression of genes KERA and VCY, relative to a control indicates that an individual does not have an exhausted CD8⁺ T cell or lack of CD4⁺ T cell costimulation phenotype. It is expected that the absence of this phenotype indicates that the individual is at high risk of transplant rejection, in particular acute transplant rejection. An exhausted CD8⁺ T cell phenotype is characterised by a reduced proliferative response and impaired cytokine production. This state, and/or the associated state of limited CD4 costimulation, facilitates tolerance of transplanted allografts in the same manner that an exhausted antiviral T cell response facilitates persistence of the pathogen (Thorp et al., Curr Op Org Transplant 2015;20(1):37-42). By measuring the presence or extent of an exhausted CD8⁺ T cell phenotype, the risk of reaching the clinical endpoint of acute (Steger et al. Transplantation 2008;85(9):1339) or chronic (Sarraj et al. PNAS 2014;111(33):12145-50) allograft rejection can be determined. An individual at high risk of transplant rejection may be subjected to more frequent and/or more intense monitoring than is usual following transplantation, such that, for example, indications of transplant rejection can be detected and treated earlier when they are more responsive.

Cancer

In the case of cancer, progression of the disease differs between different individuals. In some individuals the disease progresses quickly, while in others progression is slow. Given the deleterious side effects of many cancer treatments, as well as the costs involved, there remains a need in the art for predicting whether an individual is at high or low risk of cancer progression.

The present inventors have discovered that downregulated expression of genes KAT2B, CASK, ABCD2, DLGI, SS18, RBL2, RAB7L1, MTHFDI, BMII , COG5, and PDE4D, and upregulated expression of genes KERA and VCY, relative to a control indicates that an individual has an exhausted CD8⁺ T cell or lack of CD4⁺ T cell costimulation phenotype. Furthermore, it is expected that the presence of this phenotype in an individual indicates that the individual is at high risk of cancer progression.

As mentioned above, assessing whether an individual is at high risk or low risk of cancer progression may be useful in the context of cancer treatment by allowing patients who are likely to benefit from a given treatment to be identified. For example, a given cancer treatment may not show benefit in all patients with a particular cancer but may show benefit in patients at high risk of cancer progression. In addition, or alternatively, a cancer treatment may be associated with side-effects which are too severe for use of the treatment in all patients with a particular cancer but may be acceptable as a treatment for individuals at high risk of cancer progression. Some cancer treatments may similarly be too costly to administer to all patients with a particular cancer but may be justified for treatment of patients at high risk of cancer progression.

Methods for assessing whether an individual is at high risk or low risk of cancer progression may also be useful in the context of clinical trials as patients at high risk of cancer expression are expected to reach a relevant trial endpoints more quickly or frequently, with the result that a clinical trial involving only individuals assessed to be at high risk of cancer progression will need to include fewer individuals resulting in cost saving, as well as increasing the likelihood of detecting beneficial effect(s) of the treatment being trialled.

Cancer progression may refer to an increase in the size and/or number of tumours, an increase in organ dysfunction, e.g. as a result of neoplastic infiltration, the emergence or an increase in the number of tumour metastases, a change in the stage or grade of the malignancy, and/or the recurrence of a malignancy after a period of remission, in the individual.

In Vitro Methods

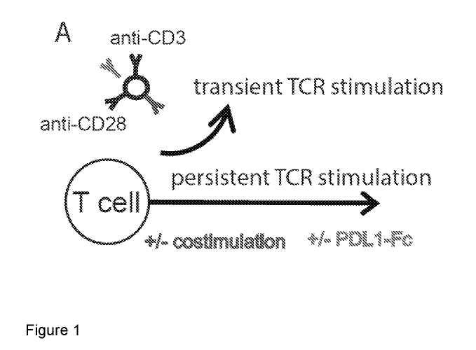

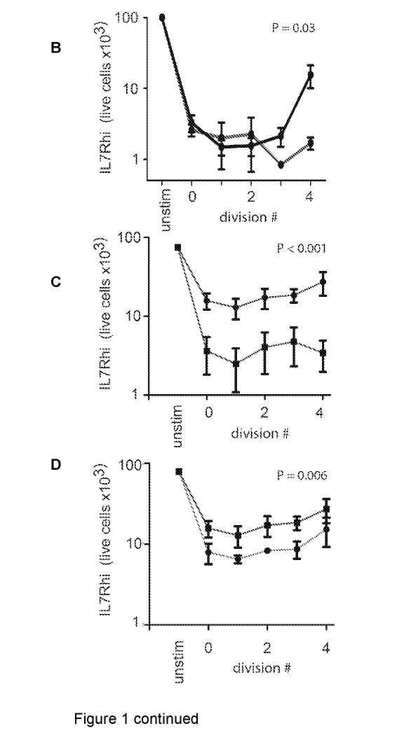

To date, it has not been possible to reproduce the phenotype of CD8⁺ T cell exhaustion in primary human CD8⁺ T cells using in vitro cell culture. The present inventors have discovered that the use of anti-CD2 mediated costimulation in addition to anti-CD3/anti-CD28 mediated T cell activation (FIG. 1) specifically prevents the development of an exhausted CD8⁺ T cell phenotype when compared to the use of anti-CD3/anti-CD28 mediated T cell activation alone (FIG. 1 C-D). In this context, CD2-mediated costimulation reproduces the transcriptional signature of CD8⁺ T cell exhaustion seen, for example, in autoimmune disease and in chronic infection. However, for purposes of an in vitro assay, it is preferable to measure surrogate markers of a CD8⁺ T cell exhaustion phenotype, such as IL7R and PD-1, during T cell proliferation. An exhausted CD8⁺ T cell phenotype is characterised by low expression of IL7R and high expression of PD-1, e.g. relative to the level of expression of these genes in an individual who does not have said phenotype, while a non-exhausted CD8+ T cell phenotype is characterised by high expression of IL7R and low expression of PD-1, e.g. relative to the level of expression of these genes in an individual who does not have said phenotype. Expression of IL7R and PD-1 can be determined by any method known in the art or described herein, such as multi-parameter flow cytometry. Hence, primary human CD8+T cells from an individual can be induced to differentiate into CD8+T cells with an exhausted CD8⁺ T cell phenotype (using anti-CD3/28 antibodies) or into CD8⁺T cells with a non-exhausted CD8⁺ T cell (using anti-CD2/3/28 antibodies). Such CD8⁺ T cells, as well as the methods to generate such CD8⁺T cells, may find application in different fields, including those described herein.

Thus, the present invention provides a method of preparing CD8⁺T cells with a non-exhausted CD8⁺ T cell phenotype, the method comprising:
(i) providing a sample of CD8⁺ T cells obtained from an individual;
(ii) incubating the CD8⁺ T cells in the presence of anti-CD2, anti-CD3 and anti-CD28 antibodies, and IL2; and
(iii) determining the expression level of IL7R and PD-1 by the CD8⁺ T cells; wherein a higher expression of IL7R and a lower expression of PD-1 by the CD8⁺T cells following incubation in the presence of the anti-CD2, anti-CD3 and anti-CD28 antibodies, and IL2 compared with prior to incubation, indicates that the CD8⁺ T cells have a non-exhausted CD8⁺ T cell phenotype.

The present invention also provides a method of preparing CD8⁺T cells with an exhausted CD8⁺ T cell phenotype, the method comprising:
(i) providing a sample of CD8⁺ T cells obtained from an individual;
(ii) incubating the CD8⁺ T cells in the presence of anti-CD3 and anti-CD28 antibodies, and IL2; and
(iii) determining the expression level of IL7R and PD-1 CD8⁺ T cells; wherein a lower expression of IL7R and a higher expression of PD-1 by the CD8⁺T cells following incubation in the presence of the anti-CD3 and anti-CD28 antibodies, and IL2 compared with prior to incubation, indicates that the CD8⁺ T cells have an exhausted CD8⁺ T cell phenotype. The method may further comprise incubating the CD8⁺ T cells in the presence of PDL1, such as an Fc-chimaeric PDL1 protein.

The method may further comprise administering the CD8⁺ T cells to the individual from which the CD8⁺ T cells were obtained.

Adoptive cellular therapy is a method which involves the isolation and transfer of autologous, ex-vivo conditioned immune cells with the aim of modulating an endogenous immune response. Adoptive transfer of activated effector cells has been used with success in cancer (Rosenberg et al. Nat Rev Cancer 2008;8(4):299-308) and chronic infection (Moss et al. Nat Rev Immunol 2005;5:9-20) while transfer of thimaeric' T cells specifically transduced with antigen-receptors specific for tumour components (CARs) have also shown promise (Porter et al. NEJM 2011;365:725-33). Similarly, in autoimmune disease, infection-associated immunopathology, and transplantation, adoptive transfer of T cells with a regulatory phenotype has shown promise in mediating immunoregulation and resolution of disease or organ dysfunction (Riley, J L. Immunity 2009;30(5):656-65). However, in each instance of adoptive cellular therapy it is essential that the cellular phenotype induced by ex-vivo conditioning creates is characterized by the ability to perform effector/regulatory function and to persist long-term in vivo (Riddell et al. Ann Rev Immunol 1995;13:545-86). As a T cell undergoes effector differentiation there is a progressive loss of its ability to persist and to carry out its intended in vivo function after adoptive transfer (Gattinoni et al. Nat Rev Immunol 2006;6:383). Current methods of in vitro differentiation of T cells result in the development of effector function but loss of longevity (Gattinoni et al. Nat Rev Immunol 2006;6:383). Some methods aim to prevent this endpoint and are being further trialed (Gattinoni et al. Nat Med 2011;17(10):1290-7). It has been proposed that treatments limiting the development of CD8⁺T cell exhaustion may maximize the longevity and success of adoptive therapy (Kamphorst A O, Immunotherapy 2013;5(9):975-87) but there has to date been no means by which exhaustion in primary human T cells can be prevented during ex-vivo conditioning. We propose that by providing additional CD2-mediated costimulation during ex-vivo conditioning exhaustion may be usefully prevented prior to adoptive transfer.

Thus, the present invention provides a method of preparing CD8+ T cells with a non-exhausted CD8⁺ T cell phenotype for autologous cellular therapy, the method comprising:
(i) providing a sample of CD8⁺ T cells obtained from an individual;
(ii) incubating the CD8⁺ T cells in the presence of an anti-CD2 antibody; and
(iii) determining the expression level of IL7R and PD-1 by the CD8⁺ T cells;
wherein a higher expression of IL7R and a lower expression of PD-1 by the CD8⁺T cells following incubation in the presence of the anti-CD2 antibody compared with prior to incubation, indicates that the CD8⁺ T cells have a non-exhausted CD8⁺ T cell phenotype. The method may further comprise administering the CD8⁺ T cells to the individual from which the CD8⁺ T cells were obtained.

The correlation of CD8⁺ T-cell exhaustion with disease outcome has obvious therapeutic implications. The present inventors have shown using an in vitro model that the use of biologic agents to alter CD8⁺ T-cell co-stimulation can modify CD8⁺ T cell exhaustion. CD8⁺ T cell exhaustion may be promoted by enhancing costimulation (using an anti-CD2 antibody) or limited by providing additional coinhibitory signals (by using e.g. an Fc-chimaeric PDL1 protein). The in vitro assay may be used for screening a compound libraries and/or additional coinhibitory or costimulatory molecules for their potential effects on T cell exhaustion. This would facilitate selection of a substance capable of inducing an exhausted CD8⁺ T cell phenotype, or a non-exhausted CD8⁺ T cell phenotype, in an individual in need thereof, as described elsewhere herein. For example, a substance capable of inducing an exhausted CD8⁺ T cell phenotype may be used in the treatment of autoimmune diseases.

Thus, provides is an in vitro method for identifying a substance capable of inducing an exhausted CD8⁺ T cell phenotype in an individual, the method comprising:
(i) providing a sample of CD8⁺ T cells;
(ii) incubating the CD8⁺ T cells in the presence of anti-CD2, anti-CD3 and anti-CD28 antibodies, IL2, and in the presence or absence of a substance of interest; and
(iii)determining the expression level of IL7R and PD-1 by the CD8⁺ T cells; wherein a lower expression of IL7R and a higher expression of PD-1 by the CD8⁺T cells in the presence of the substance of interest than in the absence of the substance of interest indicates that the substance is capable of inducing an exhausted CD8⁺ T cell phenotype in an individual.

Also provided is an in vitro method for identifying a substance capable of inducing a non-exhausted CD8⁺ T cell phenotype in an individual, the method comprising:
(i) providing a sample of CD8⁺ T cells;
(ii) incubating the CD8⁺ T cells in the presence of anti-CD3 and anti-CD28 antibodies, IL2, and in the presence or absence of a substance of interest; and
(iii)determining the expression level of IL7R and PD-1 by the CD8⁺ T cells; wherein a higher expression of IL7Rand a lower expression of PD-1 by the CD8+T cells in the presence of the substance of interest than in the absence of the substance of interest indicates that the substance is capable of inducing a non-exhausted CD8⁺ T cell phenotype in an individual. The method may further comprise incubating the CD8⁺ T cells in the presence of PDL1, such as an Fc-chimaeric PDL1 protein.

Where a method comprises determining the expression level of IL7R and PD-1 by the CD8⁺ T cells, the method may further comprise measuring/determining cell proliferation. Methods for measuring/determining cell proliferation are known in the art and include e.g. CFSE dilution.

The method may also comprise formulating a substance identified as capable of inducing an exhausted CD8⁺ T cell phenotype in an individual, or capable of inducing a non-exhausted CD8⁺ T cell phenotype in an individual, into a medicament. Formulation into a medicament may comprise formulating the substance with a suitable pharmaceutical excipient. Suitable excipients are known in the art.

Treatment

In the case of autoimmune disease, chronic infection, infection-associated immunopathology and cancer, treatment may refer to therapeutic treatment of ongoing disease intended to manage the disease, treatment to cure the disease, or treatment to provide relief from the symptoms of the disease, as well as prophylactic treatment to prevent disease in an individual at high risk of developing a disease, as applicable.

In the case of autoimmune disease, chronic infection, infection-associated immunopathology and cancer, treatment may be any known treatment for the disease in question. The application of such known treatments is well within the capabilities of the skilled practitioner.

Treatment may comprise inducing an exhausted CD8⁺ T cell or lack of CD4⁺ T cell costimulation phenotype, or inducing a non-exhausted CD8⁺ T cell or CD4⁺ T cell costimulation phenotype, in the individual, as applicable in the context. An exhausted CD8⁺ T cell or lack of CD4⁺ T cell costimulation phenotype may be induced in an individual by administering a therapeutically effective amount of a programmed cell death protein 1 (PD-1) ligand, such as programmed death-ligand 1 (PDL-1). A non-exhausted CD8⁺ T cell or CD4⁺ T cell costimulation phenotype may be induced in an individual by administering a therapeutically effective amount of an inhibitor of PD-1. PD-1 inhibitors are known in the art and include nivolumab (PD-1 blockade). Alternatively, a non-exhausted CD8⁺ T cell or CD4⁺ T cell costimulation phenotype may be induced in an individual by administering a therapeutically effective amount of an inhibitor of cytotoxic T-lymphocyte-associated protein 4 (CTLA4). Again, inhibitors of CTLA4 are known in the art and include ipilimumab. Such ‘checkpoint’ blockade of exhaustion-associated inhibitory receptors has proved a successful therapy for some cancer patients (Pardoll, DM. The blockade of immune checkpoints in cancer immunotherapy. Nat Rev Cancer 2012;12(4):252-64). However, only a minority of patients show a sustained response to this promising therapy. A major focus remains the identification of markers allowing prediction of response to checkpoint therapy and, while some progress has been made (Day et al., Nature 2006; Erbst et al., Nature 2014), no biomarker is sufficiently robust to allow clinical stratification and patient selection. Detection of a CD8⁺T cell exhaustion or lack of CD4⁺ T cell costimulation phenotype is expected to allow individuals most likely to benefit from such therapy to be identified.

Alternatively, treatment may comprise treatment with CD8⁺ T cells having an exhausted, or non-exhausted, CD8⁺ T cell phenotype, as applicable in the context. CD8⁺T cells with an non-exhausted CD8⁺ T cell phenotype are expected to be useful in the treatment of diseases in which a non-exhausted CD8⁺ T cell phenotype is beneficial, such as cancer treatment, for example, while CD8⁺T cells with an exhausted CD8⁺ T cell phenotype are expected to be useful in the treatment of disease in which an exhausted CD8⁺ T cell phenotype is beneficial, such as treatment of autoimmune diseases. Accordingly, an individual at high risk of cancer progression may be treated with CD8⁺ T cells which have a non-exhausted CD8⁺ T cell phenotype, for example. Methods for preparing CD8⁺ T cells having an exhausted, or non-exhausted, CD8⁺ T cell phenotype are described herein. In the context of treatment, the CD8⁺ T cells are preferably CD8⁺ T cells obtained from the individual to be treated which have been induced to exhibit an exhausted, or non-exhausted, CD8⁺ T cell phenotype as required by the context.

Thus, also provided is a plurality of CD8⁺ T cells with a non-exhausted CD8⁺ T cell phenotype for use in a method of treatment in an individual, the method comprising:
(i) providing a sample of CD8⁺ T cells obtained from the individual;
(ii) incubating the CD8⁺ T cells in the presence of anti-CD2, anti-CD3 and anti-CD28 antibodies, and IL2;
(iii) determining the expression level of IL7R and PD-1 by the CD8⁺ T cells;
wherein a higher expression of IL7R and a lower expression of PD-1 by the CD8⁺T cells following incubation in the presence of the anti-CD2, anti-CD3 and anti-CD28 antibodies, and IL2, compared with prior to incubation, indicates that the CD8⁺ T cells have a non-exhausted CD8⁺ T cell phenotype; and

administering the CD8⁺ T cells having a non-exhausted CD8⁺ T cell phenotype to the individual.

Further provided is a plurality of CD8⁺T cells with an exhausted CD8⁺ T cell phenotype for use in a method of treatment in an individual, the method comprising:
(i) providing a sample of CD8⁺ T cells obtained from the individual;
(ii) incubating the CD8⁺ T cells in the presence of anti-CD3 and anti-CD28 antibodies, and IL2;
(iii)determining the expression level of IL7R and PD-1 CD8⁺ T cells; wherein a lower expression of IL7R and a higher expression of PD-1 by the CD8+T cells following incubation in the presence of the anti-CD3 and anti-CD28 antibodies, and IL2, compared with prior to incubation, indicates that the CD8⁺ T cells have an exhausted CD8⁺ T cell phenotype; and
administering the CD8⁺ T cells having an exhausted CD8⁺ T cell phenotype to the individual.

In the case of autoimmune disease, treatment may comprise selecting for treatment, or treating, an individual identified as one who is at high risk of autoimmune disease progression with a more frequent or more intense disease treatment regimen, or with a disease regimen not normally administered during the maintenance phase of the autoimmune disease. A more frequent or more intense disease treatment regimen may refer to a disease treatment regimen that is more frequent or more intense than the treatment normally administered during the maintenance phase of the autoimmune disease. An example of a more intense disease treatment regimen is intermittent rituximab treatment, e.g. in the case of AAV. Similarly treatment in this context may comprise selecting for treatment, or treating, an identified as one who is at low risk of autoimmune disease progression with a less frequent or less intense disease treatment regimen, or with a disease regimen not normally administered during the maintenance phase of the autoimmune disease. A less frequent or less intense disease treatment regimen may refer to a disease treatment regimen that is less frequent or less intense than the treatment normally administered during the maintenance phase of the autoimmune disease. For example, “treatment” with a less frequent or less intense disease treatment regimen may comprise stopping maintenance therapy for a subject identified as having a low risk phenotype. Alternatively, an individual who has been identified as one who is at high risk of autoimmune disease progression may be selected for treatment, or treated with a prophylactic treatment for the autoimmune disease in question. For example, where an individual is identified as one who is at high risk of progressing to type 1 diabetes the individual may be selected for, and/or subjected to, treatment for type 1 diabetes, such as an early stage treatment or a prophylactic treatment. A prophylactic treatment may refer to a preventive treatment. Individuals identified as being at high risk of IPF progression may be treated, or selected for treatment, with nintedanib, pirfenidone, or a phosphodiesterase inhibitor (e.g. sildafenil). Alternatively, individuals identified as being at high risk of IPF progression may be treated, or selected for treatment, with immunosuppressive therapy, such as treatment with azathioprine, colchicine, cyclophosphamide, cyclosporine, endothelin receptor antagonists, anti-TNF therapy, methotrexate, Interferon gamma-1b or penicillamine. Immunosuppressive therapy is not normally employed as a treatment in IPF but may be beneficial in individuals who are at high risk of IPF progression. As a further alternative, treatment of individuals identified as being at high risk of IPF progression may comprise increased levels of supportive care, such as monitoring, investigation, supplemental oxygen therapy, pulmonary rehabilitation, anticoagulation, or prophylactic vaccination.

In the case of infection-associated immunopathology, treatment is particularly challenging as it requires the need to balance pathogen-directed immunity, and immunopathology driven by an aggressive immune response, in the individual. Identifying individuals at high risk of infection-associated immunopathology is therefore important, as it allows treatment to be targeted at those most likely to require it without unnecessarily suppressing the immune response in individuals at low risk of infection-associated immunopathology. For example, an individual who is at high risk infection-associated immunopathology may be treated, or selected for treatment, with an immunomodulatory treatment, such as corticoid steroid therapy. Corticoid steroid therapy has been trialled, for example, in the treatment of immunopathology associated with SARS coronavirus infection (Lee et al., NEJM 2003;348:1986-94) and pneumococcal infection (Damjanovic, D et al. Marked improvement of severe lung immunopathology by influenza-associated pneumococcal superinfection requires both the control of bacterial superinfection and host immune responses. Am J Pathol 2013;183(3):868-80).

In the case of chronic HCV infection, treatment may be treatment with ribavirin and pegylated interferon-alpha ora direct-acting anti-viral agent (Liang et al. NEJM 2014, 370:2043-7).

In the case of transplant rejection, an individual determined to be at high risk of transplant rejection may be treated, or selected for treatment, with a different or more intense immunosuppressive therapy than that normally administered following transplantation.

Experimental Section

Materials and Methods

Patients.

Ethical approval for this study was obtained from the Cambridge Local Research Ethics Committee (REC reference numbers 04/023, 08/H0306/21,08/H0308/176) and informed consent was obtained from all subjects enrolled.

AAV patients

59 AAV patients attending or referred to the specialist vasculitis unit at Addenbrooke's hospital, Cambridge, UK between July 2004 and May 2008 were enrolled into the present study. Active disease at presentation was defined by Birmingham vasculitis activity score (BVAS³¹) and the clinical impression that induction immunosuppression would be required. Prospective disease monitoring was undertaken monthly with serial BVAS disease scoring³¹ and full biochemical, hematological and immunological profiling followed by treatment with an immunosuppressant and tapering dose steroid therapy. At each time-point of follow-up, disease activity was allocated into one of three categories defined as follows:

1. Flare (at least 1 major or 3 minor BVAS criteria),

2. low grade activity (0 major and 1-2 minor BVAS criteria),

3. no activity (0 major or minor BVAS criteria). All disease flares were crosschecked against patient records to confirm clinical impression of disease activity and the need for intensified therapy as a result. Disease activity scoring was performed by a single investigator (EFM), blinded to gene expression data at the time of scoring. Additional flares were defined in the absence of BVAS scoring if patients attended for emergency investigation (bronchoscopy, or specialist ophthalmological or Ear/Nose/Throat surgical review) that confirmed evidence of active disease. To differentiate between discrete flares, clear improvement in disease activity was required in the form of an improvement in flare-related symptoms together with a reduction in BVAS score, a reduction in markers of inflammation (CRP, ESR), and a reduction in immunosuppressive therapy.

SLE Patients

The SLE cohort was composed of 23 patients attending or referred to the Addenbrooke's Hospital specialist vasculitis unit between July 2004 and May 2008 meeting at least four ACR SLE criteria³², presenting with active disease (defined below) and in whom immunosuppressive therapy was to be commenced or increased. Following treatment with an immunosuppressant patients were followed up monthly. Disease monitoring was undertaken with serial BILAG disease scoring³³ and full biochemical, hematological and immunological profiling.

A discrete disease flare required all three of the following prospectively defined criteria:

1. new BILAG score A or B in any system

2. clinical impression of active disease by the reviewing physician

3. the intention to increase in immunosuppressive therapy as a result.

Additional flares were defined in the absence of BILAG scoring if patients were admitted directly to hospital as emergency cases for increased immunosuppressive therapy. To differentiate between disease flares clear improvement in disease activity was required in the form of diminished flare-related symptoms together with a reduction in both BILAG score and immunosuppressive therapy.

IBD Patients

Patients with active CD and UC were recruited from a specialist IBD clinic at Addenbrooke's Hospital, prior to commencing treatment. Diagnosis was made using standard endoscopic, histologic, and radiological criteria³⁴. Patients who had already received immunomodulators or corticosteroids were excluded. Enrolled patients were managed conventionally using a step-up strategy3.

Assessment of disease activity was in accordance with national and international guidelines and included consideration of symptoms, clinical signs, and objective measures, including blood tests (C-reactive protein [CRP], erythrocyte sedimentation rate [ESR], hemoglobin concentration, and serum albumin), stool markers (calprotectin), and mucosal assessment (by sigmoidoscopy or colonoscopy) where appropriate. Validated scoring tools were used as another means of assessing disease activity (Harvey-Bradshaw severity index³⁵ or simple clinical colitis activity index³⁶ for CD and UC, respectively), although these were not used to guide treatment decisions. All clinicians were blinded to the microarray results.

For each disease, all patients were not included in all analyses as, for example, comparison of modular network analysis in related cell types required that samples passing QC filtering were available for all cell types for all patients. Our previous publications have shown that the sample sizes used here are adequate to detect reproducible signatures correlating with clinical traits.

Follow up Analysis.

Comparisons of outcome and associated clinical variables between subgroups were analyzed using the Kaplan-Meier log-rank test and non-parametric Mann Whitney U test or the Chi-square test as appropriate. Correction for multiple testing was applied using the Bonferroni method or false discovery rate (FDR, Benjamini and Hochberg method) where appropriate as indicated.

Cell Separation and RNA Extraction.

Venepuncture was performed at a similar time of day in all cases to minimize gene expression differences arising from circadian variation³⁷. Peripheral blood mononuclear cells (PBMC), CD4 and CD8 T cells were isolated from 110m1 of whole blood by centrifugation over ficoll and positive selection using magnetic beads as previously described²⁰. The purity of separated cell subsets was determined by flow cytometry and included as a covariate in downstream correlation and network analyses. Total RNA was extracted from each cell population using an RNeasy mini kit (Qiagen) with quality assessed using an Agilent BioAnalyser 2100 and RNA quantification performed using a NanoDrop ND-1000 spectrophotometer.

Microarray Gene Expression Profiling

HsMediante25k custom spotted microarray

Total RNA (250 ng) was converted into double-stranded cDNA and labelled with Cy3- or Cy5-dCTP as previously described²⁰. Appropriate Cy3- and Cy5-labelled samples were pooled and hybridized to custom spotted oligonucleotide microarrays (HsMediante25k) comprised of probes representing 25,342 genes and control features³⁸. All samples were hybridized in duplicate, using a dye-swap strategy, against a common reference RNA derived from pooled PBMC samples. Following hybridization, arrays were washed and scanned on an Agilent G2565B scanner.

Affymetrix Human Gene 1.0 ST Microarray

Aliquots of total RNA (200 ng) were labeled using Ambion WT sense Target labeling kit and hybridized to Human Gene 1.0 or 1.1 ST Arrays (Affymetrix) as described. After washing, arrays were scanned using a GS 3000 or Gene Titan scanner (Affymetrix) as appropriate.

Published Datasets

Published datasets were accessed through either NCBI-GEO or ArrayExpress, imported into R using the Bioconductor package GEOquery and analyzed as described. Search criteria incorporated the name of individual diseases and were filtered to human datasets but not by platform used. Studies were only included if they met the following criteria:

1. Similar QC filters as applied to the data produced in-house were satisfied (described below).

2. Samples were taken at an analogous time-point to those from which the costimulation and exhaustion signatures in autoimmunity were identified. i.e. samples taken during active disease without concurrent immunosuppressive therapy.

3. Clinical outcome data was available.

It was not feasible to build a unified predictive model across all available datasets as they originated from different groups and were performed on mutually incompatible microarray platforms.

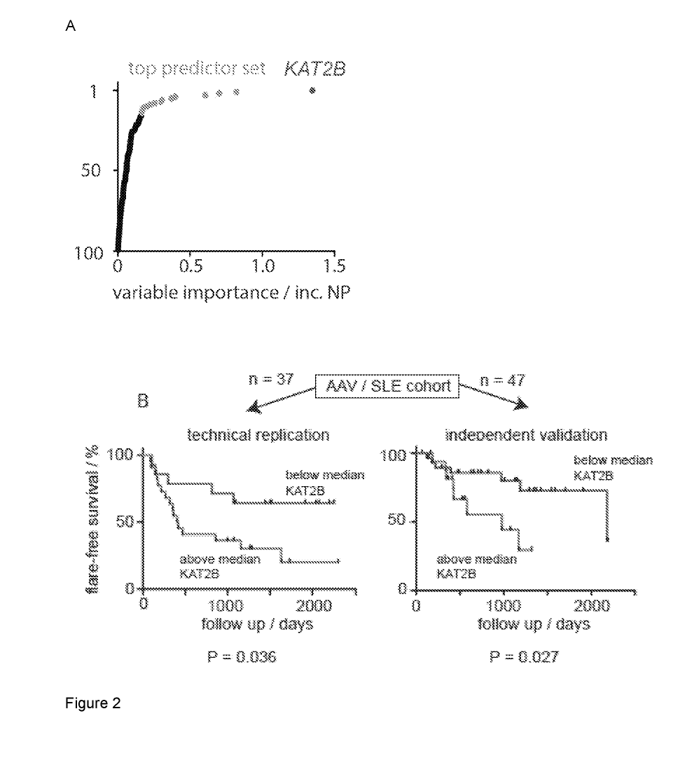

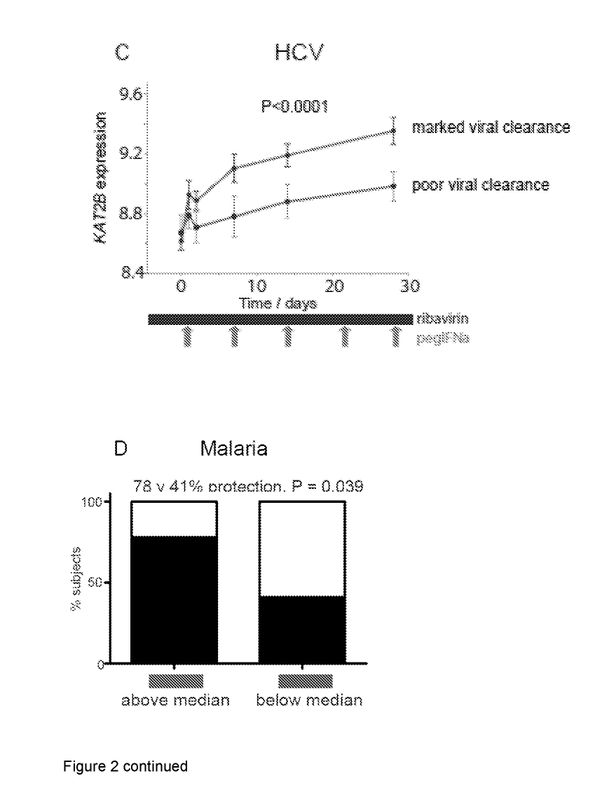

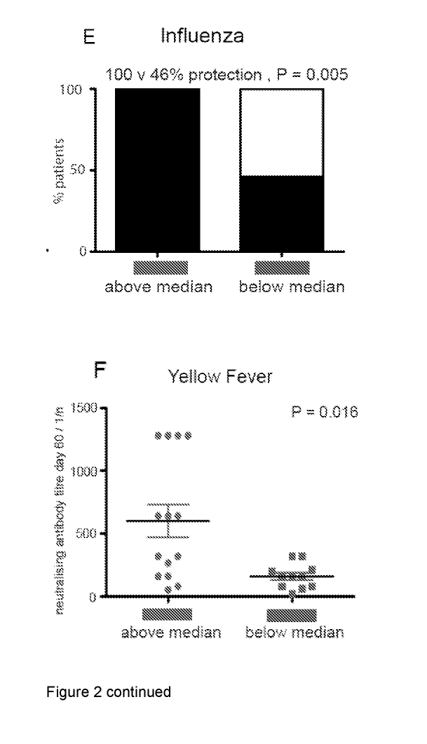

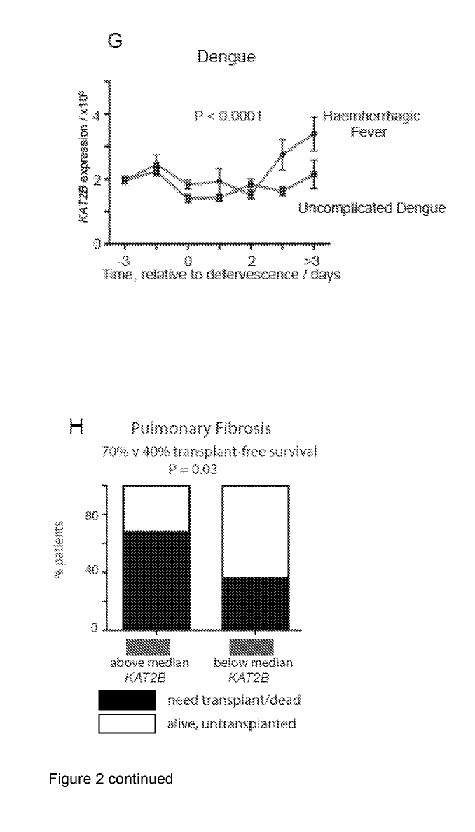

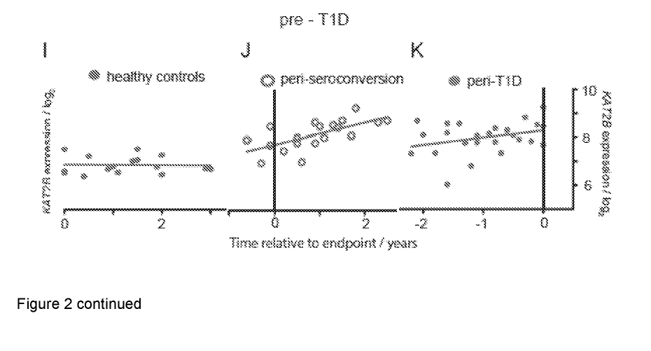

For the HCV data used in FIG. 2C a marked response was defined as an HCV titer decrease >3.5 log10iu/m1 and a poor response as an HCV titer decrease <1.5 log10iu/m1 by day 28 after commencing combined therapy with ribavirin and pegylated interferon-alpha. For the Malaria vaccine trial used in FIG. 2D ‘protection’ was defined as delayed or complete protection from subsequent confirmed P. Falciparum infection following standardised exposure (x5 bites) compared to infectivity control subjects. For the influenza data used in FIG. 2E protection was defined as >1=1 high response to at least 1 (of 3) included strains. A high response was defined as >1=4-fold increase in HAI titre at d28 and a titre >1=1:40 as per US FDA guidelines.

All gene expression data used has been deposited in publicly available repositories (NCBI-GEO and ArrayExpress): AAV, SLE (E-MTAB-2452, E-MTAB-157, E-MTAB-145) IBD (E-MTAB-331), LCMV (GSE9650), HCV (GSE7123), malaria vaccination (GSE18323), influenza vaccination (GSE29619), yellow fever vaccination (GSE13486), dengue fever (GSE25001), IPF (GSE28221), type 1 diabetes (E-TABM-666), NOD (GSE21897), RA (GSE15258, GSE33377), in vitro CD8 stimulation (E-MTAB-3470).

Data Analysis.

Preprocessing and quality control (QC).

For Mediante hs25k arrays, raw image data were extracted using Koadarray v2.4 software (Koada Technology) and probes with a confidence score >0.3 in at least one channel were flagged as present. Extracted data were imported into R where log transformation and background subtraction were performed followed by within array print-tip Loess normalization and between-array quantile and scale normalization using the Limma package³⁹ in Bioconductor⁴⁰. Further analysis was then performed in R and only data demonstrating a strong negative correlation (r²>0.9) between dye swap replicates were used in downstream analyses.

Affymetrix raw data (.CEL) files were imported into R and subjected to variance stabilization normalization using the VSN package in BioConductor⁴¹. Quality control was performed using the Bioconductor package arrayQualityMetrics⁴² with outlying samples removed from downstream analyses. Correction for batch variation was performed using the Bioconductor package ComBat⁴³ and batch structure was included as a covariate in downstream correlation analyses.

Clustering.

Hierarchical clustering was performed using a Pearson correlation distance metric and average linkage analysis, performed either in Cluster with visualization in Treeview⁴⁴, using Genepattern⁴⁵ or directly in R using hclust in the stats package.

Differential Expression

Differentially-expressed genes were identified using linear modeling and an empirical Bayes method³⁹ using a false discovery rate threshold of 0.05 as indicated to determine significance.

Weighted Gene Coexpression Network Analysis (WGCNA).

Highly correlated genes in immune cell subsets were identified and summarized with a modular eigengene profile using the Weighted Gene Coexpression Network Analysis (WGCNA) Bioconductor package in R⁴⁶. Normalized, log transformed expression data was variance filtered using the inflexion point of a ranked list of median absolute deviation values for all probes. A soft thresholding power was chosen based on the criterion of approximate scale-free topology⁴⁷. Gene networks were constructed and modules identified from the resulting topological overlap matrix with a dissimilarity correlation threshold of 0.01 used to merge module boundaries and a specified minimum module size of n=30. Modules were summarized as a network of modular eigengenes, which were then correlated with a matrix of clinical variables and the resulting correlation matrix visualized as a heatmap. As each module by definition is comprised of highly correlated genes, their combined expression may be usefully summarized by eigengene profiles⁴⁸, effectively the first principal component of a given module. A small number of eigengene profiles may therefore effectively ‘summarize’ the principle patterns within the cellular transcriptome with minimal loss of information. This dimensionality-reduction approach also facilitates correlation of ME with clinical traits. Significance of correlation between a given clinical trait and a modular eigengene was assessed using linear regression with Bonferroni adjustment to correct for multiple testing. Independent association of a given module eigengene or gene expression profile (e.g. KAT2B) with clinical outcome was assessed using a multiple linear regression model. Significance of each term in the linear model was plotted against its regression coefficient, as a measure of the strength of association (the regression coefficient reflecting the change in clinical outcome per unit change in modular/gene expression).

Overlap of signatures with modules derived from network analysis is shown to the right of selected module heatmaps by the following formula to allow correction for variable module size: (signature genes overlapping with module genes, n)/(genes in module, n) x100. The overlap of randomly selected signatures of equivalent size was used as a control and is shown adjacent to the above plots.

HOPACH Analysis

For validation purposes, highly-correlated genes were independently partitioned into discrete modules using a second algorithm, Hierarchical Ordered Partitioning And Collapsing Hybrid (HOPACH⁴⁹) in R. This approach differs from WGCNA in that it does not rely on a user-specified correlation threshold to define module boundaries but rather aims to maximize homogeneity of modules. Normalized, log transformed data were clustered using a hierarchical algorithm with modular boundaries defined by the median split silhouette (MSS), a measure of how well-matched a gene is to the other genes within its current cluster versus how well-matched it would be if it were moved to another cluster. On partitioning the dataset into clusters, each cluster is reiteratively subdivided until the MSS is maximized, thereby producing an optimal segregation into maximally discrete modules.

Knowledge-Based Network Generation and Pathway Analysis

The biological relevance of gene groups comprising modules identified by co-expression analysis were further investigated using the Ingenuity Pathways Analysis platform⁵⁰. Six modules from the CD4 T cell WGCNA analysis showed significant correlation with clinical outcome in AAV after correction for multiple testing (Bonferroni method). The inventors applied network and pathway enrichment analysis to genes comprising these modules to determine whether they may have any biological relevance. Briefly, for network analysis genes from a specified target set of interest are progressively linked together based on a measure of their interconnection, which is derived from described functional interactions. Additional highly interconnected genes that are absent from the target genes (open symbols) may be added to complete a network of arbitrary size (set at n=35). Networks may be ranked by significance which reflects the probability of randomly generating a network of similar size from genes included in the full knowledge database containing at least as many target genes as in the network in question. For pathways analysis, the overrepresentation of canonical pathways (pre-defined, well-characterized metabolic and signaling pathways curated from extensive literature reviews) amongst a specified set of target genes is assessed, with significance determined by computing a Fisher's exact test with false discovery rate correction for multiple testing.

Gene Set Enrichment Analysis (GSEA)

GSEA″ was used to further assess whether specific biological pathways or signatures were significantly enriched between patient subgroups identified by gene modules (as opposed to testing for enrichment of pathways within modules themselves as outlined in the previous section). GSEA determines whether an a priori defined ‘set’ of genes (such as a signature) show statistically significant cumulative changes in gene expression between phenotypic subgroups (such as patients with relapsing or quiescent disease). In brief, all genes are ranked based on their differential expression between two groups then an enrichment score (ES) is calculated for a given gene set based on how often its members appear at the top or bottom of the ranked differential list. 1000 random permutations of the phenotypic subgroups were used to establish a null distribution of ES against which a normalized enrichment score (NES) and FDR-corrected q values were calculated. GSEA was run with a focused subgroup of gene signaturesⁱⁱ selected to test the null hypothesis that different CD8 T cell phenotypes were not significantly enriched in patient subgroups identified by modular analysis.

Selection of Optimal PBMC-Level Biomarkers.

Optimal surrogate markers facilitating identification of the CD4 T cell co-stimulation/CD8 exhaustion signatures in PBMC-level data were determined using a randomforests classification algorithm⁵¹ (FIG. 2A). Although signatures apparent in purified T cell transcriptome data correlate with clinical outcome, they cannot be similarly detected in data derived from PBMC due to the confounding influence of expression from other cell types nor can the same genes be used to predict outcome in PBMC²′²°. However, as CD4 T cell co-stimulation and CD8 T cell exhaustion signatures themselves showed close correlation the inventors hypothesized that this would amplify the signal detectable in PBMC and that detection of the combined CD4/CD8 T cell response may be feasible. The availability of both separated T cell and PBMC data from the same patients at the same time facilitate a supervized search for surrogate markers of the co-stimulation/exhaustion signatures in PBMC. Expression data derived from both CD4 T cells and PBMC were available for a cohort of n=37 patients (AAV and SLE) following QC and hybridization to the HsMediante25k custom microarray platform and constituted a training cohort. Normalized, log-transformed expression data was analyzed using the MLlnterfaces Bioconductor package in R⁵². Using PBMC-level expression data samples were classified into subgroups showing either high or low expression of the costimluation/exhaustion signature and probes were subsequently ranked using the variable importance metric based on their ability to predict allocation to either group. The variable importance for a given gene reflects the change in accuracy of classification (% increase in MSE or increase in node purity) when that variable is randomly permuted. For a poorly predictive gene, random permutation of its values will minimally influence classification accuracy. Conversely, the most robust predictors will have a comparatively large effect on classification accuracy when randomly permuted. PBMC samples from a subset of n=37 cases derived from the training cohort were labeled and hybridized on an alternative microarray platform (Affymetrix Gene ST1.0) as a technical validation set (FIG. 2B, left panel). PBMC samples from an independent n=47 cases not included in the training cohort were labeled and hybridized to the Affymetrix Gene ST1.0 platform as an independent test set (FIG. 2B, right panel). For both technical validation and independent test sets expression of the optimal biomarker identified in FIG. 2A (KAT2B) was used to bisect the cohort relative to the median expression and clinical outcome was compared in KAT2Bhi and KAT2Blo patients.

Linear Models

Linear modeling was performed in R using the stats package. This took the form of fit <−Im(y−x1+×2+×3, data=mydata)

where y (the response variable) was selected as normalized flare rate (flares/days follow-up) and x₁-x, (the test variables) were selected to include measures of disease activity (both clinical scores and laboratory markers of inflammation), quantification of circulating leucocyte subsets (lymphocytes, neutrophils) and concurrent measurements of autoantibody titer where relevant. Test variables also included a biomarker profile (e.g. exhaustion signature or KAT2B expression). The significance and magnitude (regression coefficient, reflecting change in response variable (flares/days follow-up) per unit change in each test variable included) were extracted and plotted against each other. Not all clinical or laboratory measures were relevant comparisons in each case and therefore were not all included in every model generated.

T Cell Culture

Primary human CD8 T cells were separated from leucocyte cones obtained from NHS Blood and Transplant (Addenbrooke's Hospital, Cambridge, UK) by centrifugation over ficoll and positive selection using magnetic beads as previously described²⁰. The purity of separated cell subsets was determined by three-color flow cytometry. Purified T cells were labeled with 10 pM CFSE (Invitrogen) and resuspended in complete RPMI 1640 (Sigma Aldrich) in the presence of 10% FCS. Purified CD8⁺ T cells (>95%) were then stimulated in sterile, 96-well U-bottomed culture plates (Greiner) using an ‘artificial APC’ consisting of MACS iBead particles (1:2 bead:cell ratio, Miltenyi) or DyneBead particles (Invitrogen) conjugated to either CD3/CD28 or CD2/CD3/CD28 as indicated in the presence of IL2 (10 ng/ml, Gibco life technologies) for 6 days. The magnetic iBead construct was removed after 36h in some instances as indicated. In some experiments, additional costimulation was provided by the addition of either IFNa (lOng/ml, Abcam) or by additional conjugation of recombinant Human PD-L1 Fc Chimera (life technologies, 1 pg/ml) or anti-CD40 antibody (50 ng/ml, Abcam) as indicated. The nature of costimulatory signals tested was based upon the results of the network analysis of CD4 T cell modules described above.

For restimulation experiments cells were harvested on day 6 post-stimulation and sorted into IL7R^{hi} and IL7R^{lo} populations using a FACSArialll cell sorter (BD Biosciences) with live/dead discrimination performed using an AquaFluorescent amine-reactive dye (Invitrogen). Cell numbers were normalized and were resuspended in complete RPMI 1640 (2×104/ml, Sigma-Aldrich) and ‘rested’ in a sterile, U-bottomed culture plate (Greiner) for 6 days (37° C., 5% CO2) before being restimulated (anti-CD2/3/28 1:2 bead:cell ratio, Miltenyi MACSiBead) for a further 6 days in the presence of IL2 (10 ng/ml, Gibco life technologies). Note that, human memory CD8 T cell subsets do not equivalently respond to the stimulation conditions described above. As primary whole human CD8 T cells are composed of highly variable proportions of memory subsets and whole CD8 T cells were stimulated it was necessary to perform paired tests of significance when comparing resulting T cell subsets and transcriptional profiles.

Flow Cytometry.

Immunophenotyping was performed using an LSR Fortessa analyzer (BD Biosciences), and data was analyzed using FlowJo software (Tree Star). Reactions were standardized with multicolor calibration particles (BD Biosciences) with saturating concentrations of the following antibodies: AquaFluorescent Live/Dead (Invitrogen), IL7Ra AF647 (BD biosciences, clone HIL-7R-M21), PDCD1 APC (eBioscience, clone MIH4). For intracellular staining, cells were fixed and permeabilized using a transcription factor staining buffer set (eBioscience) and before staining with saturating concentrations of antibody against BCL2 (BD Biosciences, clone 100).

Results

The clinical course of autoimmune and infectious disease varies greatly even between individuals with the same condition. An understanding of the molecular basis for this heterogeneity could lead to significant improvements in both monitoring and treatment ENREF 1. During chronic infection the process of T cell exhaustion inhibits the immune response, facilitating viral persistence'. The inventors show that a transcriptional signature reflecting CD8 T cell exhaustion is associated with poor clearance of chronic viral infection, but conversely predicts better prognosis in multiple autoimmune diseases. The development of CD8 T cell exhaustion during chronic infection is driven by both persistence of antigen and a lack of accessory ‘help’ signals. In autoimmunity, the inventors found that where evidence of CD4 T cell costimulation was pronounced, that of CD8 T cell exhaustion was reduced.

The inventors could further reproduce the exhaustion signature by modifying the balance of persistent TCR stimulation and specific CD2-induced costimulation provided to human CD8 T cells in vitro, suggesting that each process plays a role in dictating outcome in autoimmune disease. The “non-exhausted” T cell state driven by CD2-induced costimulation was reduced by signals through the exhaustion-associated inhibitory receptor PD-1, suggesting that induction of exhaustion may be a therapeutic strategy in autoimmune disease and infection-associated immunopathology. Using expression of optimal surrogate markers of costimulation/exhaustion signatures in independent datasets, the inventors confirmed an association with good clinical outcome or response to therapy in infection (hepatitis C virus (HCV)), and vaccination (yellow fever, malaria, influenza) but poor outcome in autoimmune and infection-associated immunopathology (type 1 diabetes (T1D), anti-neutrophil cytoplasmic antibody (ANCA)-associated vasculitis (AAV), systemic lupus erythematosus (SLE), idiopathic pulmonary fibrosis (IPF) and dengue hemorrhagic fever (DHF)). Thus, T cell exhaustion plays a central role in determining outcome in autoimmune disease and targeted manipulation of this process could lead to new therapeutic opportunities.

In a complex set of data such as the transcriptome, similar measurements may be grouped together by network analysis to form discrete modules ENREF 2 ENREF 3 ENREF 4that can highlight novel pathways contributing to the pathogenesis of complex diseases ENREF 5. The inventors have previously shown that a CD8 T cell transcriptional signature in patients with multiple immune-mediated diseases can predict a subsequent relapsing disease²′³. However, the biology underlying this observation was not clear. The inventors therefore applied weighted gene co-expression network analysis³ to the transcriptomes of purified CD4 and CD8 T cells isolated from a prospective cohort of 44 AAV patients with active, untreated disease⁷ to further explore the mechanisms driving relapsing autoimmunity. Modules of genes were summarized as ‘eigengene’ profiles that were correlated with clinical variables and visualized in the form of a heatmap. Modules derived from both CD8 and CD4 T cell transcriptomes showed strong correlation with disease outcome but not activity, and were co-correlated despite being mutually exclusive. A similar analysis using a cohort of 23 SLE patients also presenting with active, untreated disease² identified analogous CD8 and CD4 T cell expression modules that again correlated with clinical outcome but not disease activity. By contrast a type 1 interferon response signature was associated with disease activity but not with long-term outcome, consistent with previous reports⁴.

Next, the inventors reasoned that genes within co-correlated modules in related cell types might inform the biology of relapsing disease. By selecting CD4 T cell modules showing significant, strong correlation with relapse rate and performing network enrichment analysis the inventors identified a module corresponding to CD4 T cell costimulation. By way of validation the inventors repeated this analysis using an independent co-expression network algorithm that similarly demonstrated association between a CD4 costimulation module and clinical outcome. The independent association of modular signatures with clinical outcome was confirmed using multiple linear regression modeling and was only apparent during active disease.

During chronic viral infection CD8 T cell memory responses are exquisitely dependent on CD4 T cell costimulation” which can lead to the resolution of chronic infection in both mice′ and humans'. When antigen persists in the absence of costimulation CD8 T cells become ‘exhausted’, a phenotype characterized by progressive loss of effector function, persistent high expression of inhibitory receptors and profound changes in gene expression, distinct from those seen in effector, memory or anergic T cells⁸. Although mice lacking inhibitory receptors have an increased incidence and severity of autoimmunity⁸′^{1o} a specific role for exhaustion in dictating the outcome of autoimmune responses has not been demonstrated.

The inventors hypothesized that CD4 T cell signals may be important in limiting exhaustion towards persistent self-antigen during autoreactive immunity, analogous to responses during persistent infection. The inventors therefore used Gene Set Enrichment Analysis (GSEA¹¹) to test for altered expression of transcriptional signatures reflecting T cell exhaustion (and other T cell-related phenotypes) between patient subgroups defined by the CD8 modular analysis, who go on to develop relapsing or quiescent autoimmunity. Using this approach, the inventors observed that genes specifically downregulated in exhausted CD8 T cells during chronic murine LCMV infection (but not altered in memory, naïve or effector cells⁸) were similarly downregulated in CD8 T cells from patients at low risk of subsequent relapse.

During chronic murine LCMV infection, T cell exhaustion is driven by coordinate upregulation of multiple coinhibitory receptors¹² that signal synergistically to produce a state of generalized immunosuppression¹³. In autoimmunity, these receptors were not coordinately upregulated as a group. Instead patients with good prognosis from each disease were characterized by upregulation of a distinct subset of exhaustion-associated coinhibitory receptors. Although a divergence from the murine LCMV model, T cell exhaustion accompanied by expression of a limited subset of coinhibitory receptors is similar to that described in intratumoral CD8 T cells¹⁴ which are a target for checkpoint there^{py15,16}.

To confirm whether exhaustion was associated with clinical outcome, the inventors used the murine CD8 T cell exhaustion signature⁸ to perform unsupervized hierarchical clustering of three independent cohorts of patients with distinct diseases (AAV, SLE, IBD). In each case this identified a subgroup of patients with both early and recurrent relapses. Whereas CD8 exhaustion was associated with poor outcome in viral infection, in every case it predicted favorable prognosis in autoimmune and infection-associated immunopathology. Again, independent association with outcome was confirmed using multiple linear regression models. Together, these data demonstrate that a transcriptional signature of relative CD8 T cell exhaustion, similar to that determining outcome in chronic viral infection and cancer, is apparent during active, untreated disease in patients with favorable long-term outcome in multiple autoimmune and inflammatory diagnoses.

CD8 T cell exhaustion is characterized by high expression of coinhibitory receptors (such as PD-1¹²) and low expression of nascent memory markers (such as IL7R¹⁷) and is promoted by both the persistence of antigen¹⁸ and a lack of accessory costimulation⁶. To understand signals driving exhaustion and outcome in autoimmunity, the inventors attempted to recreate the outcome-associated transcriptional signatures using variable TCR signal duration and costimulation of primary human cells in vitro. The inventors stimulated purified human CD8 T cells using a magnetic bead conjugated with antibodies targeting costimulatory molecules (FIG. 1A) and measured expression of IL7R and PD-1 expression (FIG. 1B-D) as markers indicating an exhausted phenotype. Comparison between persistent (6 days) and transient TCR stimulation (36 hours) showed that IL7R expression returned on a proportion of cells after several divisions when the TCR stimulus was removed but failed to do so if it persisted (FIG. 1B). The inventors then systematically tested whether costimulatory molecules, identified from the CD4 T cell network analysis described above FIGS. 1 C-D), could overcome the effect of persistent TCR stimulation during in vitro differentiation. The inventors found that specific costimulation with anti-CD2 (FIG. 1B), but not with other stimuli such as IFNa or anti-CD40, resulted in maintained IL7R expression, limited upregulation of PD-1 and enhanced cell survival.

While CD8 exhaustion is known to limit viral control during chronic infection, exhausted cells may be restored to useful function by blocking inhibitory signaling through PD-1¹⁶. Enhancing coinhibitory signals is therefore a logical therapeutic strategy in autoimmune disease, aiming to facilitate exhaustion despite high levels of costimulation that would otherwise be predicted to result in an aggressive relapsing disease course. To test this concept, primary human CD8 T cells were costimulated with anti-CD2 during persistent TCR signaling as above (FIG. 1C) in the presence or absence of a bead-bound Fc-chimeric version of the principal PD-1 ligand, PDL-1 (FIG. 1D). When added to CD2-costimulated CD8 T cell cultures, increased

PD-1/PDL-1 signaling suppressed differentiation of a non-exhausted IL7R^{hi} subpopulation (FIG. 1C-D).

To define the phenotype of T cell exhaustion more robustly, as small numbers of surface markers are insufficient, the inventors analyzed the transcriptome of CD8 T cells exposed to persistent stimulation with and without CD2 signaling. This CD2 response signature characterized exhausted cells but not effector or memory subsets (by GSEA). Consistent with this, patient clusters generated using the CD2 response signature recreated subgroups similar to those generated using the murine LCMV CD8 exhaustion signature. Thus, CD2 signaling during persistent TCR stimulation of primary human CD8 T cells prevents the development of transcriptional changes characteristic of exhaustion, recreating transcriptional signatures associated with outcome in both viral infection and autoimmunity.

To confirm that the transcriptional signatures reflected the development of functional exhaustion in vitro, the inventors showed that cells appearing exhausted by surface markers (IL7R^{1o}PD-1^{hi}) also expressed markers of apoptotic resistance, characteristic cytokine patterns and showed diminished survival on restimulation (BCL2^{1o}IFNy^{io}1L10^{hi}). There was no evidence of preferential accumulation of CD8 T cell subsets following CD2-induced costimulation. These data highlight the importance of CD2 signaling in limiting the development of CD8 T cell exhaustion in the face of persistent TCR simulation, and provide a starting point for more sophisticated attempts to therapeutically exhaust an autoimmune response in a targeted fashion.

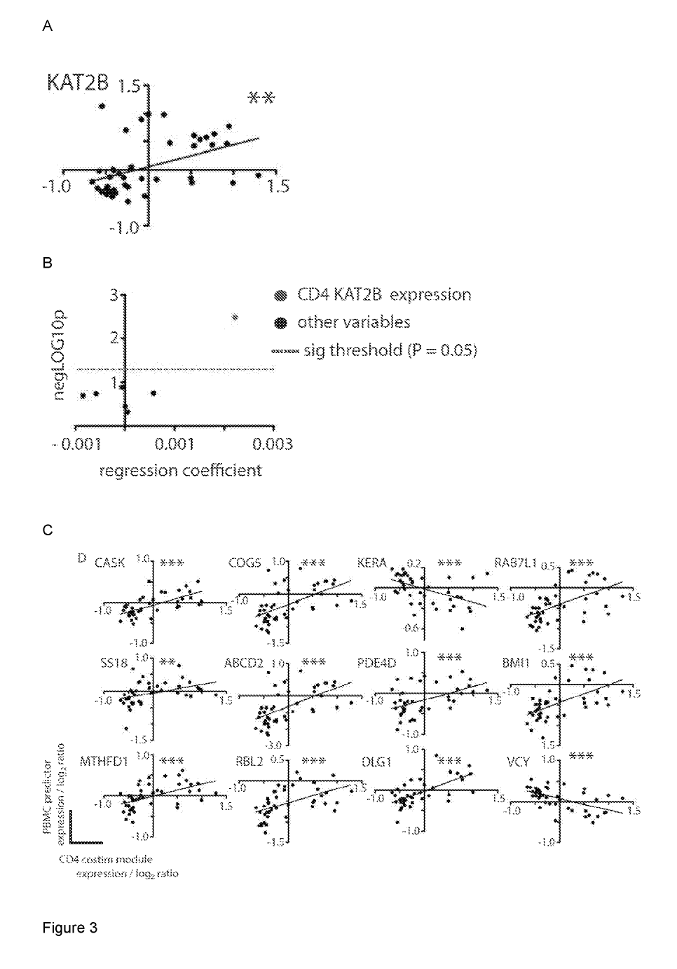

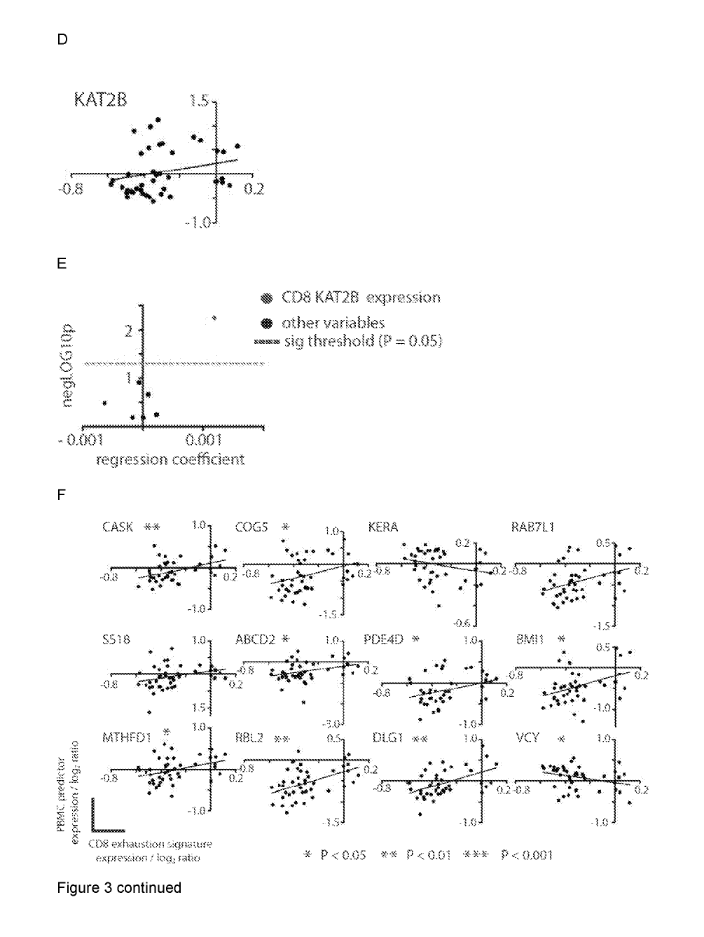

The inventors next aimed to independently validate the association between the balance of CD4 costimulation and CD8 exhaustion with clinical outcome using published datasets. The majority of these profile unseparated peripheral blood mononuclear cells (PBMC), in which T cell-intrinsic signatures are not readily apparent due to the confounding influence of expression from other cell types²⁰. The inventors therefore used a classification algorithm (randomforests) to identify optimal surrogate markers of costimulation/exhaustion modules in PBMC data from autoimmune patients taken concurrently with the T cells described above (FIG. 2A). As the CD8 exhaustion and CD4 costimulation signatures were themselves correlated, it became easier to detect their combined signal in PBMC using surrogate markers (FIG. 2A, FIG. 3). The top-ranked candidate KAT2B is a transcriptional co-activator known to mediate an anti-apoptotic effect under conditions of metabolic stress⁵² and to increase cellular resistance to cytotoxic compounds⁵³. These characteristics, along with its high expression in memory and T-follicular helper and NK cells, suggest that it may mark the development of a durable, persistent T cell phenotype promoting long-lived responses in either infection or autoimmunity. The observed association was confirmed by both technical replication (using the same samples run on an independent array platform) and independent validation (FIG. 2B).

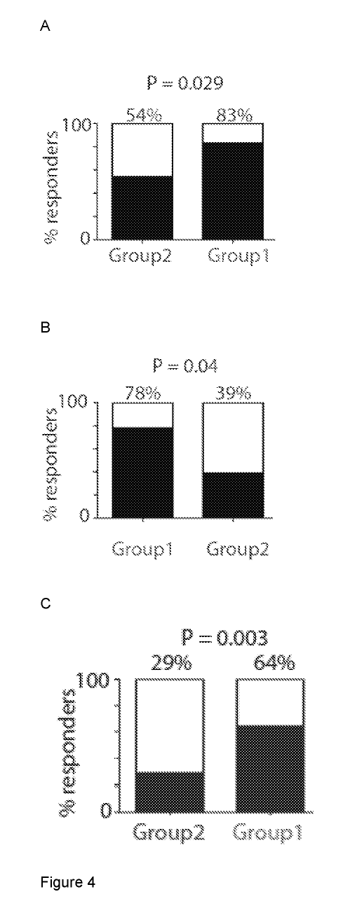

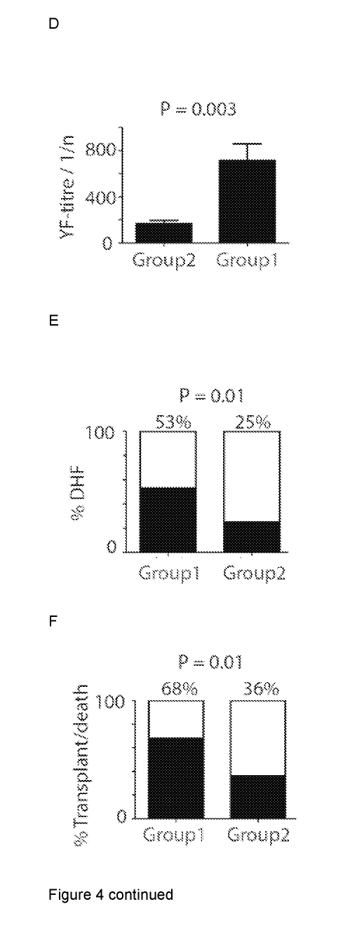

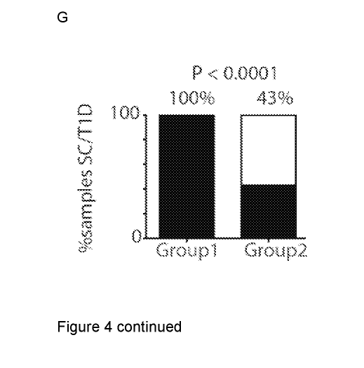

ENREF 25 To test whether similar associations may be apparent in multiple infectious and autoimmune diseases the inventors directly compared expression levels of KAT2B (and of the other top surrogate markers, FIG. 4) between clinical subgroups defined within published studies for which PBMC expression and linked clinical outcome data were available. Where subgroups were not pre-specified, the inventors compared clinical outcome in groups stratified as having either above or below-median expression of KAT2B (FIG. 2C-K). Hierarchical clustering using all top surrogate markers gave similar stratification to that seen using KAT2B alone, while as expected the separation of patient subgroups varied slightly in different clinical circumstances (FIG. 2 C-K, FIG. 4).

ENREF 26 ENREF 32 Combined interferon and ribavirin therapy may result in increased virus-specific T cell responses in chronic HCV, although such eradication therapy is successful in only 50% of cases²¹ and in some cases no change in endogenous immune response is observed²². In a cohort of hepatitis C patients receiving combination therapy, KAT2B expression was progressively induced and showed significantly greater induction in patients ultimately responding to therapy (FIG. 2C). In a clinical trial of malaria vaccination²³ high KAT2B expression identified a subgroup with response rates of 78%, almost twice that seen in the low response group (FIG. 2D). Moreover, response to vaccination for either influenza²⁴ (FIG. 2E) or yellow fever²⁵ (FIG. 2F) could be predicted by stratifying recipients based on their expression of KAT2B following vaccine exposure. Dengue viral infection can result in a wide range of clinical manifestations ranging from asymptomatic infection or self-limiting fever (uncomplicated dengue, UD) to hemorrhagic fever (DHF). Consistent with our observations in autoimmunity, the inventors observed that KAT2B expression was elevated in patients developing the excessive inflammatory response of DHF (FIG. 2G)²⁶.

The inventors next asked whether surrogate detection of T cell costimulation/exhaustion modules could predict progression of other autoimmune diseases. Idiopathic pulmonary fibrosis (IPF) is a progressive interstitial lung disease characterized by both autoantibodies and autoreactive CD4 T cells²⁷. In a cohort of 75 IPF patients²⁸ high expression of KAT2B predicted subsequent progression to transplantation or death (FIG. 2H).The inventors also observed that PBMC Kat2b expression was elevated in the murine NOD model of type 1 diabetes²⁹ with levels rising sharply during the T cell initiation phase, long before the onset of diabetic hyperglycemia ENREF 43. In a cohort of samples taken prospectively from children at high risk of disease but prior to its onset³⁹ expression of KAT2B was seen to specifically and progressively rise (FIG. 21-K) both in patients who progressed to type 1 diabetes and in those who developed islet-cell autoantibodies.

ENREF 47 ENREF 10 ENREF 48 ENREF 49 ENREF 49 The inventors show that the balance between costimulatory and coinhibitory signals that shape T cell exhaustion coincide with opposite clinical outcomes during autoreactive and anti-viral immunity. This at once allows prediction of outcome during infection and autoimmunity and creates the potential for targeted therapeutic exhaustion of an autoimmune response in those predicted to follow an aggressive disease course. That this association is apparent in multiple autoimmune and infection-associated immunopathologies emphasizes the importance of signals shaping T cell exhaustion in driving risk of relapse or recurrence (prognosis) rather than disease susceptibility (diagnosis) or immediate severity (disease activity), and suggests that targeted manipulation of these processes may lead to new treatment strategies that extend beyond the conditions discussed here.

TABLE 1

| | | | | | | Upregulated/downregulated in |
|---|---|---|---|---|---|---|
| | | GenBank | | SEQ | | exhausted CD8⁺ T cell/lack of |
| | Gene | accession | GenBank | ID | | CD4⁺ T cell costimulation |
| No | symbol | no. | version no. | NO. | Description | phenotype |
| 1 | KAT2B | NM_003884 | GI: 156071487 | 1 | K(lysine) acetyltransferase 2B gene | downregulated |
| 2 | CASK | NM_003688 | GI: 193788694 | 2 | calcium/calmodulin-dependent serine protein kinase 3 gene | downregulated |
| 3 | ABCD2 | NM_005164 | GI: 168480147 | 3 | ATP-binding cassette sub-family D member 2 gene | downregulated |
| 4 | DLG1 | NM_004087 | GI: 148539577 | 4 | disks large homolog 1 gene | downregulated |
| 5 | SS18 | NM_005637 | GI: 815891164 | 5 | synovial sarcoma translocation, chromosome 18 gene | downregulated |
| 6 | RBL2 | NM_005611 | GI: 172072596 | 6 | Retinoblastoma-like protein 2 gene | downregulated |
| 7 | RAB7L1 | NM_003929 | GI: 208609960 | 7 | RAS oncogene family-like 1 gene | downregulated |
| 8 | MTHFD1 | NM_005956 | GI: 222136638 | 8 | methylenetetrahydrofolate dehydrogenase 1 gene | downregulated |
| 9 | KERA | NM_007035 | GI: 62865891 | 9 | keratocan gene | upregulated |
| 10 | BMI1 | NM_005180 | GI: 323462179 | 10 | B cell-specific Moloney murine leukemia virus integration site 1 | downregulated |
| | | | | | gene | |
| 11 | COG5 | NM_006348 | GI: 240849530 | 11 | conserved oligomeric Golgi complex subunit 5 gene | downregulated |
| 12 | PDE4D | NM_006203 | GI: 157277986 | 12 | cAMP-specific 3′,5′-cyclic phosphodiesterase 4D gene | downregulated |
| 13 | VCY | NM_004679 | GI: 49355825 | 13 | variable charge, Y-linked gene | upregulated |

### REFERENCES

All documents mentioned in this specification are incorporated herein by reference in their entirety.

1. Wherry, E. J. T cell exhaustion. Nature immunology 12, 492-499 (2011).

2. McKinney, E. F., et al. A CD8⁺ T cell transcription signature predicts prognosis in autoimmune disease. Nature medicine 16, 586-591 (2010).

3. Lee, J. C., et al. Gene expression profiling of CD8⁺ T cells predicts prognosis in patients with Crohn disease and ulcerative colitis. The Journal of clinical investigation 121, 4170-4179 (2011).

4. Baechler, E. C., et al. Interferon-inducible gene expression signature in peripheral blood cells of patients with severe lupus. Proceedings of the National Academy of Sciences of the United States of America 100, 2610-2615 (2003).

5. West, E. E., et al. Tight regulation of memory CD8(+) T cells limits their effectiveness during sustained high viral load. Immunity 35, 285-298 (2011).

6. Aubert, R. D., et al. Antigen-specific CD4 T-cell help rescues exhausted CD8 T cells during chronic viral infection. Proceedings of the National Academy of Sciences of the United States of America 108, 21182-21187 (2011).

7. Quigley, M., et al. Transcriptional analysis of HIV-specific CD8⁺ T cells shows that PD-1 inhibits T cell function by upregulating BATF. Nature medicine 16, 1147-1151 (2010).

8. Wherry, E. J., et al. Molecular signature of CD8⁺ T cell exhaustion during chronic viral infection. Immunity 27, 670-684 (2007).

9. Rangachari, M., et al. Bat3 promotes T cell responses and autoimmunity by repressing Tim-3-mediated cell death and exhaustion. Nature medicine 18, 1394-1400 (2012).

10. Francisco, L. M., Sage, P. T. & Sharpe, A. H. The PD-1 pathway in tolerance and autoimmunity. Immunological reviews 236, 219-242 (2010).

11. Subramanian, A., et al. Gene set enrichment analysis: a knowledge-based approach for interpreting genome-wide expression profiles. Proceedings of the National Academy of Sciences of the United States of America 102, 15545-15550 (2005).

12. Blackburn, S. D., et al. Coregulation of CD8⁺ T cell exhaustion by multiple inhibitory receptors during chronic viral infection. Nature immunology 10, 29-37 (2009).

13. Sevilla, N., et al. Immunosuppression and resultant viral persistence by specific viral targeting of dendritic cells. The Journal of experimental medicine 192, 1249-1260 (2000).

14. Virgin, H. W., Wherry, E. J. & Ahmed, R. Redefining chronic viral infection. Cell 138, 30-50 (2009).

15. Gubin, M. M., et al. Checkpoint blockade cancer immunotherapy targets tumour-specific mutant antigens. Nature 515, 577-581 (2014).

16. Baitsch, L., et al. Exhaustion of tumor-specific CD8(+) T cells in metastases from melanoma patients. The Journal of clinical investigation 121, 2350-2360 (2011).

17. Lang, K. S., et al. Inverse correlation between IL-7 receptor expression and CD8 T cell exhaustion during persistent antigen stimulation. European journal of immunology 35, 738-745 (2005).

18. Mueller, S. N. & Ahmed, R. High antigen levels are the cause of T cell exhaustion during chronic viral infection. Proceedings of the National Academy of Sciences of the United States of America 106, 8623-8628 (2009).

19. Barber, D. L., et al. Restoring function in exhausted CD8 T cells during chronic viral infection. Nature 439, 682-687 (2006).

20. Lyons, P. A., et al. Microarray analysis of human leucocyte subsets: the advantages of positive selection and rapid purification. BMC Genomics 8, 64 (2007).

21. Taylor, M. W., et al. Changes in gene expression during pegylated interferon and ribavirin therapy of chronic hepatitis C virus distinguish responders from nonresponders to antiviral therapy. Journal of virology 81, 3391-3401 (2007).

22. Lauer, G. M., et al. Full-breadth analysis of CD8⁺ T-cell responses in acute hepatitis C virus infection and early therapy. Journal of virology 79, 12979-12988 (2005).

23. Vahey, M. T., et al. Expression of genes associated with immunoproteasome processing of major histocompatibility complex peptides is indicative of protection with adjuvanted RTS,S malaria vaccine. The Journal of infectious diseases 201, 580-589.

24. Nakaya, H. I., et al. Systems biology of vaccination for seasonal influenza in humans. Nature immunology 12, 786-795 (2010).

25. Querec, T. D., et al. Systems biology approach predicts immunogenicity of the yellow fever vaccine in humans. Nature immunology 10, 116-125 (2009).

26. Hoang, L. T., et al. The early whole-blood transcriptional signature of dengue virus and features associated with progression to dengue shock syndrome in Vietnamese children and young adults. Journal of virology 84, 12982-12994 (2010).

27. Shum, A. K., et al. BPIFB1 Is a Lung-Specific Autoantigen Associated with Interstitial Lung Disease. Science translational medicine 5, 206ra139 (2013).

28. Herazo-Maya, J. D., et al. Peripheral blood mononuclear cell gene expression profiles predict poor outcome in idiopathic pulmonary fibrosis. Science translational medicine 5, 205ra136 (2013).

29. Kodama, K., et al. Tissue- and age-specific changes in gene expression during disease induction and progression in NOD mice. Clinical immunology 129, 195-201 (2008).

30. Elo, L. L., et al. Early suppression of immune response pathways characterizes children with prediabetes in genome-wide gene expression profiling. Journal of autoimmunity 35, 70-76 (2010).

31. Stone, J. H., et al. A disease-specific activity index for Wegener's granulomatosis: modification of the Birmingham Vasculitis Activity Score. International Network for the Study of the Systemic Vasculitides (INSSYS). Arthritis and rheumatism 44, 912-920 (2001).

32. Tan, E. M., et al. The 1982 revised criteria for the classification of systemic lupus erythematosus. Arthritis and rheumatism 25, 1271-1277 (1982).

33. Isenberg, D. A., et al. BILAG 2004. Development and initial validation of an updated version of the British Isles Lupus Assessment Group's disease activity index for patients with systemic lupus erythematosus. Rheumatology 44, 902-906 (2005).

34. Silverberg, M. S., et al. Toward an integrated clinical, molecular and serological classification of inflammatory bowel disease: report of a Working Party of the 2005 Montreal World Congress of Gastroenterology. Can J Gastroenterol 19 Suppl A, 5A-36A (2005).

35. Harvey, R. F. & Bradshaw, M. J. Measuring Crohn's disease activity. Lancet 1, 1134-1135 (1980).

36. Walmsley, R. S., Ayres, R. C., Pounder, R. E. & Allan, R. N. A simple clinical colitis activity index. Gut 43, 29-32 (1998).

37. Whitney, A. R., et al. Individuality and variation in gene expression patterns in human blood. Proceedings of the National Academy of Sciences of the United States of America 100, 1896-1901 (2003).

38. Le Brigand, K., et al. An open-access long oligonucleotide microarray resource for analysis of the human and mouse transcriptomes. Nucleic Acids Res 34, e87 (2006).

39. Smyth, G. K. Linear models and empirical bayes methods for assessing differential expression in microarray experiments. Stat Appl Genet Mol Biol 3, Article3 (2004).

40. Gentleman, R. C., et al. Bioconductor: open software development for computational biology and bioinformatics. Genome Biol 5, R80 (2004).

41. Huber, W., von Heydebreck, A., Sultmann, H., Poustka, A. & Vingron, M. Variance stabilization applied to microarray data calibration and to the quantification of differential expression. Bioinformatics 18 Suppl 1, S96-104 (2002).

42. Kauffmann, A., Gentleman, R. & Huber, W. arrayQualityMetrics—a bioconductor package for quality assessment of microarray data. Bioinformatics 25, 415-416 (2009).

43. Johnson, W. E., Li, C. & Rabinovic, A. Adjusting batch effects in microarray expression data using empirical Bayes methods. Biostatistics 8, 118-127 (2007).

44. Eisen, M. B., Spellman, P. T., Brown, P. O. & Botstein, D. Cluster analysis and display of genome-wide expression patterns. Proceedings of the National Academy of Sciences of the United States of America 95, 14863-14868 (1998).

45. Renshaw, B. R., et al. Humoral immune responses in CD40 ligand-deficient mice. The Journal of experimental medicine 180, 1889-1900 (1994).

46. Langfelder, P. & Horvath, S. WGCNA: an R package for weighted correlation network analysis. BMC Bioinformatics 9, 559 (2008).

47. Barabasi, A. L. & Albert, R. Emergence of scaling in random networks. Science 286, 509-512 (1999).

48. Langfelder, P. & Horvath, S. Eigengene networks for studying the relationships between co-expression modules. BMC systems biology 1, 54 (2007).

49. Pollard, K.S.a.v.d.L.M.J. A new algorithm for hybrid hierarchical clustering with visualization and the bootstrap. http://cran.fhcrc.org/web/packaqes/hopach/index.htm.

50. Ingenuity Systems htlp://www.ingenuity.com.

51. Breiman, L. Random Forests. Machine Learning Journal 45, 5-32 (2001).

52. Vince Carey, R. G., Jess Mar, and contributions from Jason Vertrees and Laurent Gatto. MLInterfaces: Uniform interfaces to R machine learning procedures for data in Bioconductor containers. R package version 1.40.0.

53. Cramp, M. E., et al. Hepatitis C virus-specific T-cell reactivity during interferon and ribavirin treatment in chronic hepatitis C. Gastroenterology 118, 346-355 (2000).

1. A method of assessing whether an individual has an exhausted CD8⁺ T cell or lack of CD4⁺ T cell costimulation phenotype, the method comprising establishing, by determining the expression level of two more genes selected from the group consisting of:
K(lysine) acetyltransferase 2B gene (KAT2B);
calcium/calmodulin-dependent serine protein kinase 3 gene (CASK);
ATP-binding cassette sub-family D member 2 gene (ABCD2);
disks large homolog 1 gene (DLG1);
synovial sarcoma translocation, chromosome 18 gene (SS18);
Retinoblastoma-like protein 2 gene (RBL2);
RAS oncogene family-like 1 gene (RAB7L1);
methylenetetrahydrofolate dehydrogenase 1 gene (MTHFD1);
keratocan gene (KERA);
B cell-specific Moloney murine leukemia virus integration site 1 gene (BMI1);
conserved oligomeric Golgi complex subunit 5 gene (COG5);
cAMP-specific 3′,5′-cyclic phosphodiesterase 4D gene (PDE4D); and
variable charge, Y-linked gene (VCY);
in a sample obtained from the individual, whether said subject has said phenotype,
wherein said phenotype is characterised by downregulated expression of genes KAT2B, CASK, ABCD2, DLG1, SS18, R MTHFD1, BMI1, COG5, and PDE4D, and upregulated expression of genes KERA and VCY, relative to the level of expression of these genes in an individual who does not have said phenotype.

2. A method of assessing whether an individual is at high risk or low risk of autoimmune disease progression, the method comprising either:
(A) determining whether the individual has an exhausted CD8⁺ T cell or lack of CD4⁺ T cell costimulation phenotype using the method of claim 1,
wherein the presence of said phenotype indicates that the individual is at low risk of autoimmune disease progression, and
wherein the absence of said phenotype indicates that the individual is at high risk of autoimmune disease progression; OR:
(B) determining whether the individual has or does not have an exhausted CD8⁺ T cell or lack of CD4⁺ T cell costimulation phenotype, the method comprising:
(i) providing a PBMC sample obtained from the individual;
(ii) extracting mRNA from the PBMC sample;
(iii) performing reverse transcription quantitative PCR (RT-qPCR) to convert the mRNA into cDNA and determine the expression level of two more genes selected from the group consisting of: KAT2B, CASK, ABCD2, DLG1, SS18, RBL2, RAB7L1, MTHFDI, KERA, BMI1, COG5, PDE4D, and VCY,
wherein said phenotype is characterised by downregulated expression of genes KAT2B, CASK, ABCD2, DLG1, SS18, RBL2, RAB7L1, MTHFDI, BMI1, COG5, and PDE4D, and upregulated expression of genes KERA and VCY, relative to the level of expression of these genes in an individual who does not have said phenotype, and
wherein the presence of said phenotype indicates that the individual is at low risk of autoimmune disease progression, and
wherein the absence of said phenotype indicates that the individual is at high risk of autoimmune disease progression.

3. The method according to claim 2, wherein the autoimmune disease is not rheumatoid arthritis (RA) or inflammatory bowel disease (IBD).

4. The method according to claim 3, wherein the autoimmune disease is selected from the group consisting of: ANCA-associated vasculitis (AAV), systemic lupus erythematosus (SLE), type 1 diabetes, and idiopathic pulmonary fibrosis (IPF).

5. The method according to claim 4, wherein the autoimmune disease is AAV or SLE, and wherein an individual who is at low risk of autoimmune disease progression is at low risk of relapses or flares of the disease, and wherein an individual who is at high risk of autoimmune disease progression is at high risk of relapses or flares of the disease.

6. The method according to claim 4, wherein the autoimmune disease is type 1 diabetes, and
wherein an individual who is at low risk of autoimmune disease progression is at low risk of progressing to type 1 diabetes, and
wherein an individual who is at high risk of autoimmune disease progression is at high risk of progressing to type 1 diabetes.

7. The method according to claim 4, wherein the autoimmune disease is IPF, and
wherein an individual who is at low risk of autoimmune disease progression is at low risk of ongoing reduction of lung function, and
wherein an individual who is at high risk of autoimmune disease progression is at high risk of ongoing reduction of lung function.

8. The method according to claim 2, further comprising:
(ii) selecting an individual identified as one who is at high risk or low risk of autoimmune disease progression for treatment for the autoimmune disease.

9. The method according to claim 2, further comprising:
(ii) subjecting an individual identified as one who is at high risk or low risk of autoimmune disease progression to treatment for the autoimmune disease.

10. The method according to claim 8, wherein the treatment comprises inducing an exhausted CD8⁺ T cell or lack of CD4⁺ T cell costimulation phenotype in the individual.

11. The method according to claim 8, wherein an exhausted CD8⁺ T cell or lack of CD4⁺ T cell costimulation phenotype is induced in the individual by administering a therapeutically effective amount of a programmed cell death protein 1 (PD-1) ligand.

12. The method according to claim 11, wherein the PD-1 ligand is programmed death-ligand 1 (PDL-1).

13. The method according to claim 8, wherein the autoimmune disease is AAV or SLE and wherein individual has been identified as one who is at high risk of autoimmune disease progression,
wherein the treatment comprises treatment with a more frequent or more intense disease treatment regimen, or with a disease regimen not normally administered during the maintenance phase of the autoimmune disease.

14. The method according to claim 8, wherein the autoimmune disease is AAV or SLE and wherein individual has been identified as one who is at low risk of autoimmune disease progression,
wherein the treatment comprises treatment with a less frequent or less intense disease treatment regimen, or with a disease regimen not normally administered during the maintenance phase of the autoimmune disease.

15. The method according to claim 8, wherein individual has been identified as one who is at high risk of progressing to type 1 diabetes,
wherein the treatment comprises a prophylactic treatment for type 1 diabetes.

16. The method according to claim 8, wherein the autoimmune disease is IPF and wherein individual has been identified as one who is at high risk of autoimmune disease progression,
wherein the treatment comprises treatment with nintedanib, pirfenidone, a phosphodiesterase inhibitor, or immunosuppressive therapy.

17. An in vitro method of assessing whether CD8⁺ and CD4⁺ T cells in a sample have an exhausted CD8⁺ T cell or lack of CD4⁺ T cell costimulation phenotype, the method comprising establishing, by determining the expression level of two more genes selected from the group consisting of: KAT2B, CASK, ABCD2, DLG1, SS18, RBL2, RAB7L1, MTHFDI, KERA, BMI1, COG5, PDE4D, and VCY, whether said phenotype is present, wherein said phenotype is characterised by downregulated expression of genes KAT2B, CASK, ABCD2, DLG1, SS18, RBL2, RAB7L1, MTHFDI, BMI1, COG5, and PDE4D, and upregulated expression of genes KERA and VCY relative to the level of expression of these genes in a sample of CD8⁺ and CD4⁺ T cells which do not have said phenotype.

18. The in vitro method according to claim 17, wherein said expression level of the two or more genes is determined using reverse-transcription quantitative PCR (RT-qPCR), microarray analysis, enzyme-linked immunosorbent assays (ELISA), protein chips, flow cytometry (such as Flow-FISH for RNA, also referred to as FlowRNA), mass spectrometry, Western blotting, or northern blotting.

19. A method of assessing medical risk in an individual, wherein the assessment is of
(A)whether an individual with a chronic infection is at high risk or low risk of progression of said chronic infection; OR
(B)whether an individual is at high risk or low risk of not mounting an effective immune response to a vaccine against a disease, the individual having received the vaccination; OR
(C)whether an individual is at high risk or low risk of infection-associated immunopathology; OR
(D)whether an individual is at high risk or low risk of transplant rejection; OR
(E) assessing whether an individual is at high risk or low risk of cancer progression;
the method comprising either:
(i) determining whether the individual has or does not have an exhausted CD8⁺ T cell or lack of CD4⁺ T cell costimulation phenotype, said method being performed by using the method of claim 1; OR
determining whether the individual has or does not have an exhausted CD8⁺ T cell or lack of CD4⁺ T cell costimulation phenotype, by:
(ii) providing a PBMC sample obtained from the individual;
(iii) extracting mRNA from the PBMC sample;
(iv) performing reverse transcription quantitative PCR (RT-qPCR) to convert the mRNA into cDNA and determine the expression level of two more genes selected from the group consisting of: KAT2B, CASK, ABCD2, DLG1, SS18, RBL2, RAB7L1, MTHFDI, KERA, BMI1, COG5, PDE4D, and VCY, wherein said phenotype is characterised by down regulated expression of genes KAT2B, CASK, ABCD2, DLG1, SS18, RBL2, RAB7L1, MTHFDI, BMI1, COG5, and PDE4D, and upregulated expression of genes KERA and VCY, relative to the level of expression of these genes in an individual who does not have said phenotype, and
wherein the presence of said phenotype indicates in (A) that the individual is at high risk of progression of said chronic infection, and wherein the absence of said phenotype indicates in (A) that the individual is at low risk of progression of said chronic infection;
wherein the presence of said phenotype indicates in (B) that the individual at high risk of not mounting effective immune response to the vaccination, and wherein the absence of said phenotype indicates in (B) that the individual is at low risk of not mounting effective immune response to the vaccination;
wherein the presence of said phenotype indicates in (C) that the individual is at low risk of infection-associated immunopathology, and wherein the absence of said phenotype indicates in (C) that the individual is at high risk of infection-associated immunopathology;
wherein the presence of said phenotype indicates in (D) that the individual is at low risk of transplant rejection, and wherein the absence of said phenotype indicates in (D) that the individual is at high risk of transplant rejection;
wherein the presence of said phenotype indicates in (E) that the individual is at high risk of cancer progression, and wherein the absence of said phenotype indicates in (E) that the individual is at low risk of cancer progression.

20. The method as defined in claim 19, performed on a system comprising a tool or tools for determining expression of two more genes selected from the group consisting of: KAT2B, CASK, ABCD2, DLG1, SS18, RBL2, RAB7L1, MTHFDI, KERA, BMI1, COG5, PDE4D, and VCY;
and a computer programmed to compute a risk score of the risk of the individual not responding to the treatment from the gene expression data of the subject.
